SEQUENCE LISTING (18 sequences)

SEQ ID NO: 1 - protein, 398 aa, Homo sp.
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 2 - protein, 398 aa, synthetic construct
LDNGLARTPPMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNKTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAGPLFMSNDLRAISPQAKALLQDKDVIAINQDPLGKQGYQLRKGDNFEVWERPLSGDAWAVAIINRQEIGGPRGYTIPVASLGKGVACNPACFITQLLPVKRKLGFYEATSRLRSHINPTGTVLLQLENTMQTSLKDLL

SEQ ID NO: 3 - protein, 398 aa, synthetic construct
LDNGLARTPTMGWLHWERFMCNLDCQEEPDSCISEKLFMEMAERMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWASIKSILDWTSRNQERIVDVAGPGGWNDPDMLVIGNFGLSWDQQVTQMASWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRQLGFYNWTSRLKSHINPTGTVLLQLENTMQMSLKDLL

SEQ ID NO: 4 - protein, 31 aa, synthetic construct
MQLRNPELHLGCALALRFLALVSWDIPGARA

SEQ ID NO: 5 - protein, 429 aa, Homo sp.
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 6 - protein, 429 aa, synthetic construct
MQLRNPELHLGCALALRFLALVSWDIPGARALDNGLARTPPMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNKTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAGPLFMSNDLRAISPQAKALLQDKDVIAINQDPLGKQGYQLRKGDNFEVWERPLSGDAWAVAIINRQEIGGPRGYTIPVASLGKGVACNPACFITQLLPVKRKLGFYEATSRLRSHINPTGTVLLQLENTMQTSLKDLL

SEQ ID NO: 7 - protein, 429 aa, synthetic construct
MQLRNPELHLGCALALRFLALVSWDIPGARALDNGLARTPTMGWLHWERFMCNLDCQEEPDSCISEKLFMEMAERMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWASIKSILDWTSRNQERIVDVAGPGGWNDPDMLVIGNFGLSWDQQVTQMASWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRQLGFYNWTSRLKSHINPTGTVLLQLENTMQMSLKDLL

SEQ ID NO: 8 - DNA, 1290 bp, synthetic construct
  source 1..1290: mol_type = other DNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 9 - DNA, 1197 bp, unidentified
  source 1..1197: mol_type = other DNA
ttggacaatggactggcaagaactcctcccatgggctggctgcactgggaaaggtttatgtgtaatctggactgtcaagaggagccagattcctgcatctctgagaagctctttgaagagatggctgagaggatggtgacagatggatggaaggatgctggatatgagtacctgtgcattgatgactgctggatggcccctcagagagacagtgaaggcagactgcaagcagacccccagaggttcccccatggcattaggcagctggccaaccatgtccactccaagggcctgaaactgggcatctatgctgatgtgggcaataagacctgtgctggtttccctggctcctttggatactatgacattgatgcccagacctttgctgactggggtgtggacctgctcaagtttgatggctgctactgtgactccctggagaacctggctgatggttacaagcacatgtctcttgctctgaacaagactggtagacccattgtttacagctgtgagtggccactgtacatgtggcccttccagaagcccaactacactgagatcagacagtactgcaatcattggaggaattttgcagatattgatgattcttgggcctccatcaagagcatcctggactggacttccagaaaccaagaaagaattgtggatgttgctggccctggaggttggaatgaccctgacatgctggtgattggcaactttgggctgtcctgggaccagcaagtgactcagatggccctctgggccatcatggctgggcccctcttcatgtccaatgatctgagggccatttccccccaagccaaggccctgcttcaagacaaagatgtcattgctatcaatcaagatcccctgggcaagcaaggctaccagctcagaaaaggagacaactttgaggtgtgggagagacctctgtctggagatgcctgggctgtggccatcatcaacagacaagagattggtggccccagaggctacaccatccctgtggcttccctggggaagggtgttgcctgcaacccagcttgcttcatcacccagcttctgccagtgaagaggaagctgggcttctatgaagctaccagcagactgagatcccacatcaaccccactggcacagtgctgctgcagctggaaaacaccatgcagacttctctgaaggacctcctgtga

SEQ ID NO: 10 - DNA, 1197 bp, unidentified
  source 1..1197: mol_type = other DNA
ctggataatggactggcaagaaccccaaccatgggttggctgcactgggagaggtttatgtgtaatctggactgtcaagaggagccagactcctgcatctctgagaagctcttcatggagatggctgagaggatggtgagtgaaggctggaaggatgctggttatgagtacctgtgcattgatgactgctggatggcccctcagagagattcagagggcagactgcaagcagatccccagaggttcccccatggcattaggcaactggccaactatgtccactccaagggcctgaagctgggcatctatgctgatgtgggcaacaagacctgtgctggcttccctggctcctttggctactatgatattgatgcccagacctttgctgactggggtgtggacctgctcaagtttgatggctgctactgtgacagcctggagaacctggctgatggttacaagcacatgtctcttgctctgaacagaactggtagaagcattgtttacagctgtgagtggccactgtacatgtggcccttccagaagcccaactacactgagatcagacagtactgcaaccattggaggaactttgcagacattgatgattcctgggcctccatcaagagcatcctggactggacttccagaaaccaagagagaattgtggatgttgctggacctggaggatggaatgaccctgacatgctggtgattggaaattttgggctgtcctgggaccagcaagtgactcagatggccagctgggccatcatggcagcccccctgttcatgagcaatgatctcagacacatttccccccaagccaaggccctgcttcaagacaaagatgtcattgctattaatcaagatcctttgggcaagcaaggctaccagctgaggaagggagacaactttgaggtgtgggaaagacctctgtctggagatgcctgggctgtggctatcatcaatagacaagaaattggtggccccagatcctacaccatccctgttgcctctctgggaaaaggagtggcctgcaatccagcttgcttcatcacccagctcctcccagtgaagaggcagctgggtttctacaactggacaagcagattgaagtcccacatcaaccccactggcacagtgctgctgcagctggaaaacaccatgcagatgtccctgaaggacctcctgtga

SEQ ID NO: 11 - DNA, 93 bp, unidentified
  source 1..93: mol_type = other DNA
atgcagctgaggaacccagaactacatctgggctgcgcgcttgcgcttcgcttcctggccctcgtttcctgggacatccctggggctagagca

SEQ ID NO: 12 - DNA, 1290 bp, unidentified
  source 1..1290: mol_type = other DNA
atgcagctgaggaacccagaactacatctgggctgcgcgcttgcgcttcgcttcctggccctcgtttcctgggacatccctggggctagagcattggacaatggactggcaagaactcctcccatgggctggctgcactgggaaaggtttatgtgtaatctggactgtcaagaggagccagattcctgcatctctgagaagctctttgaagagatggctgagaggatggtgacagatggatggaaggatgctggatatgagtacctgtgcattgatgactgctggatggcccctcagagagacagtgaaggcagactgcaagcagacccccagaggttcccccatggcattaggcagctggccaaccatgtccactccaagggcctgaaactgggcatctatgctgatgtgggcaataagacctgtgctggtttccctggctcctttggatactatgacattgatgcccagacctttgctgactggggtgtggacctgctcaagtttgatggctgctactgtgactccctggagaacctggctgatggttacaagcacatgtctcttgctctgaacaagactggtagacccattgtttacagctgtgagtggccactgtacatgtggcccttccagaagcccaactacactgagatcagacagtactgcaatcattggaggaattttgcagatattgatgattcttgggcctccatcaagagcatcctggactggacttccagaaaccaagaaagaattgtggatgttgctggccctggaggttggaatgaccctgacatgctggtgattggcaactttgggctgtcctgggaccagcaagtgactcagatggccctctgggccatcatggctgggcccctcttcatgtccaatgatctgagggccatttccccccaagccaaggccctgcttcaagacaaagatgtcattgctatcaatcaagatcccctgggcaagcaaggctaccagctcagaaaaggagacaactttgaggtgtgggagagacctctgtctggagatgcctgggctgtggccatcatcaacagacaagagattggtggccccagaggctacaccatccctgtggcttccctggggaagggtgttgcctgcaacccagcttgcttcatcacccagcttctgccagtgaagaggaagctgggcttctatgaagctaccagcagactgagatcccacatcaaccccactggcacagtgctgctgcagctggaaaacaccatgcagacttctctgaaggacctcctgtga

SEQ ID NO: 13 - DNA, 1290 bp, unidentified
  source 1..1290: mol_type = other DNA
atgcagctgaggaacccagaactacatctgggctgcgcgcttgcgcttcgcttcctggccctcgtttcctgggacatccctggggctagagcactggataatggactggcaagaaccccaaccatgggttggctgcactgggagaggtttatgtgtaatctggactgtcaagaggagccagactcctgcatctctgagaagctcttcatggagatggctgagaggatggtgagtgaaggctggaaggatgctggttatgagtacctgtgcattgatgactgctggatggcccctcagagagattcagagggcagactgcaagcagatccccagaggttcccccatggcattaggcaactggccaactatgtccactccaagggcctgaagctgggcatctatgctgatgtgggcaacaagacctgtgctggcttccctggctcctttggctactatgatattgatgcccagacctttgctgactggggtgtggacctgctcaagtttgatggctgctactgtgacagcctggagaacctggctgatggttacaagcacatgtctcttgctctgaacagaactggtagaagcattgtttacagctgtgagtggccactgtacatgtggcccttccagaagcccaactacactgagatcagacagtactgcaaccattggaggaactttgcagacattgatgattcctgggcctccatcaagagcatcctggactggacttccagaaaccaagagagaattgtggatgttgctggacctggaggatggaatgaccctgacatgctggtgattggaaattttgggctgtcctgggaccagcaagtgactcagatggccagctgggccatcatggcagcccccctgttcatgagcaatgatctcagacacatttccccccaagccaaggccctgcttcaagacaaagatgtcattgctattaatcaagatcctttgggcaagcaaggctaccagctgaggaagggagacaactttgaggtgtgggaaagacctctgtctggagatgcctgggctgtggctatcatcaatagacaagaaattggtggccccagatcctacaccatccctgttgcctctctgggaaaaggagtggcctgcaatccagcttgcttcatcacccagctcctcccagtgaagaggcagctgggtttctacaactggacaagcagattgaagtcccacatcaaccccactggcacagtgctgctgcagctggaaaacaccatgcagatgtccctgaaggacctcctgtga

SEQ ID NO: 14 - DNA, 130 bp, unidentified
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 15 - DNA, 1881 bp, unidentified
  source 1..1881: mol_type = other DNA
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatca

SEQ ID NO: 16 - DNA, 592 bp, synthetic construct
  source 1..592: mol_type = other DNA
aatcaacctctggattacaaaatttgtgaaagattgactggtattcttaactttgttgctccttttacgctttgtggatacgctgctttattgcctttgtatcttgctattgcttcccgtttggctttcattttctcctccttgtataaatcctggttgctgtctctttttgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatc

SEQ ID NO: 17 - DNA, 234 bp, unidentified
  source 1..234: mol_type = other DNA
cctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaa

SEQ ID NO: 18 - DNA, 133 bp, synthetic construct
  source 1..133: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagaga

(19) United States
(12) Patent Application Publication
(10) Pub. No.: US 2025/0177572 A1
(43) Pub. Date: Jun. 5, 2025

(54) COMPOSITION FOR USE IN THE TREATMENT OF FABRY DISEASE

(71) Applicant: TAKEDA PHARMACEUTICAL COMPANY LIMITED, Osaka (JP)

(72) Inventors: Rizwana Islam, Cambridge, MA (US); Wanida Ruangsiriluk, Cambridge, MA (US); Mugdha Ravindra Deshpande, Cambridge, MA (US)

(21) Appl. No.: 19/060,259

(22) Filed: Feb. 21, 2025

Related U.S. Application Data
(63) Continuation of application No. PCT/IB2023/058411, filed on Aug. 24, 2023.
(60) Provisional application No. 63/500,742, filed on May 8, 2023.
(60) Provisional application No. 63/482,948, filed on Feb. 2, 2023.
(60) Provisional application No. 63/401,089, filed on Aug. 25, 2022.

(51) Int. Cl. A61K 48/00; A61K 9/00; A61P 25/02

(52) U.S. Cl. CPC A61K 48/0058; A61K 9/0019; A61K 48/0008; A61P 25/02

(57) ABSTRACT

The present disclosure provides, among other things, gene therapy approaches of treating Fabry disease in a subject, particularly for alleviating periphery neuropathy associated with Fabry disease. The method comprises administering to a subject in need thereof a recombinant adeno-associated viral vector (rAAV) packaged in an AAV capsid having broad tissue tropism, and expressing a GLA transgene with improved stability and lowered immunogenicity.

### CROSS REFERENCED APPLICATIONS

This application is a Continuation Application of International Application No. PCT/IB2023/058411, filed on Aug. 24, 2023, which claims priority to, and the benefit of U.S. Provisional Patent Application Ser. No. 63/401,089 filed on Aug. 25, 2022; U.S. Provisional Patent Application Ser. No. 63/482,948, filed on Feb. 2, 2023; and U.S. Provisional Patent Application Ser. No. 63/500,742 filed on May 8, 2023; the contents of each of which are incorporated herein by reference in their entireties.

### INCORPORATION BY REFERENCE OF SEQUENCE LISTING

The instant application contains a sequence listing which has been submitted electronically in XML format and is hereby incorporated by reference in its entirety. The sequence listing file entitled MIL-022WO1.XML, was created on Aug. 21, 2023, which is 28,159 bytes in size.

### BACKGROUND

Fabry disease is rare a progressive congenital metabolic disease caused by a deficiency in the lysosomal enzyme α-galactosidase A (α-GAL) as a result of mutations in the GLA gene. Fabry diseases affects 1 in 40,000 males, who develop multisystemic disease that typically develops in childhood or adolescence. Fabry disease can also affect females and manifest with a wide range of symptoms. Lack of α-GAL enzyme activity results in the progressive, systematic accumulation of its primary substrate, globotriaosylceramide (GB3) and its deacetylated soluble form, globotriaosylsphingosine (lysoGb3). If left untreated, Fabry patients have a reduced life expectancy, often dying around the age of forty or fifty due to vascular disease affecting the kidneys, heart and/or central nervous system.

The neurological manifestations of Fabry disease include the peripheral nervous system, with globotriaosylceramide accumulation found in Schwann cells and dorsal root ganglia. Fabry disease in some patients is associated with the development of pain, possibly caused by the deposition of lipids in the dorsal root ganglia and sympathetic ganglia, or by small fiber neuropathy. Peripheral neuropathy affects over a quarter of patients with Fabry disease and is characterized by loss of small myelinated and non-myelinated fibers, whereas larger fibers are largely unaffected (Ohnishi and Dyck. Loss of small peripheral sensory neurons in Fabry disease. Arch Neurol 1974; 31:120)

Enzyme replacement therapy (“ERT”) by introducing functional enzyme is currently the approved treatment of Fabry disease. While ERT is effective in many cases, this treatment requires life-long intravenous administration of α-GAL every two weeks. ERT resolves symptoms associated with Fabry disease but is not curative and does not stop disease progression. The insufficient pharmacologic response is largely due to the short circulatory half-life of the enzyme and suboptimal cellular delivery. Thus, there remains a need for therapies for treating Fabry disease that can stop disease progression and potentially be curative. Gene therapy is a promising treatment.

### SUMMARY OF THE INVENTION

The present application relates to gene therapy approaches using recombinant adeno associated viral vectors (rAAV) to mediate transfer and expression of the α-Galactosidase A (GLA) gene. In particular, the application discloses gene therapy methods and compositions, among other things for alleviating, treating and/or preventing peripheral neuropathy in Fabry disease. The rAAV vector expressing a GLA transgene leads to sustained exposure of functional α-GAL enzyme in multiple tissues at high levels in Fabry patients, comparable to or higher than normal enzymatic function in healthy persons, particularly in the nervous system (e.g., the PNS). The restored α-GAL reduces the accumulation of substrates Gb3 and lysoGb3 in the nervous tissues, particularly in the dorsal root ganglion, and ameliorates nerve abnormalities including preserving the density of small nerve fibers (e.g., myelinated nerve fibers).

In some embodiments, the rAAV vector used to treat and/or prevent peripheral neuropathy in Fabry patients according to the present disclosure has broad tissue tropism and utilizes a ubiquitous promoter to drive widespread gene expression, resulting in sustained high levels of protein expression and robust protein exposure to a wide range of tissues and/or decrease in Gb3 or lysoGb3 levels. In addition, the GLA transgene used in the present application is codon optimized and/or expresses an engineered variant of α-GAL, resulting in an increase in α-GAL activity in vivo, and/or a decrease in lysoGb3 or Gb3 levels in vivo. Additionally, the present gene approach to drive expression of GLA variants that encode α-GAL protein with increased half-life and improved cellular uptake provides further increases in α-GAL exposure in key target tissues, e.g., the nervous tissues, allowing better treatment outcomes of Fabry disease, for example, for improved peripheral neuropathy outcomes.

In some embodiments described herein, a liver or a nervous system specific promoter may be used.

As described in more detail below, the rAAV based gene therapy approach described herein results in overall improvement in health as evidenced by gain in body mass, improved kidney function, and neurological symptoms in Fabry disease mouse models and is further expected to elicit the same in humans.

In particular, the present gene therapy approach is suitable for alleviating peripheral neuropathy in Fabry disease. Accordingly, in one aspect of the present invention, a method for treating, alleviating and/or preventing peripheral neuropathy in a subject diagnosed with Fabry disease is provided; the method comprises administering to the subject a composition comprising a recombinant adeno-associated viral vector (rAAV) that comprises a polynucleotide encoding α-GAL enzyme or a variant thereof. The symptoms of peripheral neuropathy may manifest as neuropathic pain, thermal hypoesthesia, hearing loss, other sensory deficiencies and/or gastrointestinal disturbances.

In some embodiments, a method for improving peripheral abnormalities in nerve fibers in a subject with Fabry disease is provided; the method comprises administering to the subject a composition comprising a recombinant adeno-associated viral vector (rAAV) that comprises a polynucleotide encoding α-GAL enzyme or a variant thereof. In some embodiments, the small nerve fibers are preserved, including small myelinated and unmyelinated nerve fibers. In some embodiments, the density of small sensory nerves is preserved.

In some embodiments, the rAAV vector expressing a GLA transgene as described herein is used to reduce the accumulation of globotriaosylceramide (Gb3) in the peripheral nervous system caused by α-galactosidase A deficiency comprising administering to a subject in need. In some embodiments, the accumulation of Gb3 in the dorsal root ganglion (DRG) is reduced. In other embodiments, expression of LAMP1 is decreased in the DRG.

In some embodiments, the rAAV vector expressing the GLA transgene has broad tissue tropism, said vector comprising: (a) a 5′ inverted terminal repeat (ITR); (b) a ubiquitous promoter; (c) a polynucleotide encoding α-GAL enzyme or variant thereof; (d) a poly A; and (e) a 3′ ITR.

In some embodiments, the rAAV vector that may be used to treat and/or prevent peripheral neuropathy in Fabry patients exhibits broad tissue tropism and comprises: (a) a 5′ inverted terminal repeat (ITR); (b) a ubiquitous promoter; (c) a polynucleotide encoding α-GAL enzyme or variant thereof; (d) a woodchuck hepatitis virus posttranscriptional regulatory element (WPRE); (e) a poly A; and (f) a 3′ ITR.

In some embodiments, the rAAV vector that may be used to treat and/or prevent peripheral neuropathy in Fabry patients comprises: (a) a 5′ inverted terminal repeat (ITR); (b) a liver specific promoter; (c) a polynucleotide encoding α-GAL enzyme or variant thereof; (d) a poly A; and (e) a 3′ ITR. In other embodiments, the rAAV vector further comprises a woodchuck hepatitis virus posttranscriptional regulatory element (WPRE). The liver specific promotor induces expression of α-GAL enzyme in the liver which can cross the brain-blood barrier (BBB) to the nervous system, e.g., to the PNS.

In some embodiments, the rAAV vector as described herein is packaged with an AAV capsid with broad tissue tropism.

Various kinds of AAV capsids with broad tissue tropism can be used in the rAAV vector described herein. For example, in some embodiments, the AAV capsid is a wide-tropism AAV capsid selected from an AAV1 capsid, AAV2 capsid, AAV3 capsid, AAV4 capsid, AAV5 capsid, AAV6 capsid, AAV7 capsid, AAV8 capsid, AAV9 capsid, AAV11, 12,13, AAVhu.37, AAVrh.8, AAVrh.10, and AAVrh.39, AAV-DJ, or AAV-DJ/8.

Accordingly, in some embodiments, the AAV capsid with wide-tropism comprises an AAV1 capsid. In some embodiments, the AAV capsid with wide-tropism is comprises an AAV2 capsid. In some embodiments, the AAV capsid with wide-tropism comprises an AAV3 capsid. In some embodiments, the AAV capsid with wide-tropism comprises an AAV4 capsid. In some embodiments, the AAV capsid with wide-tropism comprises an AAV5 capsid. In some embodiments, the AAV capsid with wide-tropism comprises an AAV6 capsid. In some embodiments, the AAV capsid with wide-tropism comprises an AAV7 capsid. In some embodiments, the AAV capsid with wide-tropism comprises an AAV8 capsid. In some embodiments, the AAV capsid with wide-tropism comprises an AAV9 capsid.

In one embodiment, the AAV capsid comprise an AAV9 capsid. The AAV9 capsid is naturally occurring or modified.

In some embodiments, the ubiquitous promoter is selected from chicken β actin (CBA) promoter, CAG promoter, EF-1α promoter, PGK promoter, UBC promoter, LSE beta-glucuronidase (GUSB) promoter, or ubiquitous chromatin opening element (UCOE) promoter. In some embodiments, the ubiquitous promoter comprises CBh (CMV enhancer, Chicken beta-actin promoter, Chicken-beta actin-MVM hybrid intron). Accordingly, in some embodiments, the ubiquitous promoter is a chicken β actin (CBA) promoter. In some embodiments, the ubiquitous promoter is an EF-1α promoter. In some embodiments, the EF-1α promoter is in combination with chimeric intron from chicken β-actin and rabbit β-globin genes. In some embodiments, the ubiquitous promoter is a UBC promoter. In some embodiments, the ubiquitous promoter is an LSE beta-glucuronidase (GUSB) promoter. In some embodiments, the ubiquitous promoter is a ubiquitous chromatin opening element (UCOE) promoter.

In some embodiments, the ubiquitous promoter comprises a cyto-megalo-virus (CMV) enhancer, chicken beta actin promoter, and a rabbit beta globin intron.

In some embodiments, the ubiquitous promoter comprises a shortened EF-1α promoter and one or more introns.

In some embodiments, the one or more introns are from chicken β-actin and/or rabbit β-globin genes.

In some embodiments, the rAAV vector with broad tissue tropism comprises a liver specific promoter. Exemplary liver-specific promoters include, but are not limited to, for example, transthyretin promoter (TTR); thyroxine binding globulin (TBG) promoter; hybrid liver-specific promoter (HLP), and alpha-1-antitrypsin (AAT) promoter.

In some embodiments, the WPRE sequence is optional or is modified. In one embodiment, the WPRE sequence is WPRE mut6delATG.

Exemplary polyA sequences that may be included in the gene therapy vectors encompassed by the present disclosure include human growth hormone polyA (hGHpA), synthetic polyA (SPA), Simian virus 40 late poly A (SV 40pA) and bovine growth hormone (BGH) poly A. In a particular embodiment, the poly A is bovine growth hormone (BGH) poly A.

In some embodiments, the GLA transgene expressing α-GAL enzyme comprises a GLA gene having a wild type sequence (SEQ ID Nos: 1 and 5) or a GLA gene having a modified sequence described herein. Such modified GLA sequences include, for example, codon optimized GLA and/or engineered variants of GLA.

In some embodiments, the nucleotide sequence encoding α-GAL enzyme is codon optimized. In some examples, the nucleotide sequence encoding α-GAL enzyme is codon optimized for human cells.

In some embodiments, the α-GAL enzyme has an unmodified sequence. In other embodiments, the α-GAL enzyme has a modified sequence.

In some embodiments, the nucleotide sequence encoding the α-GAL enzyme is engineered. In some embodiments, the nucleotide sequence encoding the α-GAL enzyme is engineered and codon optimized. In some embodiments, the modified sequence comprises one or more amino acid substitutions in comparison to wild-type α-GAL enzyme (SEQ ID NO: 5). In some embodiments, the modified α-GAL enzyme variant has increased stability (e.g., serum stability), intracellular activity (e.g., lysosomal activity), and/or specific catalytic activity, in comparison to wild-type α-GAL enzyme.

As non-limiting examples, the α-GAL enzyme variant comprises the amino acid sequence of SEQ ID NO: 2. In some embodiments, the α-GAL enzyme variant comprises the amino acid sequence of SEQ ID NO: 3. In some embodiments, the α-GAL enzyme variant comprises the amino acid sequence of SEQ ID NO: 6. In some embodiments, the α-GAL enzyme variant comprises the amino acid sequence of SEQ ID NO: 7.

As non-limiting examples, the GLA transgene comprises a nucleotide sequence selected from SEQ ID Nos: 8-10 and 12-13. In some embodiments, the GLA transgene comprises the nucleotide sequence of SEQ ID NO: 8. In some embodiments, the GLA transgene comprises the nucleotide sequence of SEQ ID NO: 9. In some embodiments, the GLA transgene comprises the nucleotide sequence of SEQ ID NO: 10. In some embodiments, the GLA transgene comprises the nucleotide sequence of SEQ ID NO: 12. In some embodiments, the GLA transgene comprises the nucleotide sequence of SEQ ID NO: 13.

As non-limiting examples, the present method comprises administering to a subject in need thereof a rAAV vector packaged in a rAAV9 capsid having broad tissue tropism, the vector comprising: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding α-GAL enzyme or variant thereof; (d) a poly A; and (e) a 3′ ITR.

In another example, the present method comprises administering to a Fabry patient exhibiting symptoms of peripheral neuropathy, a rAAV vector packaged in a rAAV9 capsid having broad tissue tropism, the vector comprising: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding α-GAL enzyme; (d) a woodchuck hepatitis virus posttranscriptional regulatory element (WPRE); (e) a poly A; and (f) a 3′ ITR.

In some embodiments, the rAAV vector is administered by intravenous, subcutaneous, or transdermal administration. Accordingly, in some embodiments, the rAAV vector is administered intravenously to a subject in need thereof. In some embodiments, the rAAV vector is administered subcutaneously to a subject in need thereof. In some embodiments, the rAAV vector is administered transdermally to a subject in need thereof.

In some embodiments, the transdermal administration is by gene gun.

In some embodiments, the rAAV vector of the present disclosure is administered at a dose ranging from 1.0×10¹⁰ vg/kg (viral genome/kilogram of body weight) to 1.0×10¹⁴ vg/kg. In some embodiments, the rAAV vector of the present disclosure is administered at a dose ranging from 1.0×10¹⁰ vg/kg to 5.0×10¹³ vg/kg. In some embodiments, the rAAV vector of the present disclosure is administered at a dose ranging from 1.0×10¹⁰ vg/kg to 1.0×10¹³ vg/kg. In some embodiments, the rAAV vector of the present disclosure is administered at a dose ranging from 1.0×10¹⁰ vg/kg to 5.0×10¹² vg/kg. In some embodiments, the rAAV vector of the present disclosure is administered at a dose ranging from 1.0×10¹⁰ vg/kg to 1.0×10¹² vg/kg. In some embodiments, the rAAV vector of the present disclosure is administered at a dose ranging from 1.0×10¹⁰ vg/kg to 5.0×10¹¹ vg/kg. In some embodiments, the rAAV vector of the present disclosure is administered at a dose ranging from 1.0×10¹⁰ vg/kg to 2.5×10¹¹ vg/kg. In some embodiments, the rAAV vector of the present disclosure is administered at a dose ranging from 5.0×10¹⁰ vg/kg to 1.0×10¹⁴ vg/kg. In some embodiments, the rAAV vector of the present disclosure is administered at a dose ranging from 5.0×10¹⁰ vg/kg to 5.0×10¹³ vg/kg. In some embodiments, the rAAV vector of the present disclosure is administered at a dose ranging from 5.0×10¹⁰ vg/kg to 1.0×10¹³ vg/kg. In some embodiments, the rAAV vector of the present disclosure is administered at a dose ranging from 5.0×10¹⁰ vg/kg to 5.0×10¹² vg/kg. In some embodiments, the rAAV vector of the present disclosure is administered at a dose ranging from 5.0×10¹⁰ vg/kg to 1.0×10¹² vg/kg. In some embodiments, the rAAV vector of the present disclosure is administered at a dose ranging from 5.0×10¹⁰ vg/kg to 5.0×10¹¹ vg/kg. In some embodiments, the rAAV vector of the present disclosure is administered at a dose ranging from 5.0×10¹⁰ vg/kg to 2.5×10¹¹ vg/kg.

In some embodiments, following administration of the rAAV vector, the subject has detectable α-GAL in the serum for at least 5 weeks, 10 weeks, 18 weeks, 15 weeks, 26 weeks, 1 year, 5 years, 10 years, or 20 years. In some embodiments, following administration of the rAAV vector, the subject has detectable α-GAL in the serum for at least 5 weeks. In some embodiments, following administration of the rAAV vector, the subject has detectable α-GAL in the serum for at least 10 weeks. In some embodiments, following administration of the rAAV vector, the subject has detectable α-GAL in the serum for at least 15 weeks. In some embodiments, following administration of the rAAV vector, the subject has detectable α-GAL in the serum for at least 26 weeks. In some embodiments, following administration of the rAAV vector, the subject has detectable α-GAL in the serum for at least 1 year. In some embodiments, following administration of the rAAV vector, the subject has detectable α-GAL in the serum for at least 5 years. In some embodiments, following administration of the rAAV vector, the subject has detectable α-GAL in the serum for at least 10 years. In some embodiments, following administration of the rAAV vector, the subject has detectable α-GAL in the serum for at least 15 years. In some embodiments, following administration of the rAAV vector, the subject has detectable α-GAL in the serum for at least 20 years. In some embodiments, following administration of the rAAV vector, the subject has detectable α-GAL in the serum for the life of the subject.

In some embodiments, expression of modified α-GAL enzyme provides 3, 10, 30, 100, 300, 1000, 3000, 10,000, 15,000, 20,000, 25,000, 30,000 folds higher serum α-GAL levels compared to the expression of WT α-GAL. In some embodiments, expression of modified α-GAL enzyme provides, 3, 10, 30, 100, 1000, 3000, 10,000, 15,000, 20,000, 30,000 folds higher intracellular enzyme levels compared to expression of WT α-GAL.

In some embodiments, the administration results in α-GAL enzyme exposure in one or more of liver, kidney, heart, gastrointestinal tract, brain, and/or peripheral neurons of the subject. Accordingly, in some embodiments, administration results in α-GAL enzyme exposure in the liver. In some embodiments, administration results in α-GAL enzyme exposure in the kidney. In some embodiments, administration results in α-GAL enzyme exposure in the heart. In some embodiments, administration results in α-GAL enzyme exposure in the gastrointestinal tract and cells associated with the gastrointestinal tract. In some embodiments, administration results in α-GAL enzyme exposure in the brain. In some embodiments, administration results in α-GAL enzyme exposure in peripheral neurons.

In another aspect, the present invention provides a method for alleviating or ameliorating a gastrointestinal symptom in a subject diagnosed with Fabry disease comprising administering to the subject a composition comprising a recombinant adeno-associated viral vector (rAAV) that comprises a polynucleotide encoding α-GAL enzyme or a variant thereof, wherein the subject diagnosed with Fabry disease has or is developing one or more gastrointestinal symptoms. The gastrointestinal symptoms include intestinal dysmotility, impaired autonomic function, vasculopathy and myopathy. In some embodiments, the treatment reverses vacuolization in the gastrointestinal tract.

### BRIEF DESCRIPTION OF THE DRAWINGS

FIG. 1 illustrates an exemplary vector diagram of rAAV9 comprising a wild type α-GAL under the control of a ubiquitous promoter (referred to generally herein as “rAAV9”) as described herein.

FIG. 2A is a histogram of α-Gal activity in serum 18 weeks after administration of rAAV vectors expressing the GLA transgenes at two different doses: 5×10¹⁰ and 2.5×10¹¹ vg/kg, and controls as shown in Table 4.

FIGS. 2B-2D show α-Gal activities in different tissues: kidney (2B), heart (2C) and liver (2D) 18 weeks after administration of rAAV vectors expressing the GLA transgenes at two different doses: 5×10¹⁰ and 2.5×10¹¹ vg/kg, and controls as shown in Table 4. FIG. 2E is a graph showing alpha galactosidase activity in kidney and heart of Fabry mice treated with variant 1 or wild-type human alpha galactosidase. FIG. 2F is a graph showing alpha galactosidase activity in vehicle control mice, wild-type alpha galactosidase transduced Fabry mice and variant 1 transduced Fabry mice. FIG. 2G is a graph showing serum alpha galactosidase concentration in primate transduced with AAV-variant 1 at different concentrations −6.25e12 or 3e13 vg/kg.

FIGS. 3A-3E show Gb3 substrate concentrations in serum (3A), kidney (3B), heart (3C) and liver (3D) 18 weeks after administration of rAAV vectors expressing the GLA transgenes at two different doses: 5×10¹⁰ and 2.5×10¹¹ vg/kg, and controls as shown in Table 4. FIG. 3E is a graph showing percent reduction in Gb3 volume in kidney or heart when treated with variant 1 or vehicle control.

FIGS. 4A-4D show lysoGb3 substrate concentrations in in serum (4A), kidney (4B), heart (4C) and liver (4D) 18 weeks after administration of rAAV vectors expressing the GLA transgenes at two different doses: 5×10¹⁰ and 2.5×10¹¹ vg/kg, and controls as shown in Table 4.

FIG. 5 illustrates hot plate latency of mice 18 weeks after administering rAAV vectors expressing the GLA transgenes at two different doses: 5×10¹⁰ and 2.5×10¹¹ vg/kg, and controls as shown in Table 4.

FIG. 6 shows representatives of MPZ IHC staining of DRGs in WT mice, Gb3Stg/GLAko mice treated with rAAV9-Variant 1 at a dose of 2.5×10¹¹ vg/kg and Gb3Stg/GLAko mice administered with a rAAV9 null vector.

FIG. 7A shows immunohistochemistry staining of the kidney tissues. The results show supraphysiological α-Gal A enzyme exposure in target cells (podocytes) in the kidney in Gb3Stg/GLAko mice treated with the rAAV9-α-Gal gene therapy vector at a dose of 2.5×10¹¹ vg/kg. A rAAV9-Null vector is used as control.

FIG. 7B shows immunohistochemistry staining of the heart tissues. The results show supraphysiological α-Gal A enzyme exposure in target cells (cardiomyocytes) in the hearts in Gb3Stg/GLAko mice treated with the rAAV9-α-Gal gene therapy vector at a dose of 2.5×10¹¹ vg/kg. A rAAV9-Null vector is used as control.

FIG. 8A shows normalization of vacuolization and lysosomal burden in the dorsal nerve root (DNR) in Gb3Stg/GLAko mice treated with the rAAV9-α-Gal gene therapy construct at a dose of 2.5×10¹¹ vg/kg. A rAAV9-Null vector is used as control. The upper panel is the HE (hematoxylin and eosin) staining of the dorsal nerve root. The lower panel is the immunohistochemistry staining of LAMP1, lysosomal-associated membrane protein 1. FIG. 8B is a graph showing the percentage of LAMP1 positive area in Fabry mice, at baseline, treated with control or variant 1 gene therapy and normal mice. FIG. 8C is a graph showing hot plate latencies in Fabry mice treated with control or variant 1 gene therapy and normal mice. FIG. 8D is a graph showing correlation between DRG and latency. FIG. 8E is an MRI image showing DRG volume in Normal mice and Fabry mice transduced with vehicle control or gene therapy (GT), expressing a WT aGAL. FIG. 8F is a graph showing DRG volume in Fabry mice treated with vehicle control or gene therapy expressing WT aGAL or normal mice.

FIG. 9 shows reversal of pathological vacuolization and lysosomal burden in the gastrointestinal tract in in Gb3Stg/GLAko mice treated with the rAAV9-α-Gal gene therapy vector at a dose of 2.5×10¹¹ vg/kg. A rAAV9-Null vector is used as control. The upper panel is the HE (hematoxylin and eosin) staining of the transverse smooth muscles. Vacuoles in the transverse smooth muscles of G3Stg/GlaKO duodenum are reversed after treating with the rAAV9-α-Gal gene therapy vector. The middle panel shows the α-Gal A enzyme exposure. α-Gal A-positive staining is observed in mice treated with the rAAV9-α-Gal gene therapy vector. The lower panel is the immunohistochemistry staining of LAMP1, lysosomal-associated membrane protein 1. LAMP1-positive staining in smooth muscles and myenteric ganglion cells is observed only in untreated and rAAV9-Null-vector treated mice.

FIG. 10A shows IHC of hepatocytes treated with rAAV8-hα-GAL with a ubiquitous promoter. FIG. 10B shows IHC of hepatocytes treated with rAAV9-hα-GAL with a liver specific promoter.

FIG. 11A shows projected pharmacokinetics in terms of alpha galactosidase concentration in heart, kidney, liver and plasma at 3e11 vg/kg in human beings. FIG. 11B shows projected pharmacokinetics in terms of alpha galactosidase concentration in heart, kidney, liver and plasma at 1e11 vg/kg in human beings. FIG. 11C shows projected pharmacokinetics in terms of alpha galactosidase concentration in heart, kidney, liver and plasma if treated in the future with ERT in human beings. FIG. 11D shows projected pharmacokinetics in terms of alpha galactosidase concentration in heart, kidney, liver and plasma if treated in the future at 3e11 vg/kg in human beings. FIG. 11E shows projected pharmacokinetics in terms of Gb3 reduction in heart, kidney, liver and plasma at if treated in the future with 1e11 vg/kg in human beings. FIG. 11F shows projected pharmacokinetics of in terms of Gb3 reduction in heart, kidney, liver and plasma if treated in the future treated with ERT in human beings.

FIG. 12A is a graph showing alpha galactosidase activity over 4 weeks in mice when injected with rAAV9-hα-GAL variant 1 and rAAV9-hα-GAL-variant 2 at 2 different dosages. FIG. 12B is a bar graph showing alpha galactosidase activity in terminal serum at 2 dosages of rAAV9-hα-GAL variant 1 and rAAV9-hα-GAL-variant 2, as compared to controls. FIG. 12C is a bar graph showing alpha galactosidase activity in kidney at 2 dosages of rAAV9-hα-GAL variant 1 and rAAV9-hα-GAL-variant 2, as compared to controls. FIG. 12D is a bar graph showing alpha galactosidase activity in heart at 2 dosages of rAAV9-hα-GAL variant 1 and rAAV9-hα-GAL-variant 2, as compared to controls. FIG. 12E is a bar graph showing alpha galactosidase activity in liver at 2 dosages of rAAV9-hα-GAL variant 1 and rAAV9-hα-GAL-variant 2, as compared to controls. FIG. 12F is a bar graph showing Gb3 substrate levels in terminal serum at 2 dosages of rAAV9-hα-GAL variant 1 and rAAV9-hα-GAL-variant 2, as compared to controls. FIG. 12G is a bar graph showing Gb3 substrate levels in kidney at 2 dosages of rAAV9-hα-GAL variant 1 and rAAV9-hα-GAL-variant 2, as compared to controls. FIG. 12H is a bar graph showing Gb3 substrate levels in kidney at 2 dosages of rAAV9-hα-GAL variant 1 and rAAV9-hα-GAL-variant 2, as compared to controls. FIG. 12I is a bar graph showing Gb3 substrate levels in liver at 2 dosages of rAAV9-hα-GAL variant 1 and rAAV9-hα-GAL-variant 2, as compared to controls. FIG. 12J is a bar graph showing LysoGb3 substrate levels in terminal serum at 2 dosages of rAAV9-hα-GAL variant 1 and rAAV9-hα-GAL-variant 2, as compared to controls. FIG. 12K is a bar graph showing LysoGb3 substrate levels in kidney at 2 dosages of rAAV9-hα-GAL variant 1 and rAAV9-hα-GAL-variant 2, as compared to controls. FIG. 12L is a bar graph showing LysoGb3 substrate levels in heart at 2 dosages of rAAV9-hα-GAL variant 1 and rAAV9-hα-GAL-variant 2, as compared to controls. FIG. 12M is a bar graph showing LysoGb3 substrate levels in liver at 2 dosages of rAAV9-hα-GAL variant 1 and rAAV9-hα-GAL-variant 2, as compared to controls. FIG. 12N is a graph showing vector genome copy in various tissues when treated with 2 dosages of rAAV9-hα-GAL variant 1 and rAAV9-hα-GAL-variant 2, as compared to controls. FIG. 12O is a graph showing mRNA copy in various tissues when treated with 2 dosages of rAAV9-hα-GAL variant 1 and rAAV9-hα-GAL-variant 2, as compared to controls.

FIG. 13A is a graph showing alpha galactosidase activity in serum over 32 weeks in mice after iv injection at two different dosages of rAAV9-hα-GAL variant 1 compared to control. FIG. 13B is a graph showing alpha galactosidase activity in serum of non-human primate when rAAV9-hα-GAL variant 1 is injected iv over 28 days. FIG. 13C is a graph showing alpha galactosidase activity in serum of non-human primate when rAAV9-hα-GAL variant 1 is injected iv over 90 days.

### DEFINITIONS

Approximately or about: As used herein, the term “approximately,” as applied to one or more values of interest, refers to a value that is similar to a stated reference value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). It is understood that when the term “about” or “approximately” is used to modify a stated reference value, the stated reference value itself is covered along with values that are near the stated reference value on either side of the stated reference value.

Administering: The terms “administer”, “administration” and “administering” refer to providing a composition of the present invention (e.g., a recombinant gene therapy vector expressing alpha galactosidase) to a subject in need thereof (e.g., to a person suffering from the effects of Fabry disease).

Administered in combination: As used herein, the term “administered in combination” or “combined administration” means that two or more agents are administered to a subject at the same time or within an interval such that there can be an overlap of an effect of each agent on the patient. In some embodiments, the administrations of the agents are spaced sufficiently closely together such that a combinatorial (e.g., a synergistic) effect is achieved.

Amino acid substitution: The term “amino acid substitution” refers to replacing an amino acid residue present in a parent or reference sequence (e.g., a wild type GLA sequence) with another amino acid residue. An amino acid can be substituted in a parent or reference sequence (e.g., a wild type GLA polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An (YZ), wherein A is the single letter code corresponding to the amino acid residue substituting the amino acid naturally or originally present at position X, and Y and Z are alternative substituting amino acid residues.

The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (C_{α}). In various embodiments described herein, one or more amino acids in the wild-type GLA sequence may be substituted with a different amino acid, thereby resulting in a variant of the α-GAL protein.

Substitutions in a protein or polypeptide amino acid sequence may either be conservative or non-conservative in nature. A conservative amino acid substitution refers to a substitution of a residue with a different residue having a similar side chain, and thus typically involves substitution of the amino acid in a polypeptide (e.g., α-GAL amino acid sequence) with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. A non-conservative substitution refers to substitution of an amino acid in a polypeptide (e.g., α-GAL amino acid sequence) with an amino acid with significantly differing side chain properties. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

In the context of the present disclosure, substitutions (even when they referred to as amino acid substitution) are conducted at the nucleic acid level, i.e., substituting an amino acid residue with an alternative amino acid residue is conducted by substituting the codon encoding the first amino acid with a codon encoding the second amino acid.

AVV vector: As used herein, the term “AAV vector” comprises a capsid protein and a viral genome, wherein the viral genome comprises at least one transgene region and at least one inverted terminal repeat (ITR). The AAV vector and/or its component capsid and viral genome may be engineered to alter tropism to a particular cell-type, tissue, organ or organism. In the context of the present invention, the viral genome comprises a GLA transgene, e.g., a nucleic acid sequence encoding α-GAL or variant thereof.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Blood urea nitrogen: As used herein, the term “Blood urea nitrogen” or “BUN” refers to urea content in blood. Blood urea nitrogen is elevated in pathology associated with kidney. Chronic kidney disease is one of the main features of Fabry disease, causing end-stage renal failure. Gb-3 deposits in glomerular podocytes are thought to contribute at least in part, to the proteinuria or to the rates of progression or severity of kidney involvement in Fabry disease. Blood urea nitrogen measures the efficiency of kidneys to remove urea from the blood. High BUN levels indicate poor renal function.

Chimera: As used herein, “chimera” is an entity having two or more incongruous or heterogeneous parts or regions. For example, a chimeric molecule can comprise a first part comprising a GLA polypeptide, and a second part (e.g., genetically fused to the first part) comprising a second therapeutic protein (e.g., a protein with a distinct enzymatic activity, an antigen binding moiety, or a moiety capable of extending the plasma half-life of α-GAL, for example, an Fc region of an antibody).

Codon substitution: As used herein, the terms “codon substitution” or “codon replacement” in the context of sequence optimization refer to replacing a codon present in a reference nucleic acid sequence with another codon. A codon can be substituted in a reference nucleic acid sequence, for example, via chemical peptide synthesis or through recombinant methods known in the art. Accordingly, references to a “substitution” or “replacement” at a certain location in a nucleic acid sequence (e.g., an mRNA) or within a certain region or subsequence of a nucleic acid sequence (e.g., an mRNA) refer to the substitution of a codon at such location or region with an alternative codon.

Codon optimized: The term “codon optimized” or “codon optimization” refers to changes in the codons of the polynucleotide encoding a protein (e.g., GLA gene) such that the encoded protein is more efficiently expressed, e.g., in a cell or an organism. In some embodiments, the polynucleotides encoding the α-GAL enzymes may be codon optimized for optimal production from the host organism(s) and/or cell type(s) selected for expression accounting for GC content, cryptic splice sites, transcription termination signals, motifs that may affect RNA stability, and nucleic acid secondary structures, as well as any other factors of interest.

Dose: As used herein, the terms “dose” and “dosage” are used interchangeably herein. A dose refers to the amount of active ingredient given to an individual at each administration. The dose will vary depending on a number of factors, including frequency of administration; size and tolerance of the individual; severity of the condition; risk of side effects; and the route of administration. One of skill in the art will recognize that the dose can be modified depending on the above factors or be based on therapeutic progress. The term “dosage form” refers to the particular format of the pharmaceutical, and depends on the route of administration.

Engineered variants: The term “engineered α-GAL variants” or “engineered variants” refers to GAL proteins, where, when compared to the wild-type α-GAL, one or more amino acid residues have been modified by substitution, deletion or insertion. In some embodiments, engineered variants are characterized by improved efficacy and pharmacokinetic profiles, due to, for example, modified the structural attributes of the protein. In some embodiments, engineered α-GAL variants enhance clearing of substrates from tissues, such as, serum, kidney, heart and/or liver. The engineered variants may be synthesized or produced recombinantly.

Gb3: As used herein, the term “Gb3” or “globotriaosylceramide” or “GB3” or “gb3” or “CD77” or “GL-3” refers to a type of glycosphingolipid that accumulates in lysosomes in Fabry disease and is considered to be the main causative metabolite.GB3 is formed by α-linkage of galactose to lactosylceramide catalyzed by A4GALT.GB3 is hydrolyzed at the terminal alpha linkage by GLA. Fabry disease is exemplified by accumulation of GB3 in in all organs (especially the heart and kidneys), as well as many cells and urine. Such accumulation is accompanied by a marked increase in the risk of stroke, heart disease (hypertrophic cardiomyopathy, rhythm and conduction system disorders, coronary artery disease, valve abnormalities, etc.) and chronic proteinuria renal failure. In some embodiments, de-acylatedGB3 or lysoGb3 is also a valuable biomarker for Fabry disease.

Gene: The term “gene,” as used herein, refers to a DNA region encoding a protein or polypeptide (e.g., an alpha-galactosidase enzyme, as described herein), as well as all DNA regions which regulate the production of the protein or polypeptide, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

GLA gene: As used herein, the term “GLA gene” or “galactosidase gene” or “alpha-galactosidase gene” or “α-galactosidase gene” refers to a gene which encodes for the enzyme alpha-galactosidase that breaks down globotriaosylceramide. Genetic mutation in the GLA gene results in defective enzyme function of alpha-galactosidase. In humans, the GLA gene is located at Xq22.1, which is the long (q) arm of the X chromosome at position 22.1. Some of the other names by which the GLA gene may be referred to include AGAL HUMAN, Agalsidase alpha, Alpha-D-galactosidase A, alpha-D-galactosidase galactohydrolase, Alpha-galactosidase, alpha-Galactosidase A, ceramidetrihexosidase, GALA, galactosidase or Melibiase.

Galactosidase: The term “galactosidase” or “alpha galactosidase A” or“ α-galactosidase A” or “α-GAL”, as used herein, refers to the enzyme encoded by the GLA gene. Human alpha galactosidase (EC 3.2.1.22) is a lysosomal enzyme which hydrolyses terminal alpha galactosyl moieties from glycolipids and glycoproteins. As used herein, the term α-GAL may refer to wild-type enzyme or a variant thereof. Deficiency of alpha galactosidase A causes Fabry disease (also referred to as angiokeratoma corporis diffusum, Anderson-Fabry disease, hereditary dystopic lipidosis, alpha-galactosidase A deficiency, α-GAL deficiency, and ceramide trihexosidase deficiency), which is an X-linked inborn error of glycosphingolipid catabolismin various embodiments described herein, a gene therapy platform is provided for treatment of Fabry disease.

“Hypoalgesia or hypalgesia”: As used herein, the term “hypoalgesia” or “hypalgesia” refers to a decreased sensitivity to painful stimuli. Hypoalgesia occurs when nociceptive (painful) stimuli are interrupted or decreased somewhere along the path between the input (nociceptors), and the places where they are processed and recognized as pain in the conscious mind. Hypoalgesic effects can be mild, such as massaging a stubbed toe to make it hurt less or taking aspirin to decrease a headache, or they can be severe, like being under strong anesthesia.

“Improved enzyme property”: The term “improved enzyme property” refers to any property or attribute of an engineered α-GAL polypeptide that is an improvement relative to the same property or attribute of a reference α-GAL polypeptide (e.g., as compared to a wild-type α-GAL polypeptide or another engineered α-GAL polypeptide). Improved properties include, but are not limited to such properties as increased gene expression, increased protein production, increased thermoactivity, increased thermostability, increased activity at various pH levels, increased stability, increased enzymatic activity, increased substrate specificity or affinity, increased specific activity, increased resistance to substrate and/or product inhibition, increased chemical stability, improved chemoselectivity, improved solvent stability, increased tolerance to acidic, neutral, or basic pH, increased tolerance to proteolytic activity (i.e., reduced sensitivity to proteolysis), reduced aggregation, increased solubility, reduced immunogenicity, improved post-translational modification (e.g., glycosylation), altered temperature profile, increased cellular uptake, increased lysosomal stability, increased ability to deplete cells of GB3, increased secretion from α-GAL producing cells, etc. In various embodiments, the gene therapy vectors encompassed by the present disclosure comprise a nucleic acid sequence encoding an α-GAL polypeptide comprising one or more improved enzyme properties relative to a reference α-GAL polypeptide. In some embodiments, the nucleic acid sequence encoding a α-GAL polypeptide exhibiting one or more improved enzyme properties, is codon optimized.

In various embodiments, codon optimized and/or engineered α-GAL variants exhibit one or more aforementioned improved properties. In a particular embodiment, α-GAL variant has improved serum and lysosomal stability and in other embodiments, α-GAL variant has increased specific catalytic activity over wild type α-GAL polypeptide.

“Increased enzymatic activity”: The term “increased enzymatic activity” refers to an increase in specific activity (e.g., product produced/time/weight protein) or an increase in percent conversion of the substrate to the product (e.g., percent conversion of starting amount of substrate to product in a specified time period) using a specified amount of an engineered α-GAL enzyme as compared to a reference α-GAL enzyme (e.g., a wild type α-GAL enzyme or another engineered variant). Any suitable method known in the art and/or those described herein may be used to determine enzyme activity. Any property relating to enzyme activity may be affected, including the classical enzyme properties of Kₘ, Vₘₐₓ or K_{cat}, changes of which can lead to increased enzymatic activity. Improvements in enzyme activity can be from about 1.1 fold the enzymatic activity of the corresponding wild-type enzyme, to as much as 2-fold, 5-fold, 10-fold, 20-fold, 25-fold, 50-fold, 75-fold, 100-fold, 150-fold, 200-fold or more enzymatic activity than a reference α-GAL enzyme.

Intrinsic Expression: The term “intrinsic expression” and grammatical equivalents thereof refers to expression of a gene within one or more cells into which a transgene is introduced. Intrinsic expression uses the cell's own or pre-existing transcription or translation mechanisms and resources for expression of the transgene. For example, in some embodiments, when this term is used to refer to an “intrinsic α-GAL expression system” it means that α-GAL is expressed from within the cells of a tissue.

Nucleic acid: As used herein, the terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

Neuropathy: As used herein, the term “neuropathy” refers to damages and/or dysfunctions of one or more nerves. A “peripheral neuropathy” refers to any damages and/or dysfunctions that affect the peripheral nervous system (the PNS). “Peripheral neuropathy” can manifest as one or a combination of motor, sensory, sensorimotor, or autonomic neural dysfunction. Peripheral neuropathy has been shown to be associated with systemic diseases, including Fabry disease. In the context of the present disclosure, peripheral neuropathy associated with Fabry disease mostly manifests as sensory dysfunctions (e.g., thermal hyposensitivity). One variety of peripheral neuropathy is “demyelinating peripheral neuropathy”, a broad class of peripheral neuropathies that are associated with the destruction or removal of myelin, the lipid-rich sheath surrounding and insulating nerve fibers, from nerves.

Operative linkage: As used herein, the terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

Physiological pH: As used herein, “physiological pH” means the pH range generally found in a subject's (e.g., human) blood, that is pH 7.4.

Basic pH: The term “basic pH” (e.g., used with reference to improved stability at basic pH conditions or increased tolerance to basic pH) means a pH range of about 7 to 11.

Acidic pH: The term “acidic pH” (e.g., used with reference to improved stability to acidic pH conditions or increased tolerance to acidic pH) means a pH range of about 1.5 to 6. Polypeptide: As used herein, the terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

Promoter: As used herein, the term “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. In some embodiments, liver-specific promoters include, for example, transthyretin promoter (TTR); thyroxine-binding globulin (TBG) promoter; hybrid liver-specific promoter (HLP), and alpha-1-antitrypsin (AAT) promoter. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

Sequence Optimization: As used herein, the term “sequence optimization” refers to a process or series of processes by which nucleobases in a reference nucleic acid sequence are replaced with alternative nucleobases, resulting in a nucleic acid sequence with improved properties, e.g., improved protein expression or increased activity.

The terms “individual,” “subject,” “subject in need thereof” and “patient,” used interchangeably herein, refer to a mammal, including, but not limited to, murines, simians, humans, mammalian farm animals, mammalian sport animals, and mammalian pets. In preferred embodiments, the individual is a human. In various embodiments, a subject or a subject in need thereof is a Fabry patient exhibiting one or more symptoms associated with peripheral neuropathy. Therapeutically effective: A “therapeutically effective” amount or dose or “sufficient/effective” amount or dose, is a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

Treatment: As used herein, the term “treatment” or “therapy” generally means obtaining a desired physiologic effect. The effect may be prophylactic in terms of completely or partially preventing a disease or condition or symptom thereof and/or may be therapeutic in terms of a partial or complete cure for an injury, disease or condition and/or amelioration of an adverse effect attributable to the injury, disease or condition and includes arresting the development or causing regression of a disease or condition. Treatment can also include prophylactic use to mitigate the effects of injury, should it occur. For example, in one aspect, the present invention includes pre-administration to mitigate damage prior to surgery involving the peripheral nervous system. Treatment can also refer to any delay in onset, amelioration of symptoms, improvement in patient survival, increase in survival time or rate, etc. The effect of treatment can be compared to an individual or pool of individuals not receiving the treatment.

Tropism: As used herein, the terms “tropism,” or “tropicity” in the context of AAV refers to AAV capsid serotype having varying transduction profiles for different tissue types. In some embodiments, “systemic tropism” and “systemic transduction” (and equivalent terms) indicate that the virus capsid or virus vector of the invention exhibits tropism for or transduces, respectively, more than one tissue, or multiple tissues or organs throughout the body (e.g., more than one of brain, lung, skeletal muscle, heart, liver, kidney and/or pancreas).

Thermal hypesthesia: As used herein, the term “thermal hypesthesia” or “thermal dysesthesia” or “thermal hypoesthesia” refers to an insensitive or less sensitive to a heat stimuli.

Vector: As used herein, the term “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. In some embodiments, the vector is a virus, which includes, for example, encapsulated forms of vector nucleic acids, and viral particles in which the vector nucleic acids have been packaged. In some embodiments, the vector is not a wild-type strain of a virus, in as much as it comprises human-made mutations or modifications. In some embodiments, the vector is derived from a wild-type viral strain by genetic manipulation (i.e., by deletion) to comprise a conditionally replicating virus, as further described herein. In some embodiments, the vector is delivered by non-viral means. In some embodiments, vectors described herein are gene therapy vectors, which are used as carriers for delivery of polynucleotide sequences (e.g., an alpha galactosidase enzyme) to cells. In a particular embodiment, a gene therapy vector described herein is a recombinant AAV vector (e.g., AAV8 or AAV9).

Wild-type: As used herein, the term “wild-type” and “naturally-occurring” refer to the form of a nucleic acid or protein found in nature. For example, a wild-type polypeptide or polynucleotide sequence is a sequence present in an organism that can be isolated from a source in nature and which has not been intentionally modified by human manipulation.

The recitation of numerical ranges by endpoints herein includes all numbers and fractions subsumed within that range (e.g. 1 to 5 includes 1, 1.5, 2, 2.75, 3, 3.9, 4 and 5). It is also to be understood that all numbers and fractions thereof are presumed to be modified by the term “about.”

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

### DETAILED DESCRIPTION

The present disclosure relates to rAAV based gene therapy for treatment and/or prevention of Fabry disease, in particular, for ameliorating and/or preventing or treating neuropathy in Fabry disease. The present disclosure provides, among other things, (1) an intrinsic GLA expression system in tissues affected by Fabry disease, (2) methods to achieve sustained and high expression of α-galactosidase (α-GAL) to reduce disease burden and treatment burden associated with progression of Fabry disease and (3) use of a rAAV vector encoding GLA to achieve amelioration of Fabry disease associated phenotypes, in particular, peripheral neuropathy in Fabry disease.

In some embodiments, the gene therapy treatment described herein utilizes a rAAV vector with broad tissue tropism which comprises a ubiquitous promoter to drive widespread gene expression, resulting in sustained high levels of protein expression and robust protein exposure to a wide range of tissues and/or decrease in GB3 or lysoGb3 levels. In addition, the GLA transgene used in the present application is codon optimized and/or expresses an engineered variant of α-GAL, resulting in an increase in α-GAL activity in vivo, and/or a decrease in lysoGb3 or Gb3 levels in vivo. Additionally, the present gene approach to drive expression of GLA variants that encode α-GAL protein with increased half-life and improved cellular uptake provides further increases in α-GAL exposure in key target tissues, e.g., the nervous tissues, allowing better treatment outcomes of Fabry disease, for example, for improved peripheral neuropathy outcomes.

The methods and compositions provided herein can be used to achieve sustained expression of GLA in a wide variety of tissues that are affected in Fabry disease. Thus, the present application provides composition and methods that are highly effective in the treatment of Fabry disease and alleviation of associated symptoms such as peripheral neuropathy.

### α-galactosidase and Fabry Disease

Fabry disease is an X-linked inherited disease caused by aberrant lysosomal hydrolase, α-galactosidase A (α-GAL), due to mutations in the GLA gene. Because α-GAL is necessary for catabolismof glycolipids, such as sphingolipids, deficiency or malfunction of α-GAL causes accumulation of sphingolipids in tissues.

Fabry disease is a systematic metabolic disease, affecting a number of tissues and organs including kidney, heart, lung and the nervous system. The neurological manifestations of Fabry disease include the peripheral nervous system (PNS) involvement, with globotriaosylceramide accumulation found in Schwann cells and dorsal root ganglia. Involvement of the peripheral nervous system affects mainly small Aδ and C fibers, and is probably causally related to the altered autonomic function and neuropathic pain found in Fabry disease. Other related neurological problems include hypohidrosis and other abnormalities associated with nervous system dysfunction.

The peripheral neuropathy in Fabry disease manifests as neuropathic pain, reduced cold and warm sensation (i.e., thermal hypoesthesia), hearing loss, other sensory deficiency and possibly, gastrointestinal disturbances.

Patients with Fabry disease begin having pain towards the end of the first decade of life or during puberty (Ries et al, Pediatric Fabry disease, Pediatrics, 2005; 115: e344-355). In general, neuropathic pain in Fabry disease can be continuous (i.e., chronic) or consist of episodic attacks brought about by changes in body or ambient temperature, as well as other stressful situations (MacDermot and MacDermot, Neuropathic pain in Anderson-Fabry disease: pathology and therapeutic options. Eur J Pharmacol. 2001; 429:121-125). Episodic pain in Fabry disease, termed “Fabry crises,” typically begins in the extremities and radiates proximally, and may be triggered by exercise, illness, temperature changes, or other physical and emotional stresses. This neuropathic pain is also associated with a lack of temperature perception.

The neuropathy of Fabry disease is associated with significantly increased cold and warm detection thresholds (i.e., thermal hypoesthesia or thermal hyposensitivity) in the hands and feet (Luciano et al. Physiological characterization of neuropathy in Fabry's disease. Muscle Nerve. 2002; 26:622-629; and Dutsch et al. Small fiber dysfunction predominates in Fabry neuropathy. J Clin Neurophysiol. 2002; 19:575-586). A well-established biophysical quantitative sensory testing technique can be used to measure those thresholds (Dyck et al., A 4, 2, and 1 stepping algorithm for quick and accurate estimation of cutaneous sensation threshold. Neurology. 1993; 43:1508-1512).

Increased perception thresholds to warm and cold temperatures (hypoesthesia) in males and carrier females were shown to initiate with burning pain and acute discomfort (Hilz, et al.,. Lower limb cold exposure induces pain and prolonged small fiber dysfunction in Fabry patients. Pain, 2000; 84:361-365). It has been reported that human patients have reduced number of small diameter myelinated nerve fibers (Onishi and Dyck, Loss of small peripheral sensory neurons in Fabry disease. Histologic and morphometric evaluation of cutaneous nerves, spinal ganglia, and posterior columns. Arch. Neurol. 1974; 31:120-127). Studies concerning nerve damage leading to pain, based on post-mortem characterization of tissues from few Fabry disease patients, showed a significant reduction of small, thinly myelinated and unmyelinated nerve fibers (Hilz, et al., Enzyme replacement therapy improves function of C-, Adelta-, and Abeta-nerve fibers in Fabry neuropathy. Neurology; 2004; 62:1066-1072).

Several studies indicate that Fabry disease is associated with peripheral neuropathy affecting predominantly the small myelinated (Aδ) fibers and unmyelinated (C) fibers. The potential causal mechanisms may be due to ischaemia of nerves caused by glycolipid accumulation in the vasa nervorum or an intrinsic nerve dysfunction (Luciano et al. Physiological characterization of neuropathy in Fabry's disease. Muscle Nerve. 2002; 26:622-629; Hilz et al. Lower limb cold exposure induces pain and prolonged small fiber dysfunction in Fabry patients. Pain. 2000; 84:361-365; and Gadoth and Sandbank. Involvement of dorsal root ganglia in Fabry's disease. J Med Genet. 1983; 20:309-312).

Sensory nerve fiber neuropathy (SFSN) in Fabry patients was found to be associated with sensorineural hearing loss (Ries et al., Neuropathic and cerebrovascular correlates of hearing loss in Fabry disease. Brain 2007, 130:143-150).

Other neuropathy in Fabry disease includes that some patients have impaired vibration thresholds and nerve conduction abnormality. Pathological examination of peripheral nerves (such as the sural nerve) typically shows a normal number of large myelinated fibers, but there is significant loss of unmyelinated small fibers. The glycolipid deposits in sensory ganglia have been associated with the peripheral neuropathy. In addition, this neuropathy in Fabry disease is associated with severe loss of intra-epidermal innervation.

It has been shown that in the Fabry knockout mice the abnormal accumulation of Gb3 due to the deficiency of lysosomal α-galactosidase activity is associated with abnormalities of morphology of the sciatic nerve, reduced number of unmyelinated axons and small myelinated axons and preserved large diameter myelinated axons, behavior and sensitivity to heat stimuli, displaying similar features as the ones described for human patients (Rodrigues et al., Neurophysiological, behavioral and morphological abnormalities in the Fabry knockout mice; Neurobiol. Dis., 2009, 33(1): 48-56). Fabry knockout mice can be a possible explanation for the alterations observed in thermal sensibility (hypoalgesia).

In summary, it is well established that GB3 inclusions are present in DRG neurons of both patients and rodent preclinical models and these inclusions lead to cell swelling, including increases in DRG neuron soma diameter and total DRG volume. GB3 content can be more than 10-times higher in the DRG than in regions of the brain (e.g., Kaye et al., Nervous system involvement in Fabry's disease: clinicopathological and biochemical correlation. Ann Neurol 1988; 23:505-509); these high levels of Gb3 can result in DRG neuron stress and death. In addition to DRG neuron cell bodies, lipid inclusions are prominent in the axons of peripheral nerves and correlate with axon morphological abnormalities including irregular-shaped axons, enlarged axons, and nonuniform myelin. This dramatic small fiber loss is most prominent in the distal long axons of the lower extremities. There can also be a decline in the innervation of proximal sites such as the thigh, but the loss is most pronounced around the feet. Studies suggest that the fiber loss is substantially higher in the skin than in the peripheral nerve trunk.

Current treatment options for Fabry disease include recombinant enzyme replacement therapies (ERTs). ERTs slow the progression of the Fabry disease but do not completely halt or reverse the disease. Current treatment for Fabry disease predominantly achieves a slowing of disease progression limited to kidney and heart, with inadequate or no improvements in other organs/tissues, especially in the nervous system. Fabry patients also require continuous protein-based infusion, sometimes resulting in infusion reactions and augmented immunogenicity. Such continuous disease management requirements also increase the “treatment burden” or the added and ongoing workload (i.e. necessities and demands) for patients in order for them to adhere to recommendations made by their clinicians to manage their morbidity and wellbeing. In severely affected classical male Fabry disease patients, yearly loss of renal function despite treatment is up to −6.82 mL/min/1.73 m²/year (Germain et al, J Med Genet. 2015 May; 52(5):353-8.2015), as compared to healthy subjects, who have a yearly loss of renal function of −1 mL/min/1.73 m²/year. These needs could be addressed by vector delivery of GLA as described herein.

Gene therapy is a promising approach for the treatment and prevention of Fabry disease. One advantage of the gene therapy approach to the treatment of Fabry disease is continuous α-GAL exposure rather than an intermittent α-GAL exposure provided by ERT infusion. The gene therapy approach can potentially allow for uptake by certain tissues and cell types (e.g., peripheral nervous system) that is not easily achieved by infused ERTs. The constant availability of α-GAL in the lysosomes can prevent glycosphingolipid re-accumulation between doses. The significant enhancement in enzyme distribution to target cells could provide a transformative therapy with the possibility of achieving superior clinical benefit over current therapies. In addition, gene therapy accompanied by hepatocyte transduction can harness the tolerogenic nature of the liver and induce systemic immunological tolerance to transgene product eliminating the risk of reduced treatment efficacy due to anti-drug antibodies. Without wishing to be bound by theory, it is believed that these benefits, combined with a single long-lasting dose, could both address the need for a treatment with a significantly higher treatment efficacy and reduce treatment burden on patients and caregivers.

As discussed below, the present disclosure provides GLA gene therapy for treating and/or ameliorating one or more neuropathic manifestations in Fabry disease.

### Gene Therapy Vectors

In one aspect of the present disclosure, a GLA expression system (i.e., a GLA transgene) comprises a viral vector comprising a polynucleotide encoding α-GAL or variant thereof, controlled by a ubiquitous promoter is provided. In some aspects, the viral vector a recombinant AAV vector. In particular, the recombinant AAV (rAAV) vector has a broad tissue tropism. The rAAV vector contemplated in the present application has broad tissue tropism and utilizes a ubiquitous promoter to drive widespread gene expression, resulting in sustained high levels of protein expression and robust protein exposure to a wide range of tissues and/or decrease in GB3 or lysoGb3 levels. In addition, the GLA transgene is codon optimized and/or expresses an engineered variant of α-GAL, resulting in an increase in α-GAL activity in vivo, and/or a decrease in lysoGb3 or GB3 levels in vivo.

### α-GAL Enzyme and Variants

The viral vectors described herein comprise a polynucleotide encoding α-GAL enzyme or variant thereof. In some embodiments, the α-GAL enzyme is a naturally occurring (wild-type) enzyme having an amino acid sequence of SEQ ID NO: 1 or SEQ ID NO: 5. In some embodiments, the α-GAL enzyme is a functional variant, such as a modified α-GAL enzyme.

Exemplary amino acid sequences of α-GAL enzyme and its variants contemplated for use in the vectors of the present disclosure are provided in the Table 1 below.

TABLE 1
Amino acid sequences of α - GAL enzyme and variants thereof

| Description | Sequence (5′-3′) |
|---|---|
| WT | LDNGLARTPTMGWLHWERFMCNLDCQEEPDSCISEKLFMEMAELMVSEGWKDAGYEYLCIDD |
| | CWMAPQRDSEGRLQADPQRFPHGIRQLANYVHSKGLKLGIYADVGNKTCAGFPGSFGYYDIDA |
| | QTFADWGVDLLKFDGCYCDSLENLADGYKHMSLALNRTGRSIVYSCEWPLYMWPFQKPNYTEI |
| | RQYCNHWRNFADIDDSWKSIKSILDWTSFNQERIVDVAGPGGWNDPDMLVIGNFGLSWNQQ |
| | VTQMALWAIMAAPLFMSNDLRHISPQAKALLQDKDVIAINQDPLGKQGYQLRQGDNFEVWER |
| | PLSGLAWAVAMINRQEIGGPRSYTIAVASLGKGVACNPACFITQLLPVKRKLGFYEWTSRLRSHIN |
| | PTGTVLLQLENTMQMSLKDLL (SEQ ID NO: 1) |
| Variant 1 | LDNGLARTPPMGWLHWERFMCNLDCQEEPDSCISEKLFEEMAERMVTDGWKDAGYEYLCIDD |
| | CWMAPQRDSEGRLQADPQRFPHGIRQLANHVHSKGLKLGIYADVGNKTCAGFPGSFGYYDIDA |
| | QTFADWGVDLLKFDGCYCDSLENLADGYKHMSLALNKTGRPIVYSCEWPLYMWPFQKPNYTEI |
| | RQYCNHWRNFADIDDSWASIKSILDWTSRNQERIVDVAGPGGWNDPDMLVIGNFGLSWDQQ |
| | VTQMALWAIMAGPLFMSNDLRAISPQAKALLQDKDVIAINQDPLGKQGYQLRKGDNFEVWER |
| | PLSGDAWAVAIINRQEIGGPRGYTIPVASLGKGVACNPACFITQLLPVKRKLGFYEATSRLRSHINP |
| | TGTVLLQLENTMQTSLKDLL(SEQ ID NO: 2) |
| Variant 2 | LDNGLARTPTMGWLHWERFMCNLDCQEEPDSCISEKLFMEMAERMVSEGWKDAGYEYLCID |
| | DCWMAPQRDSEGRLOADPQRFPHGIRQLANYVHSKGLKLGIYADVGNKTCAGFPGSFGYYDID |
| | AQTFADWGVDLLKFDGCYCDSLENLADGYKHMSLALNRTGRSIVYSCEWPLYMWPFQKPNYTE |
| | IRQYCNHWRNFADIDDSWASIKSILDWTSRNQERIVDVAGPGGWNDPDMLVIGNFGLSWDQ |
| | QVTQMASWAIMAAPLFMSNDLRHISPQAKALLQDKDVIAINQDPLGKQGYQLRKGDNFEVWE |
| | RPLSGDAWAVAIINRQEIGGPRSYTIPVASLGKGVACNPACFITQLLPVKRQLGFYNWTSRLKSHI |
| | NPTGTVLLQLENTMQMSLKDLL (SEQ ID NO: 3) |
| Signal | MQLRNPELHLGCALALRFLALVSWDIPGARA (SEQ ID NO: 4) |
| peptide | |
| Wild type α- | MQLRNPELHLGCALALRFLALVSWDIPGARALDNGLARTPTMGWLHWERFMCNLDCQEEPDS |
| GAL with | CISEKLFMEMAELMVSEGWKDAGYEYLCIDDCWMAPQRDSEGRLQADPQRFPHGIRQLANYV |
| signal | HSKGLKLGIYADVGNKTCAGFPGSFGYYDIDAQTFADWGVDLLKFDGCYCDSLENLADGYKHMS |
| peptide | LALNRTGRSIVYSCEWPLYMWPFQKPNYTEIRQYCNHWRNFADIDDSWKSIKSILDWTSFNQER |
| | IVDVAGPGGWNDPDMLVIGNFGLSWNQQVTQMALWAIMAAPLFMSNDLRHISPQAKALLQ |
| | DKDVIAINQDPLGKQGYQLRQGDNFEVWERPLSGLAWAVAMINRQEIGGPRSYTIAVASLGKG |
| | VACNPACFITQLLPVKRKLGFYEWTSRLRSHINPTGTVLLQLENTMQMSLKDLL (SEQ ID NO: 5) |
| Variant 1 | MQLRNPELHLGCALALRFLALVSWDIPGARALDNGLARTPPMGWLHWERFMCNLDCQEEPDS |
| with signal | CISEKLFEEMAERMVTDGWKDAGYEYLCIDDCWMAPQRDSEGRLQADPQRFPHGIRQLANHV |
| peptide | HSKGLKLGIYADVGNKTCAGFPGSFGYYDIDAQTFADWGVDLLKFDGCYCDSLENLADGYKHMS |
| | LALNKTGRPIVYSCEWPLYMWPFQKPNYTEIRQYCNHWRNFADIDDSWASIKSILDWTSRNQE |
| | RIVDVAGPGGWNDPDMLVIGNFGLSWDQQVTQMALWAIMAGPLFMSNDLRAISPQAKALLQ |
| | DKDVIAINQDPLGKQGYQLRKGDNFEVWERPLSGDAWAVAIINRQEIGGPRGYTIPVASLGKGV |
| | ACNPACFITQLLPVKRKLGFYEATSRLRSHINPTGTVLLQLENTMOTSLKDLL (SEQ ID NO: 6) |
| Variant 2 | MQLRNPELHLGCALALRFLALVSWDIPGARALDNGLARTPTMGWLHWERFMCNLDCQEEPDS |
| with signal | CISEKLFMEMAERMVSEGWKDAGYEYLCIDDCWMAPQRDSEGRLQADPQRFPHGIRQLANYV |
| peptide | HSKGLKLGIYADVGNKTCAGFPGSFGYYDIDAQTFADWGVDLLKFDGCYCDSLENLADGYKHMS |
| | LALNRTGRSIVYSCEWPLYMWPFQKPNYTEIRQYCNHWRNFADIDDSWASIKSILDWTSRNQER |
| | IVDVAGPGGWNDPDMLVIGNFGLSWDQQVTQMASWAIMAAPLFMSNDLRHISPQAKALLQ |
| | DKDVIAINQDPLGKQGYQLRKGDNFEVWERPLSGDAWAVAIINRQEIGGPRSYTIPVASLGKGV |
| | ACNPACFITQLLPVKRQLGFYNWTSRLKSHINPTGTVLLQLENTMQMSLKDLL (SEQ ID NO: 7) |

In some embodiments, an α-GAL enzyme encoded by a GLA transgene comprises a signal peptide sequence MQLRNPELHLGCALALRFLALVSWDIPGARA (SEQ ID NO: 4). In some embodiments, an α-GAL encoded by a GLA transgene comprises a signal peptide sequence at the N-terminus. In some embodiments, an α-GAL enzyme encoded by a GLA transgene comprises a signal peptide sequence at the C-terminus. In some embodiments, an α-GAL enzyme encoded by a GLA transgene comprises SEQ ID NO: 4 at the N-terminus. As non-limiting examples, the α-GAL enzyme comprising a signal peptide comprises an amino acid sequence selected from SEQ ID NO.: 5-7 (Table 1).

In some embodiments, the modified sequence comprises between 1 and 25, 5 and 25, 5 and 20, 5 and 15, 5 and 10, 10 and 25, 10 and 20, 10 and 15, or 15 and 25 amino acid substitutions in comparison to wild-type α-GAL enzyme (SEQ ID NO: 5).

In some embodiments, the modified sequence comprises between 1 and 10 amino acid substitutions in comparison to wild-type α-GAL enzyme (SEQ ID NO: 5). For example, in some embodiments, the modified sequence comprises between 1 and 9 amino acid substitutions in comparison to wild-type α-GAL enzyme (SEQ ID NO: 5). In some embodiments, the modified sequence comprises between 1 and 8 amino acid substitutions in comparison to wild-type α-GAL enzyme (SEQ ID NO: 5). In some embodiments, the modified sequence comprises between 1 and 7 amino acid substitutions in comparison to wild-type α-GAL enzyme (SEQ ID NO: 5). In some embodiments, the modified sequence comprises between 1 and 6 amino acid substitutions in comparison to wild-type α-GAL enzyme (SEQ ID NO: 5). In some embodiments, the modified sequence comprises between 1 and 5 amino acid substitutions in comparison to wild-type α-GAL enzyme (SEQ ID NO: 5). In some embodiments, the modified sequence comprises between 1 and 4 amino acid substitutions in comparison to wild-type α-GAL enzyme (SEQ ID NO: 1). In some embodiments, the modified sequence comprises between 1 and 3 amino acid substitutions in comparison to wild-type α-GAL enzyme (SEQ ID NO: 5).

In some embodiments, the modified sequence comprises 10 amino acid substitutions in comparison to wild-type α-GAL enzyme (SEQ ID NO: 5).

In some embodiments, the GLA transgene expresses a recombinant α-GAL. The recombinant α-GAL comprises a polypeptide sequence having at least 85%, 86%, 87%, 88%, 89%, 90%, 91%, 92%, 93%, 94%, 95%, 96%, 97%, 98%, 99%, or more sequence identity to SEQ ID NO: 5, or a functional fragment thereof. In some embodiments, the recombinant α-GAL comprises a polypeptide sequence having at least 85% sequence identity to SEQ ID NO: 5. In some embodiments, the recombinant α-GAL comprises a polypeptide sequence having at least 86% sequence identity to SEQ ID NO: 5. In some embodiments, the recombinant α-GAL comprises a polypeptide sequence having at least 87% sequence identity to SEQ ID NO: 5. In some embodiments, the recombinant α-GAL comprises a polypeptide sequence having at least 88% sequence identity to SEQ ID NO: 5. In some embodiments, the recombinant α-GAL comprises a polypeptide sequence having at least 89% sequence identity to SEQ ID NO: 5. In some embodiments, the recombinant α-GAL comprises a polypeptide sequence having at least 90% sequence identity to SEQ ID NO: 5. In some embodiments, the recombinant α-GAL comprises a polypeptide sequence having at least 91% sequence identity to SEQ ID NO: 5. In some embodiments, the recombinant α-GAL comprises a polypeptide sequence having at least 92% sequence identity to SEQ ID NO: 5. In some embodiments, the recombinant α-GAL comprises a polypeptide sequence having at least 93% sequence identity to SEQ ID NO: 5. In some embodiments, the recombinant α-GAL comprises a polypeptide sequence having at least 94% sequence identity to SEQ ID NO: 5. In some embodiments, the recombinant α-GAL comprises a polypeptide sequence having at least 95% sequence identity to SEQ ID NO: 5. In some embodiments, the recombinant α-GAL comprises a polypeptide sequence having at least 96% sequence identity to SEQ ID NO: 5. In some embodiments, the recombinant α-GAL comprises a polypeptide sequence having at least 97% sequence identity to SEQ ID NO: 5. In some embodiments, the recombinant α-GAL comprises a polypeptide sequence having at least 98% sequence identity to SEQ ID NO: 5. In some embodiments, the recombinant α-GAL comprises a polypeptide sequence having at least 99% sequence identity to SEQ ID NO: 5.

In some embodiments, the recombinant α-GAL comprises at least one substitution or substitution in SEQ ID NO:5 at one or more positions selected from: T41/M70/L75/S78/E79/Y123/R193/S197/K237/F248/N247/N278/L286/A292/H302/Q333/K314/L347/M353/S364/A368/S371/K374/K393/F396/E398/W399/R404/M423.

Additional exemplary GLA transgene and α-GAL enzyme sequences can be found in PCT publication Nos: PCT/US2021/019811, PCT/US2019/067493 and PCT/US2015/063329, each of which is incorporated by reference herein, in its entirety.

In some embodiments, the modified α-GAL enzyme has increased stability (e.g., serum stability), intracellular stability (e.g., lysosomal activity), and/or specific catalytic activity in comparison to wild-type α-GAL enzyme (SEQ ID NO: 5).

In some embodiments, the present disclosure encompasses a gene therapy vector comprising a GLA sequence having 70%, 75%, 80%, 85%, 90%, 95%, 96%, 97%, 98%, 99%, or more than 99% identity to any one of SEQ ID NOs: 1-3 and 5-7. In some embodiments, the vector comprises a GLA sequence having between 70% and 100% identity to SEQ ID NOs. 1-3 and 5-7. In some embodiments, the vector comprises a GLA sequence having between 75% and 100% identity to SEQ ID NOs. 1-3 and 5-7. In some embodiments, the vector comprises a GLA sequence having between 80% and 100% identity to SEQ ID NOs. 1-3 and 5-7. In some embodiments, the vector comprises a GLA sequence having between 85% and 100% identity to SEQ ID Nos: 1-3 and 5-7. In some embodiments, the vector comprises a GLA sequence having between 90% and 100% identity to SEQ ID Nos: 1-3 and 5-7. In some embodiments, the vector comprises a GLA sequence having between 95% and 100% identity to SEQ ID Nos: 1-3 and 5-7. In some embodiments, the vector comprises a GLA sequence at least 70% identity to one of SEQ ID Nos: 1-3 and 5-7. In some embodiments, the vector comprises a GLA sequence at least 75% identity to one of SEQ ID Nos: 1-3 and 5-7. In some embodiments, the vector comprises a GLA sequence at least 80% identity to one of SEQ ID Nos: 1-3 and 5-7. In some embodiments, the vector comprises a GLA sequence at least 85% identity to one of SEQ ID Nos: 1-3 and 5-7. In some embodiments, the vector comprises a GLA sequence at least 90% identity to one of SEQ ID Nos: 1-3 and 5-7. In some embodiments, the vector comprises a GLA sequence at least 91% identity to one of SEQ ID Nos: 1-3 and 5-7. In some embodiments, the vector comprises a GLA sequence at least 92% identity to one of SEQ ID Nos: 1-3 and 5-7. In some embodiments, the vector comprises a GLA sequence at least 93% identity to one of SEQ ID Nos: 1-3 and 5-7. In some embodiments, the vector comprises a GLA sequence at least 94% identity to one of SEQ ID Nos: 1-3 and 5-7. In some embodiments, the vector comprises a GLA sequence at least 95% identity to one of SEQ ID Nos: 1-3 and 5-7. In some embodiments, the vector comprises a GLA sequence at least 96% identity to one of SEQ ID Nos: 1-3 and 5-7. In some embodiments, the vector comprises a GLA sequence at least 97% identity to one of SEQ ID Nos: 1-3 and 5-7. In some embodiments, the vector comprises a GLA sequence at least 98% identity to one of SEQ ID Nos: 1-3 and 5-7. In some embodiments, the vector comprises a GLA sequence at least 99% identity to one of SEQ ID Nos: 1-3 and 5-7. In some embodiments, the vector comprises a GLA sequence 100% identity to one of SEQ ID Nos: 1-3 and 5-7.

In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 2. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 3. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 6. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 7.

In some embodiments, the GLA gene therapy vector comprises a polynucleotide encoding an α-GAL enzyme or a variant thereof. In some embodiments, the present disclosure encompasses a gene therapy vector comprising a polynucleotide encoding α-GAL enzyme comprising 70%, 75%, 80%, 85%, 90%, 95%, 96%, 97%, 98%, 99%, or more than 99% identity to SEQ ID NO: 1. In some embodiments, the present disclosure encompasses a gene therapy vector comprising a polynucleotide encoding α-GAL enzyme comprising 70%, 75%, 80%, 85%, 90%, 95%, 96%, 97%, 98%, 99%, or more than 99% identity to SEQ ID NO: 2. In some embodiments, the present disclosure encompasses a gene therapy vector comprising a polynucleotide encoding α-GAL enzyme comprising 70%, 75%, 80%, 85%, 90%, 95%, 96%, 97%, 98%, 99%, or more than 99% identity to SEQ ID NO: 3. In some embodiments, the present disclosure encompasses a gene therapy vector comprising a polynucleotide encoding α-GAL enzyme comprising 70%, 75%, 80%, 85%, 90%, 95%, 96%, 97%, 98%, 99%, or more than 99% identity to SEQ ID NO: 5. In some embodiments, the present disclosure encompasses a gene therapy vector comprising a polynucleotide encoding α-GAL enzyme comprising 70%, 75%, 80%, 85%, 90%, 95%, 96%, 97%, 98%, 99%, or more than 99% identity to SEQ ID NO: 6. In some embodiments, the present disclosure encompasses a gene therapy vector comprising a polynucleotide encoding α-GAL enzyme comprising 70%, 75%, 80%, 85%, 90%, 95%, 96%, 97%, 98%, 99%, or more than 99% identity to SEQ ID NO: 7.

In other embodiments, the GLA transgene of the present disclosure may comprises an α-GAL or variant thereof disclosed in the applicant's previous PCT application NO: PCT/US22/17998 (e.g., sequences in Table 1 of PCT/US22/17998); the contents of which are incorporated herein by reference in their entirety.

In some embodiments, the GLA transgene comprises a nucleic acid sequence encoding a signal peptide sequence 5′atgcagctgaggaacccagaactacatctgggctgcgcgcttgcgcttcgcttcctggccctcgtttcctgggacatccctggggct agagca 3′ (SEQ ID NO: 11). In some embodiments, a GLA sequence comprises a signal peptide sequence at the 5′ end. In some embodiments, a GLA sequence comprises a signal peptide sequence at the 3′ end. In some embodiments, a GLA sequence comprises SEQ ID NO: 11 at the 5′ end. In some embodiments, a GLA sequence comprises SEQ ID NO: 11 at the 3′ end.

In some embodiments, the polynucleotide encoding α-GAL enzyme or variant thereof comprises at least one chemical modification. In some embodiments, the polynucleotide encoding α-GAL enzyme or variant thereof is codon optimized.

As Non-limiting examples, the polynucleotide comprises a nucleotide sequence in Table 2. In some embodiment, the polynucleotide encoding the α-GAL enzyme comprises a nucleotide sequence of SEQ ID NO: 8. In some embodiment, the polynucleotide encoding the α-GAL enzyme comprises a nucleotide sequence of SEQ ID NO: 9. In some embodiment, the polynucleotide encoding the α-GAL enzyme comprises a nucleotide sequence of SEQ ID NO: 10. In some embodiment, the polynucleotide encoding the α-GAL enzyme comprises a nucleotide sequence of SEQ ID NO: 12. In some embodiment, the polynucleotide encoding the α-GAL enzyme comprises a nucleotide sequence of SEQ ID NO: 13.

TABLE 2
nucleotide sequences of α-GAL and variants thereof

| Description | Sequence (5′ -3′ ) |
|---|---|
| WT | atgcagctgaggaacccagaactacatctgggctgcgcgcttgcgcttcgcttcctggccctcgtttcctgggacatccctg |
| | gggctagagcactggacaatggattggcaaggacgcctaccatgggctggctgcactgggagcgcttcatgtgcaacctt |
| | gactgccaggaagagccagattcctgcatcagtgagaagctcttcatggagatggcagagctcatggtctcagaaggctg |
| | gaaggatgcaggttatgagtacctctgcattgatgactgttggatggctccccaaagagattcagaaggcagacttcaggc |
| | agaccctcagcgctttcctcatgggattcgccagctagctaattatgttcacagcaaaggactgaagctagggatttatgca |
| | gatgttggaaataaaacctgcgcaggcttccctgggagttttggatactacgacattgatgcccagacctttgctgactggg |
| | gagtagatctgctaaaatttgatggttgttactgtgacagtttggaaaatttggcagatggttataagcacatgtccttggcc |
| | ctgaataggactggcagaagcattgtgtactcctgtgagtggcctctttatatgtggccctttcaaaagcccaattatacag |
| | aaatccgacagtactgcaatcactggcgaaattttgctgacattgatgattcctggaaaagtataaagagtatcttggact |
| | ggacatcttttaaccaggagagaattgttgatgttgctggaccagggggttggaatgacccagatatgttagtgattggca |
| | actttggcctcagctggaatcagcaagtaactcagatggccctctgggctatcatggctgctcctttattcatgtctaatgac |
| | ctccgacacatcagccctcaagccaaagctctccttcaggataaggacgtaattgccatcaatcaggaccccttgggcaag |
| | caagggtaccagcttagacagggagacaactttgaagtgtgggaacgacctctctcaggcttagcctgggctgtagctatg |
| | ataaaccggcaggagattggtggacctcgctcttataccatcgcagttgcttccctgggtaaaggagtggcctgtaatcctg |
| | cctgcttcatcacacagctcctccctgtgaaaaggaagctagggttctatgaatggacttcaaggttaagaagtcacataa |
| | atcccacaggcactgttttgcttcagctagaaaatacaatgcagatgtcattaaaagacttactttaa (SEQ ID NO: |
| | 8) |
| Codon- | TTGGACAATGGACTGGCAAGAACTCCTCCCATGGGCTGGCTGCACTGGGAAAGGTTTATGTG |
| optimized | TAATCTGGACTGTCAAGAGGAGCCAGATTCCTGCATCTCTGAGAAGCTCTTTGAAGAGATGG |
| CDS | CTGAGAGGATGGTGACAGATGGATGGAAGGATGCTGGATATGAGTACCTGTGCATTGATGA |
| (variant 1) | CTGCTGGATGGCCCCTCAGAGAGACAGTGAAGGCAGACTGCAAGCAGACCCCCAGAGGTTC |
| | CCCCATGGCATTAGGCAGCTGGCCAACCATGTCCACTCCAAGGGCCTGAAACTGGGCATCTA |
| | TGCTGATGTGGGCAATAAGACCTGTGCTGGTTTCCCTGGCTCCTTTGGATACTATGACATTGA |
| | TGCCCAGACCTTTGCTGACTGGGGTGTGGACCTGCTCAAGTTTGATGGCTGCTACTGTGACTC |
| | CCTGGAGAACCTGGCTGATGGTTACAAGCACATGTCTCTTGCTCTGAACAAGACTGGTAGAC |
| | CCATTGTTTACAGCTGTGAGTGGCCACTGTACATGTGGCCCTTCCAGAAGCCCAACTACACTG |
| | AGATCAGACAGTACTGCAATCATTGGAGGAATTTTGCAGATATTGATGATTCTTGGGCCTCC |
| | ATCAAGAGCATCCTGGACTGGACTTCCAGAAACCAAGAAAGAATTGTGGATGTTGCTGGCCC |
| | TGGAGGTTGGAATGACCCTGACATGCTGGTGATTGGCAACTTTGGGCTGTCCTGGGACCAGC |
| | AAGTGACTCAGATGGCCCTCTGGGCCATCATGGCTGGGCCCCTCTTCATGTCCAATGATCTGA |
| | GGGCCATTTCCCCCCAAGCCAAGGCCCTGCTTCAAGACAAAGATGTCATTGCTATCAATCAAG |
| | ATCCCCTGGGCAAGCAAGGCTACCAGCTCAGAAAAGGAGACAACTTTGAGGTGTGGGAGAG |
| | ACCTCTGTCTGGAGATGCCTGGGCTGTGGCCATCATCAACAGACAAGAGATTGGTGGCCCCA |
| | GAGGCTACACCATCCCTGTGGCTTCCCTGGGGAAGGGTGTTGCCTGCAACCCAGCTTGCTTC |
| | ATCACCCAGCTTCTGCCAGTGAAGAGGAAGCTGGGCTTCTATGAAGCTACCAGCAGACTGAG |
| | ATCCCACATCAACCCCACTGGCACAGTGCTGCTGCAGCTGGAAAACACCATGCAGACTTCTCT |
| | GAAGGACCTCCTGTGA (SEQ ID NO: 9) |
| Codon | CTGGATAATGGACTGGCAAGAACCCCAACCATGGGTTGGCTGCACTGGGAGAGGTTTATGT |
| Optimized | GTAATCTGGACTGTCAAGAGGAGCCAGACTCCTGCATCTCTGAGAAGCTCTTCATGGAGATG |
| CDS | GCTGAGAGGATGGTGAGTGAAGGCTGGAAGGATGCTGGTTATGAGTACCTGTGCATTGATG |
| (Variant 2) | ACTGCTGGATGGCCCCTCAGAGAGATTCAGAGGGCAGACTGCAAGCAGATCCCCAGAGGTT |
| | CCCCCATGGCATTAGGCAACTGGCCAACTATGTCCACTCCAAGGGCCTGAAGCTGGGCATCT |
| | ATGCTGATGTGGGCAACAAGACCTGTGCTGGCTTCCCTGGCTCCTTTGGCTACTATGATATTG |
| | ATGCCCAGACCTTTGCTGACTGGGGTGTGGACCTGCTCAAGTTTGATGGCTGCTACTGTGAC |
| | AGCCTGGAGAACCTGGCTGATGGTTACAAGCACATGTCTCTTGCTCTGAACAGAACTGGTAG |
| | AAGCATTGTTTACAGCTGTGAGTGGCCACTGTACATGTGGCCCTTCCAGAAGCCCAACTACA |
| | CTGAGATCAGACAGTACTGCAACCATTGGAGGAACTTTGCAGACATTGATGATTCCTGGGCC |
| | TCCATCAAGAGCATCCTGGACTGGACTTCCAGAAACCAAGAGAGAATTGTGGATGTTGCTGG |
| | ACCTGGAGGATGGAATGACCCTGACATGCTGGTGATTGGAAATTTTGGGCTGTCCTGGGACC |
| | AGCAAGTGACTCAGATGGCCAGCTGGGCCATCATGGCAGCCCCCCTGTTCATGAGCAATGAT |
| | CTCAGACACATTTCCCCCCAAGCCAAGGCCCTGCTTCAAGACAAAGATGTCATTGCTATTAAT |
| | CAAGATCCTTTGGGCAAGCAAGGCTACCAGCTGAGGAAGGGAGACAACTTTGAGGTGTGGG |
| | AAAGACCTCTGTCTGGAGATGCCTGGGCTGTGGCTATCATCAATAGACAAGAAATTGGTGGC |
| | CCCAGATCCTACACCATCCCTGTTGCCTCTCTGGGAAAAGGAGTGGCCTGCAATCCAGCTTGC |
| | TTCATCACCCAGCTCCTCCCAGTGAAGAGGCAGCTGGGTTTCTACAACTGGACAAGCAGATT |
| | GAAGTCCCACATCAACCCCACTGGCACAGTGCTGCTGCAGCTGGAAAACACCATGCAGATGT |
| | CCCTGAAGGACCTCCTGTGA (SEQ ID NO: 10) |
| Signal | atgcagctgaggaacccagaactacatctgggctgcgcgcttgcgcttcgcttcctggccctcgtttcctgggacatccctg |
| peptide | gggctagagca (SEQ ID NO: 11) |
| Variant 1 | atgcagctgaggaacccagaactacatctgggctgcgcgcttgcgcttcgcttcctggccctcgtttcctgggacatccctg |
| with signal | gggctagagcaTTGGACAATGGACTGGCAAGAACTCCTCCCATGGGCTGGCTGCACTGGGAAA |
| peptide | GGTTTATGTGTAATCTGGACTGTCAAGAGGAGCCAGATTCCTGCATCTCTGAGAAGCTCTTTG |
| | AAGAGATGGCTGAGAGGATGGTGACAGATGGATGGAAGGATGCTGGATATGAGTACCTGT |
| | GCATTGATGACTGCTGGATGGCCCCTCAGAGAGACAGTGAAGGCAGACTGCAAGCAGACCC |
| | CCAGAGGTTCCCCCATGGCATTAGGCAGCTGGCCAACCATGTCCACTCCAAGGGCCTGAAAC |
| | TGGGCATCTATGCTGATGTGGGCAATAAGACCTGTGCTGGTTTCCCTGGCTCCTTTGGATACT |
| | ATGACATTGATGCCCAGACCTTTGCTGACTGGGGTGTGGACCTGCTCAAGTTTGATGGCTGC |
| | TACTGTGACTCCCTGGAGAACCTGGCTGATGGTTACAAGCACATGTCTCTTGCTCTGAACAAG |
| | ACTGGTAGACCCATTGTTTACAGCTGTGAGTGGCCACTGTACATGTGGCCCTTCCAGAAGCC |
| | CAACTACACTGAGATCAGACAGTACTGCAATCATTGGAGGAATTTTGCAGATATTGATGATTC |
| | TTGGGCCTCCATCAAGAGCATCCTGGACTGGACTTCCAGAAACCAAGAAAGAATTGTGGATG |
| | TTGCTGGCCCTGGAGGTTGGAATGACCCTGACATGCTGGTGATTGGCAACTTTGGGCTGTCC |
| | TGGGACCAGCAAGTGACTCAGATGGCCCTCTGGGCCATCATGGCTGGGCCCCTCTTCATGTC |
| | CAATGATCTGAGGGCCATTTCCCCCCAAGCCAAGGCCCTGCTTCAAGACAAAGATGTCATTG |
| | CTATCAATCAAGATCCCCTGGGCAAGCAAGGCTACCAGCTCAGAAAAGGAGACAACTTTGAG |
| | GTGTGGGAGAGACCTCTGTCTGGAGATGCCTGGGCTGTGGCCATCATCAACAGACAAGAGA |
| | TTGGTGGCCCCAGAGGCTACACCATCCCTGTGGCTTCCCTGGGGAAGGGTGTTGCCTGCAAC |
| | CCAGCTTGCTTCATCACCCAGCTTCTGCCAGTGAAGAGGAAGCTGGGCTTCTATGAAGCTAC |
| | CAGCAGACTGAGATCCCACATCAACCCCACTGGCACAGTGCTGCTGCAGCTGGAAAACACCA |
| | TGCAGACTTCTCTGAAGGACCTCCTGTGA (SEQ ID NO: 12) |
| Variant 2 | Atgcagctgaggaacccagaactacatctgggctgcgcgcttgcgcttcgcttcctggccctcgtttcctgggacatccctg |
| with signal | gggctagagcaCTGGATAATGGACTGGCAAGAACCCCAACCATGGGTTGGCTGCACTGGGAGA |
| peptide | GGTTTATGTGTAATCTGGACTGTCAAGAGGAGCCAGACTCCTGCATCTCTGAGAAGCTCTTC |
| | ATGGAGATGGCTGAGAGGATGGTGAGTGAAGGCTGGAAGGATGCTGGTTATGAGTACCTG |
| | TGCATTGATGACTGCTGGATGGCCCCTCAGAGAGATTCAGAGGGCAGACTGCAAGCAGATC |
| | CCCAGAGGTTCCCCCATGGCATTAGGCAACTGGCCAACTATGTCCACTCCAAGGGCCTGAAG |
| | CTGGGCATCTATGCTGATGTGGGCAACAAGACCTGTGCTGGCTTCCCTGGCTCCTTTGGCTAC |
| | TATGATATTGATGCCCAGACCTTTGCTGACTGGGGTGTGGACCTGCTCAAGTTTGATGGCTG |
| | CTACTGTGACAGCCTGGAGAACCTGGCTGATGGTTACAAGCACATGTCTCTTGCTCTGAACA |
| | GAACTGGTAGAAGCATTGTTTACAGCTGTGAGTGGCCACTGTACATGTGGCCCTTCCAGAAG |
| | CCCAACTACACTGAGATCAGACAGTACTGCAACCATTGGAGGAACTTTGCAGACATTGATGA |
| | TTCCTGGGCCTCCATCAAGAGCATCCTGGACTGGACTTCCAGAAACCAAGAGAGAATTGTGG |
| | ATGTTGCTGGACCTGGAGGATGGAATGACCCTGACATGCTGGTGATTGGAAATTTTGGGCTG |
| | TCCTGGGACCAGCAAGTGACTCAGATGGCCAGCTGGGCCATCATGGCAGCCCCCCTGTTCAT |
| | GAGCAATGATCTCAGACACATTTCCCCCCAAGCCAAGGCCCTGCTTCAAGACAAAGATGTCA |
| | TTGCTATTAATCAAGATCCTTTGGGCAAGCAAGGCTACCAGCTGAGGAAGGGAGACAACTTT |
| | GAGGTGTGGGAAAGACCTCTGTCTGGAGATGCCTGGGCTGTGGCTATCATCAATAGACAAG |
| | AAATTGGTGGCCCCAGATCCTACACCATCCCTGTTGCCTCTCTGGGAAAAGGAGTGGCCTGC |
| | AATCCAGCTTGCTTCATCACCCAGCTCCTCCCAGTGAAGAGGCAGCTGGGTTTCTACAACTGG |
| | ACAAGCAGATTGAAGTCCCACATCAACCCCACTGGCACAGTGCTGCTGCAGCTGGAAAACAC |
| | CATGCAGATGTCCCTGAAGGACCTCCTGTGA (SEQ ID NO: 13) |

In some embodiments, the polynucleotide is codon optimized. In some embodiments, the polynucleotide of the GLA gene therapy vector that encodes α-GAL enzyme or a variant thereof, comprises a nucleic acid sequence comprising at least 70%, 75%, 80%, 85%, 90%, 95%, 96%, 97%, 98%, 99%, or more than 99% identity to any one of SEQ ID NOs: 8-10 and 12-13.

In some embodiments, the polynucleotide encoding α-GAL or variant thereof comprises a nucleic acid sequence having at least 70%, 75%, 80%, 85%, 90%, 95%, 96%, 97%, 98%, 99%, or more than 99% identity to SEQ ID NO: 8. In some embodiments, the polynucleotide encoding α-GAL or variant thereof comprises a nucleic acid sequence having at least 70%, 75%, 80%, 85%, 90%, 95%, 96%, 97%, 98%, 99%, or more than 99% identity to SEQ ID NO:9. In some embodiments, the polynucleotide encoding α-GAL or variant thereof comprises a nucleic acid sequence having at least 70%, 75%, 80%, 85%, 90%, 95%, 96%, 97%, 98%, 99%, or more than 99% identity to SEQ ID NO: 10. In some embodiments, the polynucleotide encoding α-GAL or variant thereof comprises a nucleic acid sequence having at least 70%, 75%, 80%, 85%, 90%, 95%, 96%, 97%, 98%, 99%, or more than 99% identity to SEQ ID NO: 12. In some embodiments, the polynucleotide encoding α-GAL or variant thereof comprises a nucleic acid sequence having at least 70%, 75%, 80%, 85%, 90%, 95%, 96%, 97%, 98%, 99%, or more than 99% identity to SEQ ID NO: 13.

In other embodiments, the polynucleotide encoding α-GAL or variant thereof may comprises a nucleic acid sequence disclosed in the applicant's previous PCT application NO: PCT/US22/17998 (e.g., sequences in Table 1 of PCT/US22/17998); the contents of which are incorporated herein by reference herein in their entirety.

### Recombinant AAV (rAAV) vectors

Transgenes delivered by a vector can be introduced into a cell of interest using a variety of methods. For example, either viral or non-viral vectors can be used for the delivery of a transgene of interest. Both viral and non-viral methods of vector delivery are contemplated by the methods provided herein. Accordingly, in some embodiments, the vector described herein is delivered in a viral vector. In some embodiments, the vector described herein is delivered in a non-viral vector. In some embodiments, the vectors can be introduced as naked nucleic acids, as nucleic acid complexed with an agent such as a liposome or a poloxamer. Various viral vectors are known in the art for delivery of transgenes, and include for example either integrating or non-integrating vectors. In some embodiments, the viral vector is a non-integrating viral vector. Non-integrating viral vectors include, for example non-integrating lentivirus vectors or AAV vectors. In other embodiments, the GLA transgene of the present invention can be delivered by viruses (e.g., adenovirus, adeno-associated virus (AAV), herpesvirus, retrovirus, lentivirus and integrase defective lentivirus (IDLV). The vector may have additional sequences, such as, for example, replication origins, promotor and one or more genes.

As non-limiting examples, the GLA transgene described herein is introduced using a viral vector; the viral vector is adeno-associated virus (AAV) derived vector. In accordance with the present disclosure, a recombinant adeno-associated viral (rAAV) vector is used for the GLA transgene.

Generally, a rAAV vector comprises a capsid and a viral genome which is modified to include a transgene, e.g., a transgene for expressing an α-GAL enzyme (i.e., a polynucleotide encoding an α-GAL enzyme or a variant thereof. AAV vectors described herein may comprise or be derived from any natural or recombinant AAV serotype. AAV serotypes may differ in characteristics such as, but not limited to, packaging, tropism, transduction and immunogenic profiles. While not wishing to be bound by theory, the AAV capsid protein is often considered to be the driver of AAV particle tropism to a particular tissue.

In many gene therapy applications, it is desirable that the gene therapy vector be delivered with a specificity to a particular tissue type. Prior gene therapy approaches for the treatment of Fabry disease have met with limited success because of reduced tissue tropism for the gene therapy vectors that have previously been used. Unlike the previously used vector designs, the vector design provided here has a wide tissue and cell type distribution once administered to a subject in need thereof. The rAAV vectors described herein have broad tissue distribution and include, for example heart, liver, kidney, gastrointestinal tract and nervous tissues.

Various kinds of AAV capsids with broad tissue tropism (the terms “broad tissue tropism” “wide-tropism” are used interchangeably herein) can be used in the rAAV vector described herein. Various kinds of capsids and associated tropism are described in Curr Opin Vir. 2016 Dec. 21:75-80, the contents of which are incorporated herein by reference. By “broad tissue tropism” it is meant that the capsid is able to enable gene transfer to two or more than 2, 3, 4, 5, 6, 7, 8 or more tissue types. For example, in some embodiments, a capsid having broad tissue tropism enable gene transfer to one or more of the following tissues: liver, kidney, heart, gastrointestinal tract, and/or peripheral neurons of the subject.

For example, in some embodiments, the AAV capsid is a wide-tropism AAV capsid selected from an AAV1 capsid, AAV2 capsid, AAV3 capsid, AAV4 capsid, AAV5 capsid, AAV6 capsid, AAV7 capsid, AAV8 capsid, AAV9 capsid, AAV11, 12,13, AAVhu.37, AAVrh.8, AAVrh.10, and AAVrh.39, AAV-DJ, or AAV-DJ/8.

Accordingly, in some embodiments, the AAV capsid with wide-tropism comprises an AAV1 capsid. In some embodiments, the AAV capsid with wide-tropism is comprises an AAV2 capsid. In some embodiments, the AAV capsid with wide-tropism comprises an AAV3 capsid. In some embodiments, the AAV capsid with wide-tropism comprises an AAV4 capsid. In some embodiments, the AAV capsid with wide-tropism comprises an AAV5 sequence. In some embodiments, the AAV capsid with wide-tropism comprises an AAV6 capsid. In some embodiments, the AAV capsid with wide-tropism comprises an AAV7 capsid. In some embodiments, the AAV capsid with wide-tropism comprises an AAV8 capsid. In some embodiments, the AAV capsid with wide-tropism comprises an AAV9 capsid.

While not wishing to be bound by theory, it is understood that an AAV capsid sequence comprises a VP1 region. In some embodiments, a parent AAV capsid sequence comprises a VP1, VP2 and/or VP3 region, or any combination thereof. A parent VP1 sequence may be considered synonymous with a parent AAV capsid sequence.

In some embodiments, the rAAV capsid sequence may comprise an amino acid sequence with 75%, 80%, 90%, 91%, 92%, 93%, 94%, 95%, 96%, 97%, 98%, 99%, or 100% identity to any of the those described above.

Recombinant or engineered AAV vectors may comprise a capsid engineered with enhanced tropism for targeting broad tissues, or for targeting nervous tissues (e.g., DRG). Capsid engineering methods have been used to try to identify capsids with enhanced transduction of broad tissues (e.g., kidney, liver, lung, heart, brain, spinal cord, DRG). A variety of methods have been used, including mutational methods, DNA barcoding, directed evolution, random peptide insertions, and capsid shuffling and/or chimeras.

In one preferred embodiment, the rAAV vector described herein comprises an AAV9 capsid sequence. In some examples, the rAAV vector described herein comprises a capsid sequence having 75%, 80%, 90%, 91%, 92%, 93%, 94%, 95%, 96%, 97%, 98%, 99%, or 100% identity to an AAV9 capsid sequence.

The AAV vector comprises one or more Inverted terminal repeat (ITR) sequences. Inverted terminal repeats (ITRs) traditionally cap the viral genome at both the 5′ and the 3′ end, providing origins of replication for the viral genome. While not wishing to be bound by theory, an AAV viral vector typically comprises two ITR sequences at the 5′ and 3′ ends of the ssDNA which form an energetically stable double stranded region. In some embodiments, AAV vectors are recombinant AAV viral vectors which are replication defective and lacking sequences encoding functional Rep and Cap proteins within their viral genome. These defective AAV vectors may lack most or all parental coding sequences and essentially carry only one or two AAV ITR sequences and the GLA transgene for delivery to a cell, a tissue, an organ, or an organism.

In some embodiments, the rAAV vector described herein comprise at least one ITR and a GLA transgene. In some embodiments, the rAAV vector has two ITRs. These two ITRs flank the transgene region at the 5′ and 3′ ends. The ITRs function as origins of replication comprising recognition sites for replication. ITRs comprise sequence regions which can be complementary and symmetrically arranged. ITRs incorporated into viral genomes as described herein may be comprised of naturally occurring polynucleotide sequences or recombinantly derived polynucleotide sequences. The ITRs may be derived from the same serotype as the AAV capsid, selected from any of the known AAV serotypes, or a derivative thereof. The ITR may be of a different serotype than the capsid.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In some embodiments, the ITRs are 140-142 nucleotides in length. Non-limiting examples of ITR length are 102, 105, 130, 140, 141, 142, 145 nucleotides in length. ITRs encompassed by the present disclosure include those with at least 70% identity, at least 75% identity, at least 80% identity, at least 85% identity, at least 90% identity, at least 95% identity, at least 98% identity, or at least 99% identity to a known AAV serotype ITR sequence.

In some embodiments, the rAAV vector comprises at least one element to enhance the transgene specificity and expression (See e.g., Powell et al. Viral Expression Cassette Elements to Enhance Transgene Target Specificity and Expression in Gene Therapy, 2015). Non-limiting examples of elements to enhance transgene specificity and expression include promoters, endogenous miRNAs, post-transcriptional regulatory elements (PREs), polyadenylation (PolyA) signal sequences and upstream enhancers (USEs), CMV enhancers and introns.

A person skilled in the art may recognize that expression of a transgene in a target cell may require a specific promoter, including but not limited to, a promoter that is species specific, inducible, tissue-specific, or cell cycle-specific (Parr et al., Nat. Med. 1997, 3:1145-1149). In some embodiments, the promoter is deemed to be efficient when it drives expression of the transgene (e.g., the GLA transgene) of the rAAV vector. In some embodiments, the promoter is a promoter deemed to be efficient when it drives expression in a cell being targeted. In some embodiments, the promoter is a promoter having a tropism for a cell being targeted, such as a neuron.

As a non-limiting example, the promoter is a selected for sustained expression of a transgene in tissues and/or cells of the central or peripheral nervous system.

Promoters may be naturally occurring or non-naturally occurring. Non-limiting examples of promoters include those derived from viruses, plants, mammals, or humans. In some embodiments, the promoters may be those derived from human cells or systems. In some embodiments, the promoter may be truncated or mutated.

Promoters which drive or promote expression in most tissues include, but are not limited to the human elongation factor 1Į-subunit (EF1Į) promoter, the cytomegalovirus (CMV) immediate-early enhancer and/or promoter, the chicken ü-actin (CBA) promoter and its derivative CAG, ü glucuronidase (GUSB) promoter, or ubiquitin C (UBC) promoter. In some embodiments, the viral vector comprises a ubiquitous promoter. Non-limiting examples of ubiquitous promoters include EF-1Į, PGK, UBC, GUSB (hGBp), and UCOE (promoter of HNRPA2B1-CBX3),

In some embodiments, the promoter sequence is a ubiquitous promoter sequence. In some embodiments, the promoter is a mammalian ubiquitous promoter promoting expression of a coding sequence (e.g., GLA transgene) in mammalian cells. In some embodiments, the rAAV vector expressing a GLA transgene with a ubiquitous promoter achieves broad distribution of encoded α-GAL to multiple target tissues such as kidney, liver, lung, heart and the nervous system in a mammal, thereby resulting in broader exposure of α-GAL and better treatment of Fabry disease and the associated symptoms. As non-limiting examples, the ubiquitous promoter as used in this disclosure can be selected from one or more of EF-1α promoter, UBC promoter, LSE beta-glucuronidase (GUSB) promoter, ubiquitous chromatin opening element (UCOE) promoter, GAPDH promoter, chicken β actin (CBA) promoter, PGK promoter, CMV promoter, and mini EF1 promoter. In some embodiments, the ubiquitous promoter can be engineered from one of more known ubiquitous promoters. In some embodiments, the ubiquitous promoter comprises a cyto-megalo-virus (CMV) enhancer, chicken beta actin promoter, and a rabbit beta globin intron.

In some embodiments, cell-type specific promoters may be used to increase exposure of a GLA transgene to excitatory neurons (e.g., glutamatergic), inhibitory neurons (e.g., GABA-ergic), neurons of the sympathetic or parasympathetic nervous system, sensory neurons, neurons of the dorsal root ganglia, dorsal root nerves, motor neurons, or supportive cells of the nervous systems such as microglia, astrocytes, oligodendrocytes, and/or Schwann cells.

In some embodiments, the promoter may be a combination of two or more components of the same or different starting or parental promoters.

In some embodiments, the rAAV vector components can be selected and/or engineered to further tailor the specificity and efficiency of expression of the GLA transgene in the nervous system (e.g., DRG).

In some embodiments, the rAAV vector optionally comprises a woodchuck hepatitis virus posttranscriptional regulatory element (WPRE) (e.g., having mut6delATG mutation). In some examples, the WPRE element locates between the nucleic acid sequence encoding an α-GAL enzyme or variant thereof and a polyA sequence.

In some embodiments, in order to further increase the expression of the GLA transgene in the nervous system for alleviating neuropathy in Fabry disease (e.g., DRG and other cells of the CNS and PNS) while using a capsid sequence with a broad tropism and a ubiquitous promoter, the rAAV vector may optionally further include a targeting peptide of the nervous system. In some embodiments, the targeting peptide may direct an AAV particle to a cell or tissue of the PNS. The cell or tissue of the PNS may be, but is not limited to, a dorsal root ganglion (DRG). A targeting peptide may vary in length. In some embodiments, the targeting peptide is 3-20 amino acids in length. As non-limiting examples, the targeting peptide may be 3, 4, 5, 6, 7, 8, 9, 10, 11, 12, 13, 14, 15, 16, 17, 18, 19, 20 or 3-5, 3-8, 3-10, 3-12, 3-15, 3-18, 3-20, 5-10, 5-15, 5-20, 10-12, 10-15, 10-20, 12-20, or 15-20 amino acids in length.

In certain embodiments, the rAAV vector further comprises a posttranscriptional regulatory element (PRE). In some embodiments, the vector comprises woodchuck hepatitis virus post-transcriptional control element (WPRE). Various optimized or variant forms of WPRE are known in the art, and include WPRE3, WPREmut6delATG among others. Other variant WPRE forms include, for example, WPRE2, WPRE_wt (GenBank accession no. J04514); WPRE_wt (GenBank accession no. J02442) and WPREmut6. The WPRE element can comprises a wild-type sequence or a modified WPRE element sequence. Various mutated versions of WPRE are known, and include for example, mut6delATG (SEQ ID NO: 17). In some embodiments, the vector comprises mut6delATG (SEQ ID NO: 17).

In some embodiments, the present disclosure encompasses a gene therapy vector comprising a GLA gene sequence that is modified. Such modification may be made to improve expression characteristics. Such modifications can include, but are not limited to, insertion of a translation start site (e.g. methionine), addition of a Kozak sequence (gccacca), insertion of a signal peptide, and/or codon optimization. Accordingly, in some embodiments, the GLA gene is modified to include insertion of a translation start site. In some embodiments, the GLA gene is modified to include the addition of a Kozak sequence. In some embodiments, the GLA gene is modified to comprise a signal peptide. In some embodiments, the GLA gene is codon optimized. In other embodiments, the GLA gene is engineered. In yet other embodiments, the GLA gene is codon optimized and engineered.

In some embodiments, the vector described herein comprises one or more polyA sequences. In some embodiments, the polyA is selected from human growth hormone polyA (hGHpA), synthetic polyA (SPA), Simian virus 40 late poly A (SV40pA) and a bovine growth hormone (BGH) poly A.

In some embodiments, the vector comprises an ID tag, e.g., a stuffer sequence. The purpose of the ID tag includes for example the ability for an artisan to identify the vector. One example of DNA tag sequence is listed in Table 3 (SEQ ID NO: 20).

In some embodiments, the AAV vector is modified at one or more regions, such as the AAV capsid. In some embodiments, the rAAV vector is a rAAV9 vector.

Exemplary sequences for the rAAV vector are shown in Table 2 below. In some embodiments, the rAAV vector comprises a rAAV vector element comprising a nucleotide sequence having at least 60%, 65%, 70%, 75%, 80%, 85%, 90%, 95%, 96%, 97%, 98%, or 99% identity with a vector element sequence shown in Table 3 below. In some embodiments, the rAAV vector comprises a vector element nucleotide sequence identical to a vector element nucleotide sequence shown in Table 3 below.

TABLE 3
Exemplary Vector Component Sequences
5′ITR
Ctgcgcgctcgctcgctcactgaggccgcccgggcaaagcccgggcgtcgggcgacctttggtcgcccggcctcagtgagcgag
cgagcgcgcagagagggagtggccaactccatcactaggggttcct (SEQ ID NO: 14)
Chicken-Beta (CB) promoter
gatcttcaatattggccattagccatattattcattggttatatagcataaatcaatattggctattggccattgcatacgttgtatct
atatcataatatgtacatttatattggctcatgtccaatatgaccgccatgttggcattgattattgactagttattaatagtaatca
attacggggtcattagttcatagcccatatatggagttccgcgttacataacttacggtaaatggcccgcctggctgaccgcccaa
cgacccccgcccattgacgtcaataatgacgtatgttcccatagtaacgccaatagggactttccattgacgtcaatgggtggag
tatttacggtaaactgcccacttggcagtacatcaagtgtatcatatgccaagtccgccccctattgacgtcaatgacggtaaatg
gcccgcctggcattatgcccagtacatgaccttacgggactttcctacttggcagtacatctacgtattagtcatcgctattaccat
ggtcgaggtgagccccacgttctgcttcactctccccatctcccccccctccccacccccaattttgtatttatttattttttaattatt
ttgtgcagcgatgggggcggggggggggggggggcgcgcgccaggcggggggggcggggcgaggggggggcggggcga
ggcggagaggtgcggcggcagccaatcagagcggcgcgctccgaaagtttccttttatggcgaggcggcggcggcggcggccc
tataaaaagcgaagcgcgcggggggggagtcgctgcgacgctgccttcgccccgtgccccgctccgccgccgcctcgcgccg
cccgccccggctctgactgaccgcgttactcccacaggtgagcggggggacggcccttctcctccgggctgtaattagcgcttg
gtttaatgacggcttgtttcttttctgtggctgcgtgaaagccttgaggggctccgggagggccctttgtgcgggggggagcggct
cggggggtgcgtgcgtgtgtgtgtgcgtggggagcgccgcgtgcggcccgcgctgcccggcggctgtgagcgctgcgggcgcgg
cgcggggctttgtgcgctccgcagtgtgcgcgaggggagcgcggccgggggcggtgccccgcggtgcggggggggctgcgagg
ggaacaaaggctgcgtgcggggtgtgtgcgtgggggggtgagcagggggtgtgggcgcggcggtcgggctgtaacccccccct
gcacccccctccccgagttgctgagcacggcccggcttcgggtgcggggctccgtacggggcgtggcgcggggctcgccgtgcc
gggcggggggtggcggcaggtgggggtgccgggcggggcggggccgcctcgggccggggagggctcgggggaggggcgcgg
cggcccccggagcgccggcggctgtcgaggcgcggcgagccgcagccattgccttttatggtaatcgtgcgagagggcgcagg
gacttcctttgtcccaaatctgtgcggagccgaaatctgggaggcgccgccgcaccccctctagcgggcgcggggcgaagcggt
gcggcgccggcaggaaggaaatgggcggggagggccttcgtgcgtcgccgcgccgccgtccccttctccctctccagcctcggg
gctgtccgcggggggacggctgccttcgggggggacggggcagggcggggttcggcttctggcgtgtgaccggcggctctaga
gcctctgctaaccatgttcatgccttcttctttttcctacagctcctgggcaacgtgctggttattgtgctgtctcatcattttggcaa
agaattcgatatca (SEQ ID NO: 15)
WT-hGLA1
atgcagctgaggaacccagaactacatctgggctgcgcgcttgcgcttcgcttcctggccctcgtttcctgggacatccctggggc
tagagcactggacaatggattggcaaggacgcctaccatgggctggctgcactgggagcgcttcatgtgcaaccttgactgcca
ggaagagccagattcctgcatcagtgagaagctcttcatggagatggcagagctcatggtctcagaaggctggaaggatgcag
gttatgagtacctctgcattgatgactgttggatggctccccaaagagattcagaaggcagacttcaggcagaccctcagcgcttt
cctcatgggattcgccagctagctaattatgttcacagcaaaggactgaagctagggatttatgcagatgttggaaataaaacct
gcgcaggcttccctgggagttttggatactacgacattgatgcccagacctttgctgactggggagtagatctgctaaaatttgat
ggttgttactgtgacagtttggaaaatttggcagatggttataagcacatgtccttggccctgaataggactggcagaagcattgt
gtactcctgtgagtggcctctttatatgtggccctttcaaaagcccaattatacagaaatccgacagtactgcaatcactggcgaa
attttgctgacattgatgattcctggaaaagtataaagagtatcttggactggacatcttttaaccaggagagaattgttgatgttg
ctggaccagggggttggaatgacccagatatgttagtgattggcaactttggcctcagctggaatcagcaagtaactcagatggc
cctctgggctatcatggctgctcctttattcatgtctaatgacctccgacacatcagccctcaagccaaagctctccttcaggataa
ggacgtaattgccatcaatcaggaccccttgggcaagcaagggtaccagcttagacagggagacaactttgaagtgtgggaac
gacctctctcaggcttagcctgggctgtagctatgataaaccggcaggagattggtggacctcgctcttataccatcgcagttgct
tccctgggtaaaggagtggcctgtaatcctgcctgcttcatcacacagctcctccctgtgaaaaggaagctagggttctatgaatg
gacttcaaggttaagaagtcacataaatcccacaggcactgttttgcttcagctagaaaatacaatgcagatgtcattaaaagac
ttactttaa (SEQ ID NO: 8)
WPREmut6delATG
Aatcaacctctggattacaaaatttgtgaaagattgactggtattcttaactttgttgctccttttacgctttgtggatacgctgcttt
attgcctttgtatcttgctattgcttcccgtttggctttcattttctcctccttgtataaatcctggttgctgtctctttttgaggagttgt
ggcccgttgtcaggcaacgtggcgtggtgtgcactgtgtttgctgacgcaacccccactggttggggcattgccaccacctgtcag
ctcctttccgggactttcgctttccccctccctattgccacggcggaactcatcgccgcctgccttgcccgctgctggacaggggct
cggctgttgggcactgacaattccgtggtgttgtcggggaaatcatcgtcctttccttggctgctcgcctgtgttgccacctggattc
tgcgcgggacgtccttctgctacgtcccttcggccctcaatccagcggaccttccttcccgcggcctgctgccggctctgcggcctc
ttccgcgtcttcgccttcgccctcagacgagtcggatctccctttgggccgcctccccgcatc (SEQ ID NO: 16)
bovine growth hormone polyadenylation signal (bGHpolyA)
Cctagagctcgctgatcagcctcgactgtgccttctagttgccagccatctgttgtttgcccctcccccgtgccttccttgaccctgg
aaggtgccactcccactgtcctttcctaataaaatgaggaaattgcatcgcattgtctgagtaggtgtcattctattctggggggtg
gggtggggcaggacagcaagggggaggattgggaagacaatagcaggcatgctggggaa (SEQ ID NO: 17)
3′ ITR
Aggaacccctagtgatggagttggccactccctctctgcgcgctcgctcgctcactgaggccgggcgaccaaaggtcgcccgac
gcccgggctttgcccgggcggcctcagtgagcgagcgagcgcgcagaga (SEQ ID NO: 18)

In some embodiments, the present disclosure encompasses a rAAV vector packaged in an AAV capsid having broad tissue tropism, where the rAAV vector comprises: a) 5′ inverted terminal repeat (ITR); b) a ubiquitous promoter; c).a polynucleotide encoding α-GAL enzyme or variant thereof; d) a poly A; and e) a 3′ ITR sequence.

In some embodiments, the rAAV vector further comprises a posttranscriptional regulatory element (PRE). In some embodiments, a rAAV vector comprises a woodchuck hepatitis virus posttranscriptional regulatory element (WPRE) (e.g., having mut6delATG mutation) between a nucleotide sequence encoding an α-GAL enzyme and a polyA sequence. As a non-limiting example, the rAAV vector comprises: a) a 5′ inverted terminal repeat (ITR); b) a ubiquitous promoter; c).a polynucleotide encoding α-GAL enzyme or variant thereof; d) a woodchuck hepatitis virus posttranscriptional regulatory element (WPRE); e) a poly A; and f) a 3′ ITR sequence.

In other embodiments, a rAAV vector that is able to achieve broad α-GAL enzyme expression upon administration to a subject in need comprises: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding an α-GAL enzyme or variant thereof; (d) a bovine growth hormone (BGH) poly A; and (e) a 3′ ITR.

In some embodiments, a rAAV vector that is able to achieve broad α-GAL enzyme expression upon administration to a subject in need comprises: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding an α-GAL enzyme or a variant thereof; (d) a woodchuck hepatitis virus posttranscriptional regulatory element (WPRE) having mut6delATG mutation; (e) a bovine growth hormone (BGH) poly A; and (f) a 3′ ITR.

In some embodiments, a rAAV vector that is able to achieve broad α-GAL enzyme expression upon administration to a subject in need is packaged in an AAV9 capsid, the rAAV vector comprises: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding an α-GAL enzyme or variant thereof; (d) a bovine growth hormone (BGH) poly A; and (e) a 3′ ITR sequence.

In some embodiments, a rAAV vector that is able to achieve broad α-GAL enzyme expression upon administration to a subject in need is packaged in an AAV9 capsid, the rAAV vector comprises: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding an α-GAL enzyme or variant thereof; (d) a woodchuck hepatitis virus posttranscriptional regulatory element (WPRE) having mut6delATG mutation; (e) a bovine growth hormone (BGH) poly A; and (f) a 3′ ITR sequence.

In some embodiments, the disclosure provides an expression cassette comprising a polynucleotide sequence comprising: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding α-GAL enzyme; (d) optionally a woodchuck hepatitis virus posttranscriptional regulatory element (WPRE) comprising the mut6delATG mutation; (e) a bovine growth hormone (BGH) poly A; and (f) a 3′ ITR. In some embodiments, the elements in the expression cassette above are present in 5′ to 3′ order. In various embodiments, one or more of (a) to (f) are operably linked in 5′ to 3′ order.

In one example, the present disclosure provides a rAAV vector packaged in an AAV capsid, said rAAV vector comprises (a) a 5′ inverted terminal repeat (ITR) comprising SEQ ID NO: 14; (b) a promoter comprising SEQ ID NO; 15; (c) a nucleotide sequence encoding α-GAL enzyme comprising an amino acid sequence of any one of SEQ ID Nos: 1-3 and 5-7; (d) a bovine growth hormone (BGH) poly A comprising SEQ ID NO: 17; and (e) a 3′ ITR sequence comprising SEQ ID NO: 18.

In another example, the present disclosure provides a rAAV vector packaged in an AAV capsid, said rAAV vector comprises (a) a 5′ inverted terminal repeat (ITR) comprising SEQ ID NO: 14; (b) a promoter comprising SEQ ID NO; 15; (c) a nucleotide sequence encoding α-GAL enzyme comprising an amino acid sequence of any one of SEQ ID Nos: 1-3 and 5-7; (d) a woodchuck hepatitis virus posttranscriptional regulatory element (WPRE) comprising SEQ ID NO: 17; (e) a bovine growth hormone (BGH) poly A comprising SEQ ID NO: 18; and (f) a 3′ ITR sequence comprising SEQ ID NO: 18.

As non-limiting examples, the present disclosure provides a rAAV vector comprising (a) a 5′ inverted terminal repeat (ITR) comprising SEQ ID NO: 14; (b) a promoter comprising SEQ ID NO; 15; (c) a nucleotide sequence that encodes α-GAL enzyme or variant thereof comprising any one of SEQ ID Nos: 8-10 and 12-13; (d) a bovine growth hormone (BGH) poly A comprising SEQ ID NO: 17; and (e) a 3′ ITR sequence comprising SEQ ID NO: 18. In some embodiments, the present disclosure provides a rAAV vector comprising (a) a 5′ inverted terminal repeat (ITR) comprising SEQ ID NO: 14; (b) a promoter comprising SEQ ID NO; 15; (c) a nucleotide sequence that encodes □-GAL enzyme or variant thereof comprising any one of SEQ ID Nos: 8-10 and 12-13; d) a woodchuck hepatitis virus posttranscriptional regulatory element (WPRE) comprising SEQ ID NO: 16; (e) a bovine growth hormone (BGH) poly A comprising SEQ ID NO: 17; and (f) a 3′ ITR sequence comprising SEQ ID NO: 18.

### Production of rAAV Vectors

In various embodiments, a rAAV vector described herein for delivering a transgene (e.g., a gene encoding an alpha-galactosidase (α-GAL) protein) can be packaged using techniques known in the art and as described herein. For example, in some embodiments, rAAV packaging makes use of packaging cells to form virus particles that are capable of infecting a host cell, such as HEK293, HeLa, HEK293T, Sf9 cells or A549 cells.

Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette encoding the protein to be expressed. In this case the protein to be expressed is α-GAL enzyme or variant thereof. The missing viral functions can be supplied in trans by the packaging cell line. For example, AAV vectors used in gene therapy typically only possess inverted terminal repeat (ITR) sequences from the AAV genome which are required for packaging and integration into the host genome.

Methods for generating and isolating AAV viral vectors suitable for delivery to a subject are known in the art. See, e.g., U.S. Pat. Nos. 7,790,449, 7,282,199, and 7,588,772; PCT publications WO 2003/042397, WO 2005/033321 and WO 2006/110689. In a one system, a packaging cell line is transiently transfected with a construct that encodes the transgene flanked by ITRs and a construct(s) that encodes the AAV rep and cap proteins. In a second system, a packaging cell line that stably supplies rep and cap is transiently transfected with a construct encoding the transgene flanked by ITRs. In each of these systems, AAV virions are produced in response to infection with helper adenovirus or herpesvirus (e.g., adenovirus E1, E2a, VA, and E4 or herpesvirus UL5, UL8, UL52, and UL29, and herpesvirus polymerase), which help the separation of the rAAV vectors from contaminating viruses.

Other systems that do not require infection with helper virus to recover the AAV can be used. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level.

In some embodiments, the expression cassette flanked by ITRs and rep/cap genes are introduced into a packaging cell by infection with baculovirus-based vectors (e.g., Zhang et al, Adenovirus-adeno-associated virus hybrid for large-scale recombinant adeno-associated virus production; Human Gene Therapy, 2009; 20:922-929, the contents of which is incorporated herein by reference in its entirety). Other AAV production systems and methods are also described, for example, in U.S. Pat. Nos: 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065; the contents of each of which are incorporated by reference herein in their entirety.

Methods and technologies for generating transgene expression cassettes, rAAV vectors and helper plasmids and constructs, such as genetic engineering, recombinant engineering, and synthetic techniques, are well-known in the art (See, e.g., Green and Sambrook et al, Molecular Cloning: A Laboratory Manual, Cold Spring Harbor Press, Cold Spring Harbor, NY (2012)) Similarly, methods of generating rAAV virions are well known and the selection of a suitable method is not a limitation on the present invention.

Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

In certain embodiments, the rAAV expression cassette, the vector (such as rAAV vector), the virus (such as rAAV), the production plasmid comprises AAV inverted terminal repeat sequences, a codon optimized nucleic acid sequence that encodes an α-GAL polypeptide, and expression control sequences that direct expression of the encoded proteins are present in a host cell. In other embodiments, the rAAV expression cassette, the virus, the vector (such as rAAV vector), the production plasmid further comprise one or more of an intron, a Kozak sequence, a polyA, posttranscriptional regulatory elements and others. In one embodiment, the post-transcriptional regulatory element is Woodchuck Hepatitis Virus (WHP) Posttranscriptional Regulatory Element (WPRE). In various embodiments, the nucleic acid sequence comprises a signal peptide upstream of the transgene that encodes an α-GAL polypeptide. In some embodiments, a signal peptide is at the N-terminus of an α-GAL polypeptide. In some embodiments, a signal peptide is at the C-terminus of an α-GAL polypeptide.

In some embodiments, the helper plasmids include a first helper plasmid comprising a rep gene and a cap gene, and a second helper plasmid comprising one or more of the following helper genes: E1a gene, E1b gene, E4 gene, E2a gene, and VA gene. For clarity, helper genes are genes that encode helper proteins E1a, E1b, E4, E2a, and VA. In some embodiments, the cap gene is modified such that one or more of the proteins VP1, VP2 and VP3 do not get expressed. In some embodiments, the cap gene is modified such that VP2 does not get expressed. Methods for making such modifications are known in the art (Lux et al. (2005), J Virology, 79:11776-87). Helper plasmids, and methods of making such plasmids, are generally known in the art and generally commercially available (see, e.g., pDF6, pRep, pDM, pDG, pDPIrs, pDP2rs, pDP3rs, pDP4rs, pDP5rs, pDP6rs, pDG (R484E/R585E and pDP8.ape).

In some embodiments, a plasmid comprising the transgene is combined with one or more helper plasmids, e.g., that contain a rep gene of a first serotype and a cap gene of the same serotype or a different serotype, and transfected into helper cells such that the rAAV is packaged.

In some embodiments, the packaging is performed in a helper cell or producer cell, such as a mammalian cell or an insect cell. Exemplary mammalian cells include, but are not limited to, HEK293 cells, COS cells, HeLa cells, BHK cells, or CHO cells (see, e.g., ATCC® CRL-1573™, ATCC® CRL-1651™, ATCCR CRL-1650™, ATCC® CCL-2, ATCC® CCL-10™, or ATCC® CCL-61™). Exemplary insect cells include, but are not limited to Sf9 cells (see, e.g., ATCC® CRL-1711™). The helper cell may comprise rep and/or cap genes that encode the Rep protein and/or Cap proteins for use in a method described herein. In some embodiments, the packaging is performed in vitro.

Various methods are known in the art relating to the production and purification of AAV vectors. See, e.g., Mizukami, Hiroaki, et al. A Protocol for AAV vector production and purification; U.S. Patent Publication Numbers US20070015238 and US20120322861. For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV can be packaged and subsequently purified.

### Pharmaceutical Compositions

Exemplary pharmaceutical compositions comprising the vectors described herein are detailed below.

Pharmaceutical acceptable carriers are determined in part by the particular composition being administered, as well as by the particular method used to administer the compositions. Accordingly, there is a wide variety of suitable formulations of pharmaceutical compositions available.

Formulations for both ex vivo and in vivo administrations include suspensions in liquid or emulsified liquids. The active ingredients often are mixed with excipients which are pharmaceutically acceptable and compatible with the active ingredient. Suitable excipients include, for example, water, saline, dextrose, glycerol, ethanol, or the like, and combinations thereof. In addition, the compositions may contain minor amounts of auxiliary substances, such as wetting or emulsifying agents, pH buffering agents, stabilizing agents or other reagents that enhance the effectiveness of the pharmaceutical composition.

In some embodiments, a pharmaceutical composition comprising a rAAV vector described herein is provided. The pharmaceutical composition containing a rAAV vector or particle of the invention contains a pharmaceutically acceptable excipient, diluent or carrier. Examples of suitable pharmaceutical carriers are well known in the art and include phosphate buffered saline solutions, water, emulsions, such as oil/water emulsions, various types of wetting agents, sterile solutions and the like. Such carriers can be formulated by conventional methods and are administered to the subject at a therapeutically effective amount.

### Therapeutic Applications and Methods of Use

The transgenes and rAAV vectors of the present disclosure can be used to treat a subject who has Fabry disease. Accordingly, the vectors of the present disclosure can be used to treat a subject who has Fabry disease, and as such reduce one or more symptoms associated with the disease. In some embodiments, the vectors of the present disclosure can be used to treat a subject who has reduced expression or no expression of α-GAL, and/or impaired activity of α-GAL enzyme.

Non-limiting examples of Fabry symptoms include neuropathic pain, hypohidrosis or anhidrosis, exercise intolerance, abdominal cramps, diarrhea, angiokeratoma, verticillata, tinnitus, proteinuria, chronic kidney disease, hypertension, coronary insufficiency, AV conduction disturbances, arrhythmias and valvular malfunction, left ventricular hypertrophy, seizure and stroke.

The neuropathy of Fabry disease includes neuropathic pain, reduced cold and warm sensation (i.e., thermal hypoesthesia), hearing loss, and possibly, gastrointestinal disturbance. The neuropathy could be caused by peripheral abnormalities of peripheral nerve fibers particularly small nerve fibers (myelinated and unmyelinated nerve fibers). The impairments are partially due to accumulation of GB3 in the peripheral nervous system such as DRG.

In various embodiments described herein, the rAAV vectors according to the present disclosure are used to treat peripheral neuropathy and/or one or more symptoms associated with peripheral neuropathy in a patient diagnosed with Fabry disease. The patient has or is developing one or more peripheral neuropathy related symptoms such as neuropathic pain. Peripheral neuropathic pain in patients with Fabry disease can manifest as chronic, burning pain and superimposed attacks of acute excruciating pain, dysesthesias, thermal sensation deficits (primarily cold perception), and paresthesias (e.g., painless tingling). Symptoms related to autonomic nervous system dysfunction may include hypohidrosis, impaired pupillary constriction and saliva and tear production, gastrointestinal dysmotility (abdominal cramping pain, bloating, diarrhea, nausea), and sensory losses.

The rAAV vectors expressing the GLA transgene as described herein are particularly suitable to alleviate the symptoms associated with Fabry disease. In some embodiments, the rAAV vectors expressing the GLA transgene can be used to alleviate peripheral neuropathy in a subject diagnosed with Fabry disease. The method comprises administering to the subject a composition comprising the rAAV expressing α-GAL enzyme or a variant thereof, as described herein. The method alleviates the neuropathy manifestations including neuropathic pain, reduced cold and warm sensation (i.e., thermal hypoesthesia), hearing loss, other sensory deficiency, and possibly, gastrointestinal disturbance.

In some embodiments, the rAAV vector described herein is used to improve peripheral abnormalities in nerve fibers in Fabry disease. The peripheral nerves are small nerve fibers including myelinated and unmyelinated nerve fibers. The nerve fibers are cutaneous sensory nerves. In some embodiments, the treatment with the vector can preserve the density of nerve fibers in the peripheral nervous system.

In some embodiments, the rAAV vector is used to reduce the accumulation of GB3 and lysoGb3 in the peripheral nervous system. In some embodiments, the morphological deficiencies in the DRG are improved including decreased expression of surrogate biomarker LAMP1 upon administration of the rAAV vector as described herein. LAMP 1 (lysosomal associated membrane protein 1) is a biomarker for neurodegeneration. Patients with Fabry disease have severely enlarged dorsal root ganglia with dysfunctional perfusion, which may be due to glycolipid accumulation in the dorsal root ganglia mediating direct neurotoxic effects and decreased neuronal blood supply. The increased exposure of α-GAL enzyme could decrease LAMP1 in the DRG.

In some embodiments, the rAAV vector for the GLA transgene results in the reduction of globotriaosylceramide (GB3) in the subject. In some embodiments, GB3 in the subject is reduced by about 95%, 90%, 85%, 80%, 75%, 70%, 65%, 60%, 55%, 50%, 45%, 40%, 35%, 30%, 25%, 20%, 15%, or about 10% in comparison to the subject's baseline GB3 level prior to administering the rAAV comprising GLA. Accordingly, in some embodiments, the administered rAAV comprising GLA reduces GB3 in the subject by about 95%. In some embodiments, the administered rAAV comprising GLA reduces GB3 in the subject by about 90%. In some embodiments, the administered rAAV comprising GLA reduces GB3 in the subject by about 85%. In some embodiments, the administered rAAV comprising GLA reduces GB3 in the subject by about 80%. In some embodiments, the administered rAAV comprising GLA reduces GB3 in the subject by about 75%. In some embodiments, the administered rAAV comprising GLA reduces GB3 in the subject by about 70%. In some embodiments, the administered rAAV comprising GLA reduces GB3 in the subject by about 65%. In some embodiments, the administered rAAV comprising GLA reduces GB3 in the subject by about 60%. In some embodiments, the administered rAAV comprising GLA reduces GB3 in the subject by about 55%. In some embodiments, the administered rAAV comprising GLA reduces GB3 in the subject by about 50%. In some embodiments, the administered rAAV comprising GLA reduces GB3 in the subject by about 45%. In some embodiments, the administered rAAV comprising GLA reduces GB3 in the subject by about 40%. In some embodiments, the administered rAAV comprising GLA reduces GB3 in the subject by about 35%. In some embodiments, the administered rAAV comprising GLA reduces GB3 in the subject by about 30%. In some embodiments, the administered rAAV comprising GLA reduces GB3 in the subject by about 25%. In some embodiments, the administered rAAV comprising GLA reduces GB3 in the subject by about 20%. In some embodiments, the administered rAAV comprising GLA reduces GB3 in the subject by about 15%. In some embodiments, the administered rAAV comprising GLA reduces GB3 in the subject by about 10%.

In some embodiments, GB3 in the serum is reduced by about 95%, 90%, 85%, 80%, 75%, 70%, 65%, 60%, 55%, 50%, 45%, 40%, 35%, 30%, 25%, 20%, 15%, or about 10% in comparison to the subject's baseline GB3 level prior to administering the rAAV expressing the GLA transgene. In some embodiments, GB3 in the kidney, heart and/or liver is reduced by about 95%, 90%, 85%, 80%, 75%, 70%, 65%, 60%, 55%, 50%, 45%, 40%, 35%, 30%, 25%, 20%, 15%, or about 10% in comparison to the subject's baseline GB3 level prior to administering the rAAV expressing the GLA transgene.

In some embodiments, the rAAV vector is used to reduce the accumulation of GB3 and lysoGb3 in the kidney. In some embodiments, the rAAV vector is used to reduce the accumulation of GB3 and lysoGb3 in the liver. In some embodiments, the rAAV vector is used to reduce the accumulation of GB3 and lysoGb3 in the heart. In some embodiments, the rAAV vector is used to reduce the accumulation of GB3 and lysoGb3 in the serum.

In some embodiments, the administered rAAV comprising GLA leads to production of α-GAL enzyme in the kidney of a subject. In some embodiments, the administered rAAV comprising GLA leads to production of α-GAL enzyme in the heart of the subject. In some embodiments, the administered rAAV comprising GLA leads to production of α-GAL enzyme in the liver of a subject. In some embodiments, the administered rAAV comprising GLA leads to production of α-GAL enzyme in the serum of a subject.

In some embodiments, LAMP1 levels in the peripheral nervous system (e.g., in the DRG) is reduced by about 95%, 90%, 85%, 80%, 75%, 70%, 65%, 60%, 55%, 50%, 45%, 40%, 35%, 30%, 25%, 20%, 15%, or about 10% in comparison to the subject's baseline LAMP1 expression prior to administering the rAAV expressing the GLA transgene. In some embodiments, the lysosomal-autophagy organelles in the DRG are reduced by the rAAV gene therapy.

In some embodiments, the administered rAAV comprising a GLA transgene reduces GB3 in the subject for at least about 2 weeks, 1 month, 2 months, 3 months, 4 months, 5 months, 6 months, 12 months, 1 year, 2 years, 3 years, 4 years, 5 years, or more than 5 years.

In some embodiments, the vector mediated gene therapy provides consistent expression of α-GAL and high α-GAL activity in serum and other tissues (e.g., kidney, heart and liver and the nervous system). In some embodiments, the vector mediated gene therapy provides consistent expression of α-GAL and high α-GAL activity in the peripheral nervous system (e.g., DRG).

In some examples, functional α-GAL is detectable in serum and tissues of the subject about 2 to 18 weeks post administration of the rAAV vector. In some embodiments, functional α-GAL is detectable in serum and tissues of the subject 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, or 18 weeks post administration of the rAAV vector. In some embodiments, functional α-GAL is detectable in serum and tissues of the subject at least 3 months, 6 months, 12 months, 2 years, 3 years, 4 years, 5 years, 6 years, 7 years, 8 years, 9 years, 10 years, 15 years or 20 years or longer after administration of the rAAV vector.

In some embodiments, functional α-GAL is detectable in the nervous system (e.g., DRG) of the subject about 2 to 18 weeks post administration of the rAAV vector. In some embodiments, functional α-GAL is detectable in serum and tissues of the subject 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, or 18 weeks or longer post administration of the rAAV vector. In some embodiments, functional α-GAL is detectable in the nervous system (e.g., DRG) of the subject at least 3 months, 6 months, 12 months, 2 years, 3 years, 4 years, 5 years, 6 years, 7 years, 8 years, 9 years, 10 years, 15 years or 20 years after administration of the rAAV vector.

In some embodiments, following administration of the AAV vector to the subject the levels of functional α-GAL detectable in the circulation are between about 2 and 1000 fold or higher than 10 fold, higher than 20 fold, higher than 30 fold, higher than 40 fold, higher than 50 fold, higher than 60 fold, higher than 70 fold, higher than 80 fold, higher than 90 fold, higher than 95 fold, or 100 fold or more greater than the amount of functional α-GAL detectable in the subject before administration of the rAAV comprising GLA transgene.

In some embodiments, following administration of the AAV vector to the subject the levels of functional α-GAL detectable in the PNS are between about 2 and 100 fold or higher than 10 fold, higher than 20 fold, higher than 30 fold, higher than 40 fold, higher than 50 fold, higher than 60 fold, higher than 70 fold, higher than 80 fold, higher than 90 fold, higher than 95 fold, or 100 fold or more greater than the amount of functional α-GAL detectable in the subject before administration of the rAAV comprising GLA transgene.

In some embodiments, following administration of the AAV vector to the subject the level of detectable active α-GAL meets or exceeds human therapeutic level, i.e., level of α-GAL considered to be normal circulating level in humans (e.g., 5-9 nmol/hour/ml). In some embodiments, the levels of active α-GAL post administration of the rAAV vector is about between 2 and 35 times or greater than 35 times, greater than 40 times, greater than 45 times, greater than 50 times, greater than 55 times, greater than 60 times, greater than 65 times, greater than 70 times greater than 75 times greater than 80 times greater than 85 times, greater than 90 times, greater than 95 times, greater than 100 times, greater than 200 times, greater than 300 times, greater than 400 times, greater than 500 times, greater than 600 times, greater than 700 times, greater than 800 times, greater than 900 times, or greater than 1000 times the human therapeutic level.

Thus, administration of rAAV vector comprising a GLA transgene results in sustained robust expression in comparison to a single administration of purified α-GAL to a subject in need.

In some embodiments, following administration of the AAV vector to the subject, 10% Gb3 reduction in ERT resistant cell types is achieved. In some embodiments, following administration of the AAV vector to the subject, 15% Gb3 reduction in ERT resistant cell types is achieved. In some embodiments, following administration of the AAV vector to the subject, 20% Gb3 reduction in ERT resistant cell types is achieved. In some embodiments, following administration of the AAV vector to the subject, 25% Gb3 reduction in ERT resistant cell types is achieved. In some embodiments, following administration of the AAV vector to the subject, 30% Gb3 reduction in ERT resistant cell types is achieved. In some embodiments, following administration of the AAV vector to the subject, 35% Gb3 reduction in ERT resistant cell types is achieved. In some embodiments, following administration of the AAV vector to the subject, 40% Gb3 reduction in ERT resistant cell types is achieved. In some embodiments, following administration of the AAV vector to the subject, 45% Gb3 reduction in ERT resistant cell types is achieved. In some embodiments, following administration of the AAV vector to the subject, 50% Gb3 reduction in ERT resistant cell types is achieved. In some embodiments, following administration of the AAV vector to the subject 55% Gb3 reduction in ERT resistant cell types is achieved. In some embodiments, following administration of the AAV vector to the subject, 60% Gb3 reduction in ERT resistant cell types is achieved. In some embodiments, following administration of the AAV vector to the subject, 65% Gb3 reduction in ERT resistant cell types is achieved.

In some embodiments, the amelioration of Fabry symptoms is sustained for at least 1 week. In some embodiments, the amelioration of Fabry symptoms is sustained for at least 2 weeks. In some embodiments, the amelioration of Fabry symptoms is sustained for at least 3 weeks. In some embodiments, the amelioration of Fabry symptoms is sustained for at least 4 weeks. In some embodiments, the amelioration of Fabry symptoms is sustained for at least 1 month. In some embodiments, the amelioration of Fabry symptoms is sustained for at least 2 months. In some embodiments, the amelioration of Fabry symptoms is sustained for at least 3 months. In some embodiments, the amelioration of Fabry symptoms is sustained for at least 4 months. In some embodiments, the amelioration of Fabry symptoms is sustained for at least 5 months. In some embodiments, the amelioration of Fabry symptoms is sustained for at least 6 months. In some embodiments, the amelioration of Fabry symptoms is sustained for at least 7 months. In some embodiments, the amelioration of Fabry symptoms is sustained for at least 8 months. In some embodiments, the amelioration of Fabry symptoms is sustained for at least 9 months. In some embodiments, the amelioration of Fabry symptoms is sustained for at least 10 months. In some embodiments, the amelioration of Fabry symptoms is sustained for at least 11 months. In some embodiments, the amelioration of Fabry symptoms is sustained for at least 12 months. In some embodiments, the amelioration of Fabry symptoms is sustained for at least 1 year. In some embodiments, the amelioration of Fabry symptoms is sustained for 1 year or more.

In some embodiments, following administration of the AAV vector to the subject, 10% Gb3 reduction in ERT resistant cell types is achieved at 6 months. In some embodiments, following administration of the AAV vector to the subject, 15% Gb3 reduction in ERT resistant cell types is achieved at 6 months. In some embodiments, following administration of the AAV vector to the subject, 20% Gb3 reduction in ERT resistant cell types is achieved at 6 months. In some embodiments, following administration of the AAV vector to the subject, 25% Gb3 reduction in ERT resistant cell types is achieved at 6 months. In some embodiments, following administration of the AAV vector to the subject, 30% Gb3 reduction in ERT resistant cell types is achieved at 6 months. In some embodiments, following administration of the AAV vector to the subject, 35% Gb3 reduction in ERT resistant cell types is achieved at 6 months. In some embodiments, following administration of the AAV vector to the subject, 40% Gb3 reduction in ERT resistant cell types is achieved at 6 months. In some embodiments, following administration of the AAV vector to the subject, 45% Gb3 reduction in ERT resistant cell types is achieved at 6 months. In some embodiments, following administration of the AAV vector to the subject, 50% Gb3 reduction in ERT resistant cell types is achieved at 6 months.

In some embodiments, following administration of the AAV vector to the subject, 30% Gb3 reduction in ERT resistant cell types is achieved at 1 months. In some embodiments, following administration of the AAV vector to the subject, 30% Gb3 reduction in ERT resistant cell types is achieved at 2 months. In some embodiments, following administration of the AAV vector to the subject, 30% Gb3 reduction in ERT resistant cell types is achieved at 3 months. In some embodiments, following administration of the AAV vector to the subject, 30% Gb3 reduction in ERT resistant cell types is achieved at 4 months. In some embodiments, following administration of the AAV vector to the subject, 30% Gb3 reduction in ERT resistant cell types is achieved at 5 months. In some embodiments, following administration of the AAV vector to the subject, 30% Gb3 reduction in ERT resistant cell types is achieved at 6 months. In some embodiments, following administration of the AAV vector to the subject, 30% Gb3 reduction in ERT resistant cell types is achieved at 7 months. In some embodiments, following administration of the AAV vector to the subject, 30% Gb3 reduction in ERT resistant cell types is achieved at 8 months. In some embodiments, following administration of the AAV vector to the subject, 30% Gb3 reduction in ERT resistant cell types is achieved at 9 months.

The rAAV vector is administered to a subject diagnosed with Fabry disease via a suitable route. In some embodiments, the rAAV vector is administered by intravenous, intraperitoneal, subcutaneous, or intradermal administration. In some embodiments, the rAAV vector is administered intravenously. In some embodiments, the intradermal administration comprises administration by use of a “gene gun” or biolistic particle delivery system. In some embodiments, the rAAV vector is administered via a non-viral lipid nanoparticle. For example, a composition comprising the rAAV vector may comprise one or more diluents, buffers, liposomes, a lipid, a lipid complex. In some embodiments, the rAAV vector is comprised within a microsphere or a nanoparticle, such as a lipid nanoparticle.

In some embodiments, the rAAV vector is administered by spinal applications, e.g., spinal injection. In some embodiments, the rAAV vector remains episomal following administration to a subject in need thereof. In some embodiments, the rAAV vector does not remain episomal following administration to a subject in need thereof. For example, in some embodiments, the rAAV vector integrates into the genome of the subject. Such integration can be achieved, for example, by using various gene-editing technologies, such as, zinc finger nucleases (ZFNs), Transcription activator-like effector nucleases (TALENS), ARCUS genome editing, and/or CRISPR-Cas systems.

In some embodiments, the rAAV vector comprising a GLA transgene is administered to a subject in need as a single dose. In some embodiments, the rAAV vector is administered to the subject with the minimal effective dose (MED). As used herein, MED refers to the rAAV GLA vector dose required to achieve α-GAL activity resulting in reduced GB3 levels in a subject.

In some embodiments, the dosage is at a dose ranging from 1×10¹⁰ vg/kg (viral genome/kilogram of body weight) to 1×10¹⁴ vg/kg. In some embodiments, the rAAV vector of the present disclosure is administered at a dose ranging from 1×10¹⁰ vg/kg to 5×10¹³ vg/kg. In some embodiments, the rAAV vector of the present disclosure is administered at a dose ranging from 1×10¹⁰ vg/kg to 1×10¹³ vg/kg. In some embodiments, the rAAV vector of the present disclosure is administered at a dose ranging from 1×10¹⁰ vg/kg to 5×10¹² vg/kg. In some embodiments, the rAAV vector of the present disclosure is administered at a dose ranging from 1×10¹⁰ vg/kg to 1×10¹² vg/kg. In some embodiments, the rAAV vector of the present disclosure is administered at a dose ranging from 1×10¹⁰ vg/kg to 5×10¹¹ vg/kg. In some embodiments, the rAAV vector of the present disclosure is administered at a dose ranging from 1×10¹⁰ vg/kg to 2.5×10¹¹ vg/kg. In some embodiments, the rAAV vector of the present disclosure is administered at a dose ranging from 5×10¹⁰ vg/kg to 1×10¹⁴ vg/kg. In some embodiments, the rAAV vector of the present disclosure is administered at a dose ranging from 5×10¹⁰ vg/kg to 5×10¹³ vg/kg. In some embodiments, the rAAV vector of the present disclosure is administered at a dose ranging from 5×10¹⁰ vg/kg to 1×10¹³ vg/kg. In some embodiments, the rAAV vector of the present disclosure is administered at a dose ranging from 5×10¹⁰ vg/kg to 5×10¹² vg/kg. In some embodiments, the rAAV vector of the present disclosure is administered at a dose ranging from 5×10¹⁰ vg/kg to 1×10¹² vg/kg. In some embodiments, the rAAV vector of the present disclosure is administered at a dose ranging from 5×10¹⁰ vg/kg to 5×10¹¹ vg/kg. In some embodiments, the rAAV vector of the present disclosure is administered at a dose ranging from 5×10¹⁰ vg/kg to 2.5×10¹¹ vg/kg.

In some embodiments, the rAAV vector of the present disclosure is administered at a dose of 1.5×10¹⁰ to 2.5×10¹⁰ vg/kg. In some embodiments, the rAAV vector of the present disclosure is administered at a dose of 1×10¹⁰ to 2×10¹⁰ vg/kg. In some embodiments, the rAAV vector of the present disclosure is administered at a dose of 2.5×10¹⁰ to 3.5×10¹⁰ vg/kg. In some embodiments, the rAAV vector of the present disclosure is administered at a dose of 2.5×10¹⁰ vg/kg. In some embodiments, the rAAV vector of the present disclosure is administered at a dose of 3.5×10¹⁰ to 4.5×10¹⁰ vg/kg. In some embodiments, the rAAV vector of the present disclosure is administered at a dose of 4.5×10¹⁰ to 5.5×10¹⁰ vg/kg. In some embodiments, the rAAV vector of the present disclosure is administered at a dose of 5.5×10¹⁰ to 6.5×10¹ vg/kg. In some embodiments, the rAAV vector of the present disclosure is administered at a dose of 6.5×10¹⁰ to 7.5×10¹⁰ vg/kg. In some embodiments, the rAAV vector of the present disclosure is administered at a dose of 7.5×10¹⁰ to 8.5×10¹⁰ vg/kg. In some embodiments, the rAAV vector of the present disclosure is administered at a dose of 8.5×10¹⁰ to 9.5×10¹⁰ vg/kg. In some embodiments, the rAAV vector of the present disclosure is administered at a dose of 9.5×10¹⁰ to 1×10¹¹ vg/kg. In some embodiments, the rAAV vector of the present disclosure is administered at a dose of 1×10^{1 1}vg/kg. In some embodiments, the rAAV vector of the present disclosure is administered at a dose from 1×10¹¹ to 1.5×10¹¹ vg/kg. In some embodiments, the rAAV vector of the present disclosure is administered at a dose from 1.5×10¹¹ to 2.5×10¹¹ vg/kg. In some embodiments, the rAAV vector of the present disclosure is administered at a dose of 2.5×10¹¹ vg/kg. In some embodiments, the rAAV vector of the present disclosure is administered at a dose from 2×10¹¹ to 2.5×10¹¹ vg/kg. In some embodiments, the rAAV vector of the present disclosure is administered at a dose from 2.5E11 to 3.5×10¹¹ vg/kg. In some embodiments, the rAAV vector of the present disclosure is administered at a dose from 3.5×10¹¹ to 4.5×10¹¹ vg/kg. In some embodiments, the rAAV vector of the present disclosure is administered at a dose from 4.5×1011 to 5.5×10¹¹ vg/kg. In some embodiments, the rAAV vector of the present disclosure is administered at a dose from 5.5×10¹¹ to 6.5×10¹¹ vg/kg. In some embodiments, the rAAV vector of the present disclosure is administered at a dose from 6.5×10¹¹ to 7.5×10¹¹ vg/kg. In some embodiments, the rAAV vector of the present disclosure is administered at a dose from 7.5×10¹¹ to 8.5×10¹¹ vg/kg. In some embodiments, the rAAV vector of the present disclosure is administered at a dose from 8.5×10¹¹ to 9.5×10¹¹ vg/kg. In some embodiments, the rAAV vector of the present disclosure is administered at a dose from 9.5×10¹¹ to 1×10¹² vg/kg. In some embodiments, the rAAV vector of the present disclosure is administered at a dose from 1×10¹² vg/kg to 2×10¹². In some embodiments, the rAAV vector of the present disclosure is administered at a dose from 1.5×10¹² vg/kg to 2.5×10¹². In some embodiments, the rAAV vector of the present disclosure is administered at a dose from 2.5×10¹² vg/kg to 3.5×10¹². In some embodiments, the rAAV vector of the present disclosure is administered at a dose from 3.5×10¹² vg/kg to 4.5×10¹². In some embodiments, the rAAV vector of the present disclosure is administered at a dose from 4.5×10¹² vg/kg to 5.5×10¹².

In some embodiments, the rAAV vector of the present disclosure is administered at a dose of 2.5×1010 vg/kg to 1×1011 vg/kg. In some embodiments, the rAAV vector of the present disclosure is administered at a dose of 2.5×1010 vg/kg to 2.5×1012 vg/kg.

In some embodiments, a rAAV gene therapy vector expressing an alpha-galactosidase protein as described herein is administered to a subject at a lower or an equivalent dose used in case of other gene therapy vectors (e.g., a AAV with a liver specific promoter); however, still exhibits higher serum and tissue exposure, e.g., high and sustained exposure in the PNS (e.g., DRG).

In some embodiments, the rAAV vectors expressing a GLA transgene provided herein are used as a prophylactic treatment in a subject who has Fabry disease to prevent onset and/or progression of one or more symptoms in Fabry disease. In one embodiment, the rAAV vectors expressing a GLA transgene provided herein are used as a prophylactic treatment in a subject who has Fabry disease to prevent onset and/or progression of one or more periphery neuropathy manifestations.

Prophylactic treatment may be administered, for example, to a subject who is not yet ill, but who is susceptible to, or otherwise at risk of, a particular biological condition, including Fabry disease (e.g., the subject may have mutations that cause Fabry disease but is asymptomatic or the status of mutations that cause Fabry disease is unknown). In some embodiments, therapeutic treatment may be administered, for example, to a subject already suffering from Fabry disease in order to improve or stabilize the subject's condition (e.g., a patient already presenting symptoms of Fabry disease).

### Peripheral Neuropathy

In some embodiments, the present method further includes evaluating methods and tests for evaluating improvement of peripheral neuropathy in Fabry patients treated with the rAAV mediated gene therapy contemplated in the present disclosure.

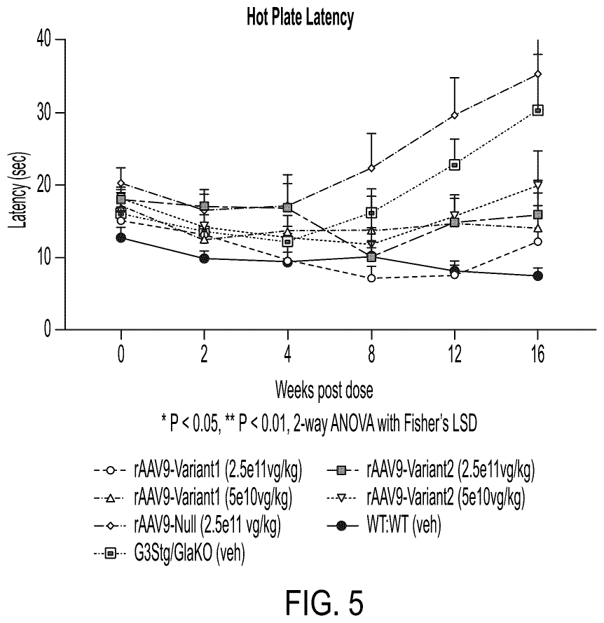

As discussed in the present disclosure, the hot plate latency test in Fabry disease mouse model shows that G3Stg/GLAko mice treated with rAAV vector comprising a GLA transgene significantly responded to the heat stimulus with similar sensitivity to wild type mice (e.g., FIG. 5). The results observed in the mice suggest that a similar amelioration of thermal hyposensitivity in Fabry patients may be achieved by the rAAV mediated GLA gene therapy. Accordingly, peripheral neuropathy in Fabry patients can be measured using clinic methods commonly used to assess the efficiency of the rAAV mediated GLA gene therapy as described herein.

Fabry patients can be diagnosed with development of neuropathy associated symptom at young ages. In some cases, patients can be diagnosed with development of neuropathy associated symptom at adulthood. Doctors (e.g., neurologists) in general can recognize an array of distinct early features of Fabry disease of which several are related to the small fiber neuropathy. The features include chronic burning pain, attacks of excruciating pain, Paresthesias/dysesthesias, sensory loss, Hypohidrosis/anhidrosis, Abdominal cramp, (post-prandial) diarrhea, bloating, nausea and Tinnitus, hearing loss.

A neuropathy pain assessment scale may be used at the initial assessment and follow up examinations after treatment. The Neuropathy Symptoms and Change Questionnaire evaluates the number, severity and change of symptoms, as well as motor, autonomic, large fiber and small fiber sensory nerve function (Dyck et al.,: Longitudinal assessment of diabetic polyneuropathy using a composite score in the Rochester Diabetic Neuropathy Study cohort. Neurology. 1997, 49:229-239). Other available neuropathic pain evaluation tools include the Leeds Assessment of Neuropathic Symptoms and Signs, Neuropathic Pain Questionnaire, Neuropathic Pain Symptom Inventory, Douleur Neuropathique en 4 questions, pain DETECT, and ID-pain. The Total Symptoms Score has been used for grading of neuropathic pain in clinical studies in diabetes and Fabry disease patients with peripheral neuropathy.

A neurologic examination of sensations mediated by peripheral small fibers may be performed before and after the treatment. For example, A rather crude test of thermal perception may be used, which consists of placing tubes filled with cold and warm water, a cold and warm tuning fork, the handle of a reflex hammer, or thermal discs with a polyvinyl surface on one side and a metal surface on the other side (similar to the “Minnesota Thermal Discs” on the patient's foot.

Pain perception and hyperalgesia in a patient before and after the treatment can be tested by applying just enough pressure with a sharp pin to indent the skin. Loss of discrimination between pinprick sensation and application of a blunt pressure sensation points towards small fiber neuropathy.

In some embodiments, a Fabry patient who has or is developing one or more neuropathy associated symptoms is treated with the rAAV vector expression a GLA transgene as described herein. The improvement of neuropathy associated symptoms are assessed following the same assessment after treatment. In some examples, the assessment is performed 2 weeks, 5 weeks, 10 weeks, 18 weeks, 6 months, or longer after the treatment.

In some embodiments, the peripheral neuropathy is quantified by the Computer Evaluated Sensory Evaluator as described by Dyck and O'Brien (Quantitative sensation testing in epidemiological and therapeutic studies of peripheral neuropathy. Muscle Nerve. 1999; 22:659-662) and Schiffmann, et al. (Enzyme replacement therapy improves peripheral nerve and sweat function in Fabry disease. Muscle Nerve. 2003; 28:703-710); the contents of each of which are incorporated herein by reference in their entirety.

In some embodiments, the cold and warmth perception of the patient being treated with the present method is measured. Cold perception in the feet is the predominant marker for Fabry disease neuropathy and may be quantified in units of just noticeable difference (JND) on a scale from 1 to 25 for evaluating the treatment outcomes (see Ries et al., 2007). In the context of experimental measurement focusing on the sensation and perception, the term “just-noticeable difference” or “JND” is the amount something must be changed in order for a difference to be noticeable, detectable at least half the time.

In some embodiments, quantitative sensory test (QST) may be used to investigate patient's neuropathy improvement; the QST may be performed following standard procedure (Rolke et al., Pain. 2006; 123:231-243; the contents of which are incorporated herein by reference in their entirety). In some embodiments, a calibrated thermode may be used for QST before and after the rAAV treatment.

In some embodiments, the morphology of peripheral nerve fibers are imaged and measured for the treatment outcomes.

In some embodiments, skin biopsies are taken from patients for assessment of cutaneous nerve fiber density. For example, skin specimens are obtained from the lower leg and processed for standard immunostaining to assess nerve fiber morphology.

In some embodiments, neuronal conduction activity may be measured before and after the rAAV treatment. As a non-limiting example, microneurography recording may be used to record action potentials of human nerve fibers (e.g., skin fibers).

### Combination Therapy

The compositions and methods of the invention can also be used in conjunction with other remedies known in the art that are used to treat Fabry disease or its complications, including but not limited to: SRT (Substrate Reduction Therapy), ERT (e.g., agalsidase beta), pain relief medications (e.g., lidocaine, diphenylhydantoin, carbamazepine, gabapentin, phenytoin, neurotropin, opioids); dyspepsia treatment (e.g., metoclopramide, H-2 blockers), vitamin D replacements etc., beta blockers (metoprolol, Acebutolol, bisoprolol, atenolol, propranolol, etc.) anti-coagulation treatment (Heparin, warfarin, Apixaban, Rivaroxaban).

The compositions and methods of the invention can also be used in conjunction with other forms of treatment including but not limited to: physical exercise (e.g. dialysis, kidney transplantation); dietary salt restriction, fiber intake, installation of a pacemaker, and cardiac transplantation.

### EXAMPLES

Exemplary features, objects, and advantages of the present invention are apparent in the examples that follow. It should be understood, however, that the examples, while indicating embodiments of the present invention, are given by way of illustration only, not limitation. Various changes and modifications within the scope of the invention will become apparent to those skilled in the art from the examples.

### Example 1. Production and Purification Viral Vectors Expressing α-GAL Enzyme

This example summarizes viral vectors encompassed by the present disclosure.

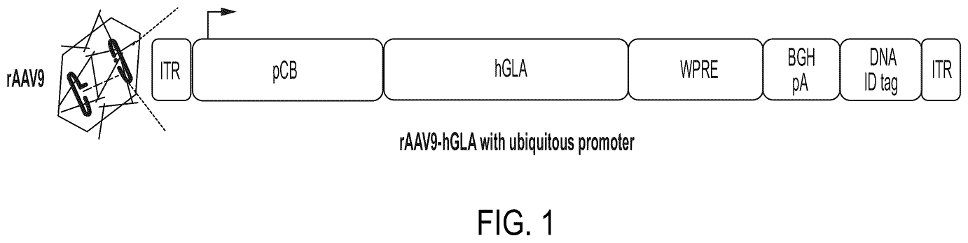

A recombinant adeno-associated virus 9 (rAAV9) was developed to express wild type human α-GAL or α-GAL variants (e.g., amino acid sequences shown in Table 1) under the control of a ubiquitous promoter, in a viral vector. A WPRE element was linked to the 3′ end of the GLA transgene to increases transgene expression to improve mRNA stability. A bovine growth hormone poly A tail was appended to the 3′ end of the WPRE element. The DNA construct of promoter-GLA-WPRE-BGHpA was integrated between the inverted terminal repeats of a circular plasmid vector. FIG. 1 shows an exemplary rAAV9 vector construct.

rAAV vectors were encapsulated using the AAV2 inverted terminal repeats and rep sequences using methods in the art. The rAAV9 stocks were produced using HEK-293T cells by the adenovirus free, triple-plasmid co-transfection method and purified using cesium chloride ultracentrifugation. Titers of v.g. particle number were determined by quantitative PCR.

Purified rAAV9 virus suspensions were diluted in the formulation buffer consisting of 1.5 mM KH₂PO₄ (Potassium dihydrogen phosphate), 2.7 mM KCl (Potassium chloride), 8.1 mM Na₂HPO₄ (Di-sodium hydrogen phosphate), 136.9 mM NaCl (Sodium chloride) and 0.001% Pluronic F-68. Null vector with rAAV9 capsid (rAAV9-null) were used as controls.

Two rAAV vectors Variant 1 and Variant 2 were prepared and used for the following studies. The diagram of the transgene expression cassette is shown in FIG. 1. The Variant 1 comprising a codon optimized nucleic acid sequence of SEQ ID NO: 12. Variant 2 comprising a codon optimized nucleic acid sequence of SEQ ID NO: 13.

### Example 2. α-GAL Activity in Serum Following Administration

This Example shows that Variant 1 and Variant 2 provided α-GAL protein expression in serum for at least 18 weeks following administration of vectors into mice.

An aggravated Fabry mouse model (G3Stg/GlaKO), generated by crossing Gla knockout (GlaKO) mice with transgenic mice expressing human Gb3 synthase (G3Stg) to increase Gb3 accumulation was used for this study. These mice have up to 10-fold higher levels of substrate GB3 in various tissues compared to age matched GlaKO mice, mirroring symptoms observed in Fabry patients.

8-12 weeks old male G3Stg/GLAko mice were divided into groups (n=6-12/each group) and administered the purified rAAV9 vectors expressing Variant 1 and Variant 2 intravenously (IV) at two different doses, 2.5×10¹¹, 5×10¹⁰ vg/kg and the mice were followed for 18 weeks. A rAAV9-Null vector (Control) was administered to a group of G3Stg/GLAko mice at 2.5×10¹¹ vg/kg as a negative control. Vehicle treated wild type (WT:WT) group was used as controls as well. The study design is summarized in Table 4 below.

G3Stg/GLAko mice showed a marked decrease in body weight over the course of the study; mice treated with rAAV9 vectors expressing Variant 1 or Variant 2 had higher body weights compared with mice given a null AAV vector or vehicle only (data not shown).

Serum was collected at multiple time-points during the study as well as at the end of 18-weeks.

TABLE 4
Study design

| Group | | Group | |
|---|---|---|---|
| # | Treatment | size | Dose and route |
| 1 | G3Stg/GLA KO-rAAV9-Variant 1 | 11 | 2.5e11 vg/kg; IV |
| 2 | G3Stg/GLA KO-rAAV9-Variant 2 | 11 | 2.5e11 vg/kg; IV |
| 3 | G3Stg/GLA KO-rAAV9-Variant 1 | 11 | 5.0e10 vg/; IV |
| 4 | G3Stg/GLA KO-rAAV9-Variant 2 | 11 | 5.0e10 vg/kg; IV |
| 5 | G3Stg/GLA KO-rAAV9- null | 10 | 2.5e11 vg/kg; IV |
| 6 | WT:WT Vehicle | 12 | 0; IV |

The quantity of α-GAL in the serum at 18 weeks after administration was measured.

### Example 3. α-GAL Activity in Serum and Different Tissues Following Vector Administration

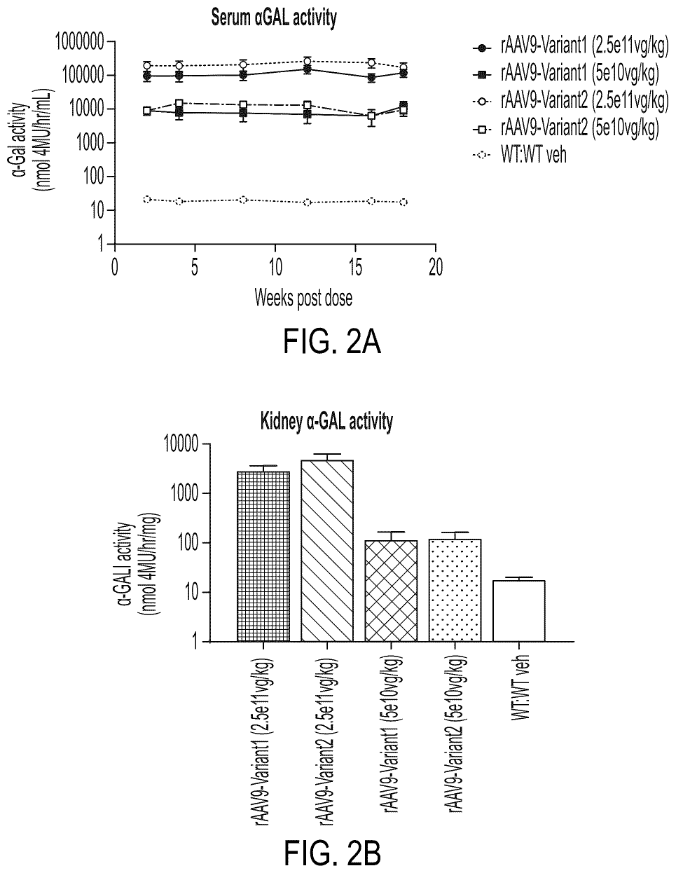

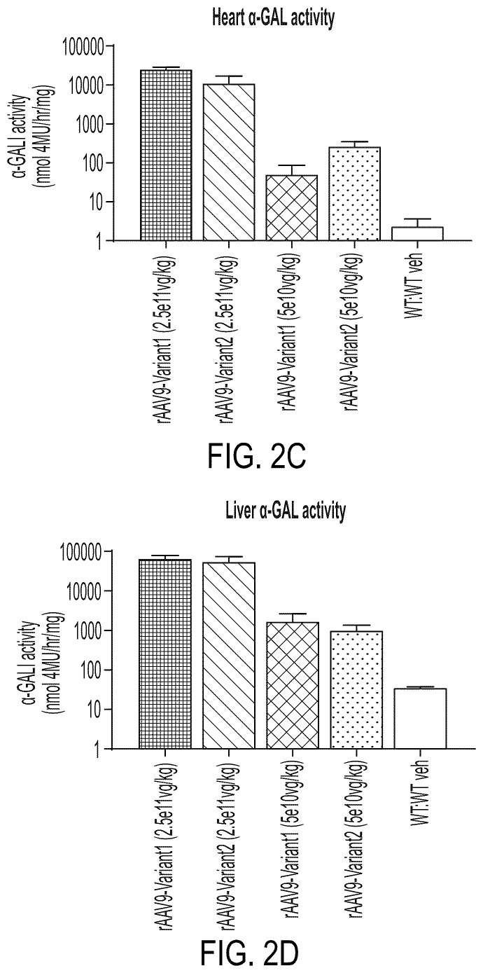

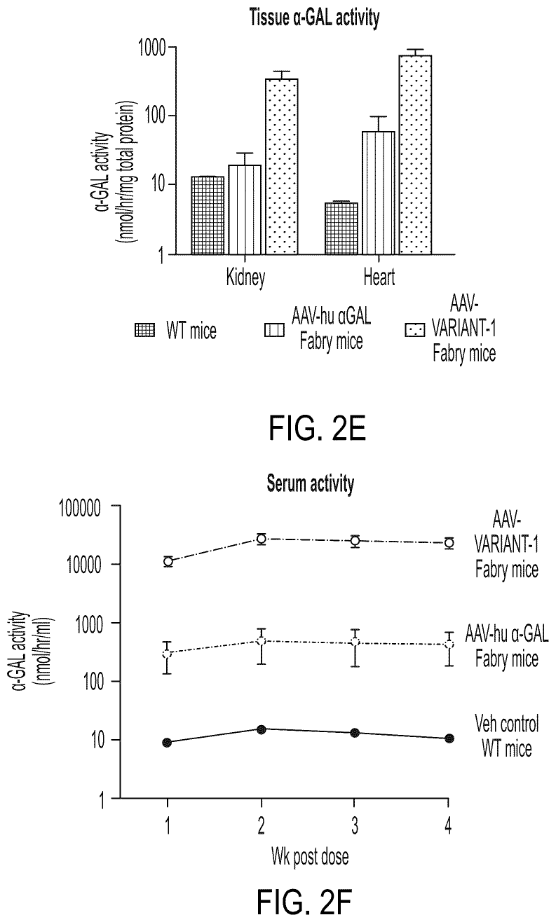

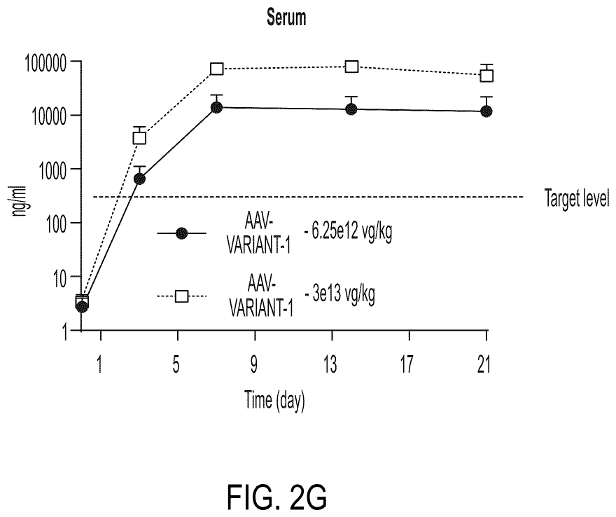

This example illustrates the sustained activity of α-GAL in kidney, heart and liver, in the same experiment discussed in Example 2, at 18weeks following IV administration of vectors as indicated in Table 4. A sustained elevation of α-GAL activity was also observed in serum throughout the study post vector administration at both doses (FIG. 2A). Greater than at least 6700-fold increase in alpha-galactosidase activity was seen in serum, compared to the vehicle.

Tissues from mice were homogenized in lysis buffer containing 10 mM HEPES with 0.5% Triton-X 100 and 1.5× Halt protease inhibitor cocktail, EDTA free, centrifuged and supernatant collected for analytical assays. Alpha galactosidase (α-GAL) activity in supernatant or serum was measured using a fluorescent substrate. Briefly, 2 ul of biological samples were incubated with 15 μL 4-MU-α-gal substrate solution (Research Products International Company, catalog #M65400) with α-galactosidase B inhibitor (N-acetyl-D-galactosamine, Sigma catalog #A-2795) at 37° C. for 60 minutes. The enzymatic reaction is stopped by addition of 200 μL glycine carbonate stop solution, pH 10.7. The 4-MU product is measured at the excitation wavelength 360 nm and emission wavelength 465 nm by a fluorescence plate reader. The concentrations of 4-MU in testing samples are calculated from the 4-MU calibration curve in the same plate. Tissue activity is normalized to total protein concentration determined by BCA assay (Thermo Scientific, catalog #23225).

The data from this Example demonstrated that both variant 1 and variant 2 produced sustained expression of α-GAL in kidney, heart and liver (FIG. 2B, 2C and 2D). The higher exposure in the liver indicates a higher amount of enzyme crosses the BBB to the nervous system (e.g., the PNS). Greater than 150-fold increase in α-galactosidase activity was seen in kidney, compared to the vehicle. Greater than 950-fold increase in α-galactosidase activity was seen in heart, compared to the vehicle.

Additionally, FIGS. 2E, compares the kidney and heart α-galactosidase activity of the Fabry mice transduced with 5e10 vg/kg of human wild-type α-galactosidase and Variant 1 respectively. Table 5 shows fold-increase of serum with wild-type human α-galactosidase and Variant 1 transductions.

TABLE 5
Fold increase in serum, kidney, and heart alpha
galactosidase activity in Fabry mice transduced
with wild-type alpha-galactosidase and Variant 1

| | AAV-hu | |
|---|---|---|
| Test article | α-GAL | AAV-Variant 1 |
| Serum α-GAL (×WT level) | >40× | >2200× |
| Kidney exposure | 1× | 27× |
| Heart exposure | 10× | 135× |

FIG. 2F shows elevation of α-GAL activity was also observed in serum in variant-1 and human alpha galactosidase transduced mice as compared to vehicle control. However, the alpha galactosidase activity is higher in mice transduced with variant 1 when compared to wild-type human alpha galactosidase.

FIG. 2G shows the serum alpha galactosidase concentrations in non-human primate (NHP), following single IV administration of Variant 1 at two different dosages −6.25e12 vg/kg and 3e13 vg/kg through 21 days. It was observed that in NHPs, α-GAL serum level reached >50-fold higher than predicted to be efficacious therapeutic levels even at the lowest tested dose of 6.25e12 vg/kg.

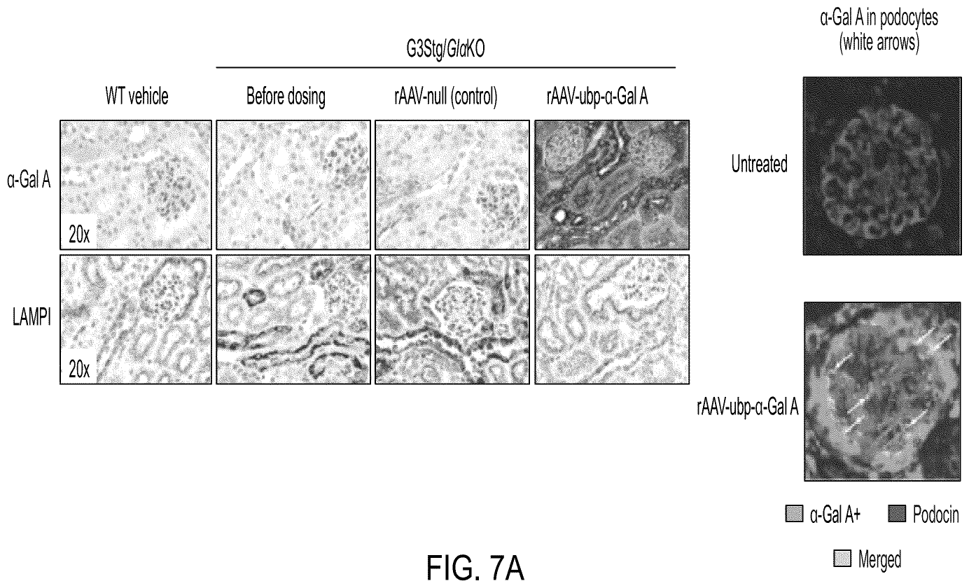

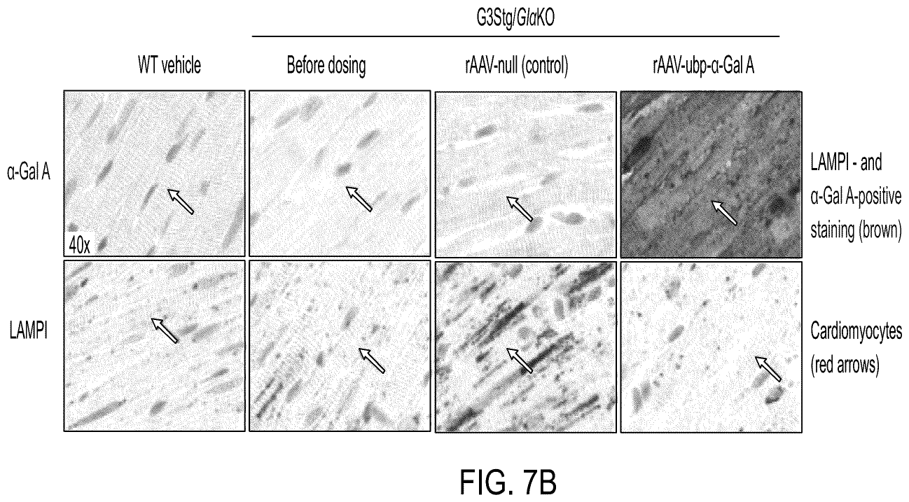

High levels of enzyme exposure were confirmed by immunohistochemistry in the kidney and the heart (FIGS. 7A and 7B). In addition, the α-GAL A enzyme exposure was also detected in cell types that are difficult to access, such as cardiomyocytes in the heart and podocytes in the kidneys (FIGS. 7A and 7B).

### Example 4. Substrate Levels Following Administration of Vectors

This Example examined the effects of Variant 1 and Variant 2 on GB3 and lysoGb3 reduction. It shows the GB3 and lysoGb3 substrate levels at 18 weeks following administration of vectors as discussed in Example 2.

G3Stg/GLAko mice have significantly higher substrate levels in various tissues. The levels of GB3 and lyso-Gb3 in the severe Fabry (G3Stg/GLAko) mice treated with the constructs as indicated in Table 4 were analyzed using mass-spectrometry. Substrates from serum and tissue samples were analyzed using an LC-MS method. Briefly substrate samples were extracted first using Chloroform: Methanol (v/v 2:1) and formic acid before running in HPLC and LC-MS/MS (Applied Biosystem API5000, Turbo Ion Spray Ionization, positive-ion mode).

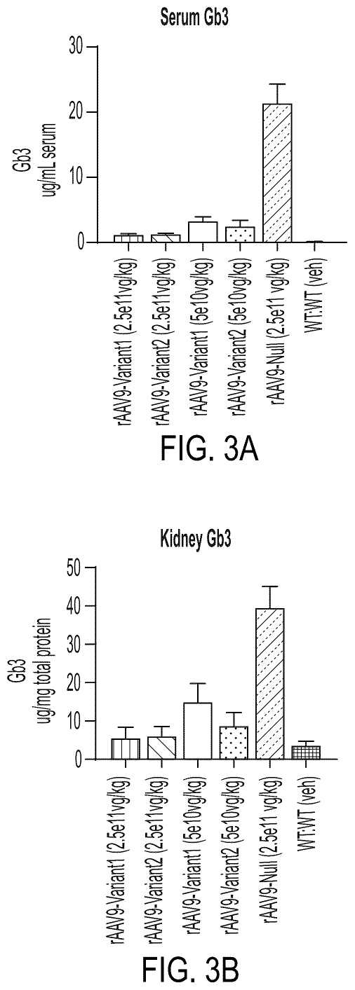

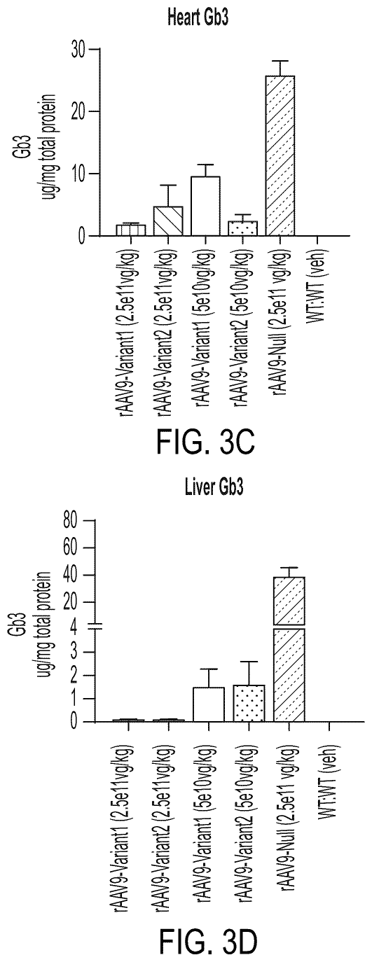

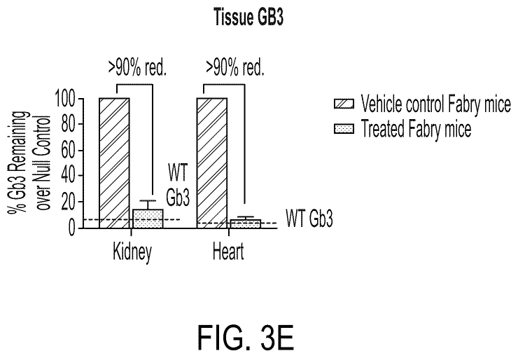

It was observed that both Variant 1 and Variant 2 can reduce GB3 accumulation associated with Fabry diseases in terminal serum, kidney, heart and liver (FIGS. 3A-3D). Greater than 90% reduction of Gb3 was observed in kidney and heart compared to vehicle control treated Fabry mice. Additionally, FIG. 3E compares the substrate reduction in kidney and heart of Fabry mice when transduced with wild-type human α-galactosidase or Variant 1. Table 6 shows fold decrease in kidney and heart of Fabry mice when transduced with wild-type human α-galactosidase or Variant 1.

TABLE 6
Fold decrease in kidney and heart of Fabry mice
when transduced with wild-type human alpha
galactosidase or Variant 1

| | AAV-hu | |
|---|---|---|
| Test article | α-GAL | AAV-Variant 1 |
| Kidney GB3 % reduction | 34% | 92% |
| Heart GB3 % reduction | 30% | 87% |

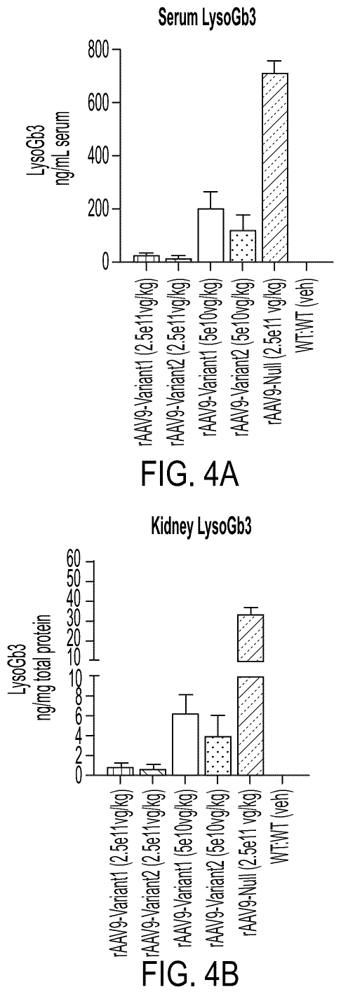

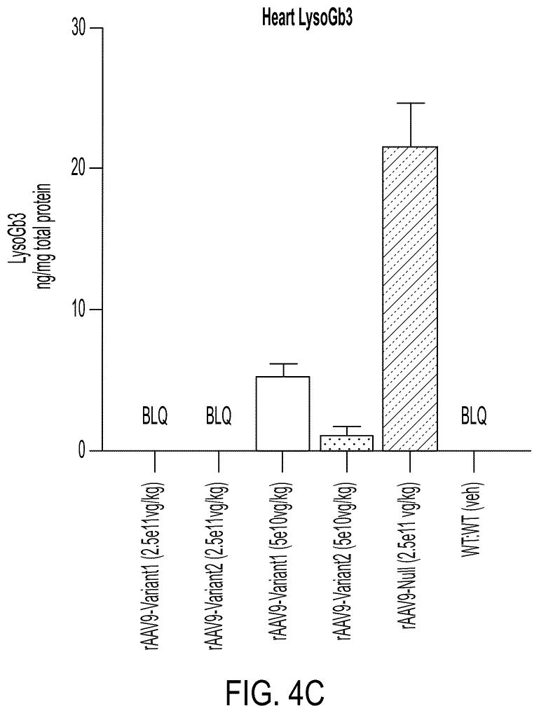

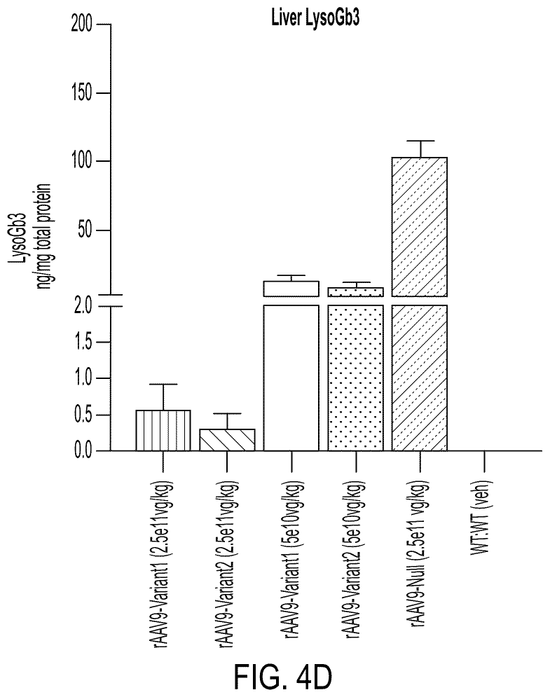

Similarly, reduced lysoGb3 accumulation was observed in serum, kidney, heart and liver after 18 weeks of administration (FIGS. 4A-4D). The substrate reduction in the multiple tissues indicates a systemic effect of the rAAV induced gene therapy. The treatment with Variant 1 or Variant 2 leads to complete or near-complete substrate normalization in Fabry mice.

### Example 5. Recovery of Thermal Sensitivity by Variant 1 and Variant 2 in Fabry Mouse Model (G3Stg/GLAko Mice)

This Example shows the effects of rAAV vectors expressing Variant 1 and Variant 2 on thermal sensitivity in G3Stg/GLAko mice.

In this study, the response of the mice to heat stimulus was tested using a hot-plate latency test. The hot plate test Eddy and Leimbach (1953) is a simple behavioral screen used for estimating the effects of NCEs on the threshold for detecting pain. It is based on the principle that when rodents are placed onto a hot surface they will initially demonstrate the aversive effects of the thermal stimulus by licking or flicking their paws and, ultimately, by overt attempts to escape the environment (jumping). Substances that alter nociceptive threshold either increase the latency to licking/jumping (analgesic effect) or decrease it (hyperalgesic effect). The hot-plate test is also a quick and relatively inexpensive way to assess acute, thermal pain, and an advantage over tail-flick/tail-withdrawal is the opportunity to test thermal sensitivity. This simple sensitivity test in mice is often used to indicate neuropathic pain (e.g., by heat) in human patients.

In the test, Mice from each group (Table 4), at pre-dose and at 2 weeks, 4 weeks, 8 weeks, 12 weeks, 16 weeks, and 18 weeks post-dose, were tested on the hot plate. The hot plate (Columbus Instruments) is preheated to 55° C. An open-ended cylindrical transparent plexiglas tube with a diameter of 30 cm is placed on top of the hot-plate to prevent mice from escaping but leaving the animals paws exposed to the hot plate. Using a stopwatch, the time from placing the mouse on the hot-plate to the time of the first paw-lick was measured. The latency to respond with a hind paw lick or shake/flutter, whichever came first, was measured to the nearest 0.1 second. Mice were removed automatically after 1 minute if no pain response is measured to prevent tissue damage.

Mice administered with Variant 1 and Variant 2 at the dose of 2.5e11 vg/kg recovered thermal sensitivity to a WT threshold (FIG. 5).

### Example 6. Amelioration of Neuropathy Associated with Fabry Disease in Mouse Model (G3Stg/GLAko Mice)

This Example examined the expression of neuropathy markers in a Fabry mouse model at 18 weeks after administration of vector.

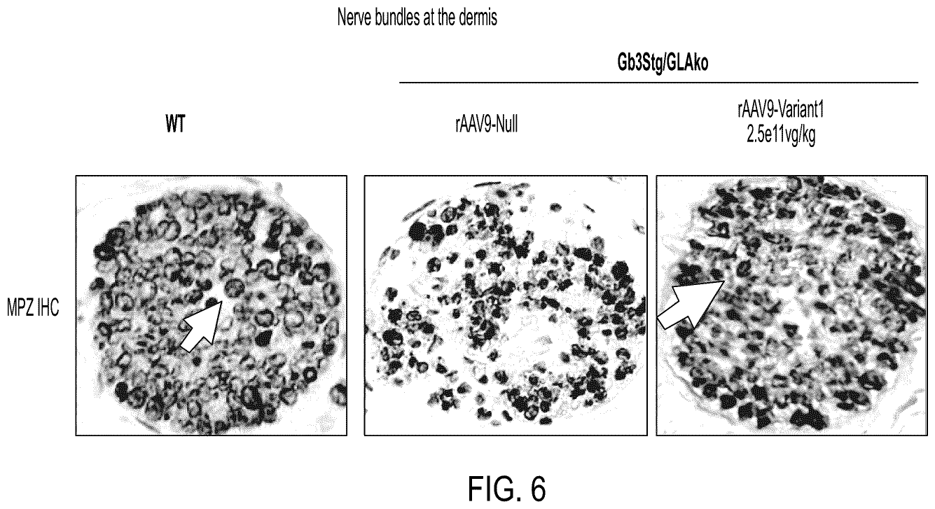

G3Stg/GLAko mice exhibit several signs of neuropathy as was observed in the histology of peripheral neurons after sacrifice. Footpads from hind paws from these animals were collected for analyses by immunohistochemistry to evaluate small fiber nerves to monitor any neuronal pathology in these animals. MPZ (myelin protein zero, the most abundant protein in myelin of nerves) staining (FIG. 6) reveals improved morphology and preserved density of dermis nerve bundles in G3Stg/GLAko mice treated with rAAV9-Variant 1, comparable to WT mice. As shown in FIG. 6, the intraepidermal nerve fiber density is preserved in G3Stg/GLAko mice treated with the rAAV9-α-GAL gene therapy vector, while the nerve density in G3Stg/GLAko mice treated with the control vector (rAAV-null) is not.

### Example 7. Nerve Structure and Lysosomal Burden in a Fabry Mouse Model

This Example examined the nerve structure and lysosomal burden in a Fabry mouse model at 18 weeks after administration of the rAAV9-α-GAL gene therapy vector.

The dorsal nerve root (DNR) samples were collected from G3Stg/GLAko mice treated with the rAAV9-α-GAL gene therapy vector. The samples from wild type mice and G3Stg/GLAko mice treated with a control vector (rAAV-null) were also collected. The tissues were fixed and processed for staining. Histological assessments were performed on fixed tissues subjected to immunohistochemistry or hematoxylin and eosin (HE) staining. Anti-LAMP1 antibodies were used for immunohistochemistry staining.

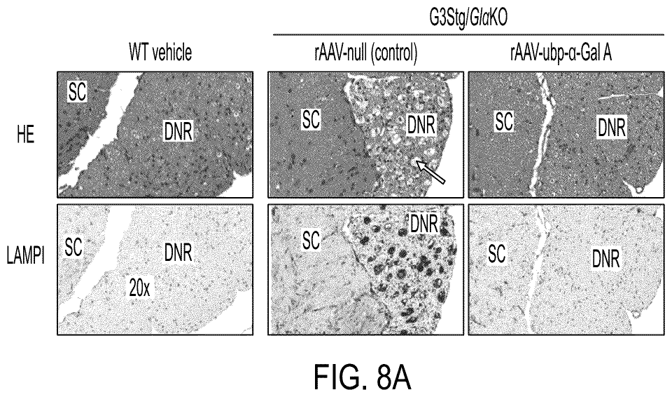

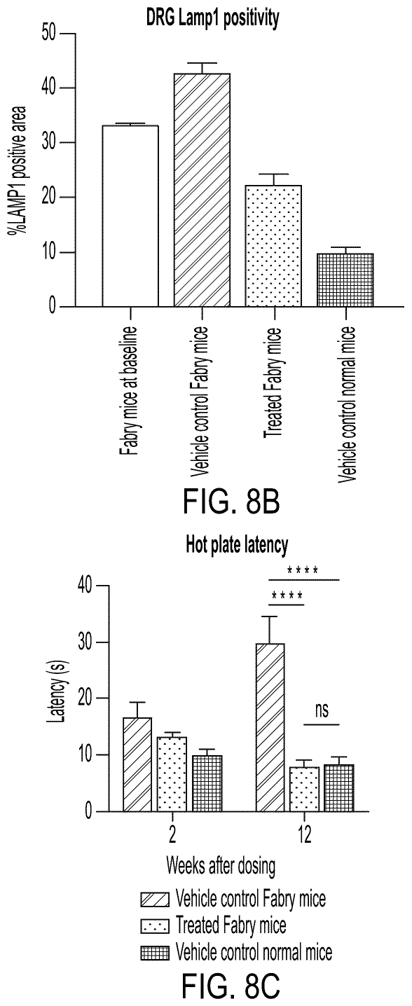

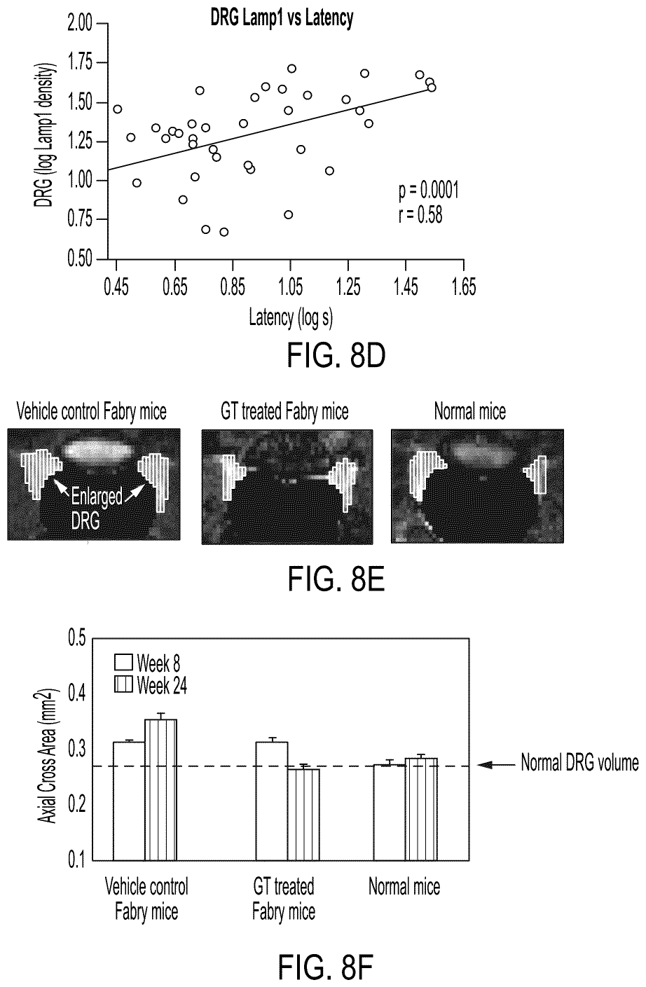

As shown in FIG. 8A, vacuoles were only observed in G3Stg/GLAko mice treated with the control vector. The treatment with the rAAV9-α-Gal gene therapy vector demonstrates reversal of the vacuolization in G3Stg/GLAko mice. LAMP1-positive staining was observed only in G3Stg/GLAko mice treated with the control vector, indicating reduced lysosomal burden. Substrate clearance resulted in multiple structural and functional improvements in treated mice, including normalization of lysosomal burden and prevention of vacuolization in dorsal root nerves.

Additionally FIG. 8B, 8C and 8D show reversal of peripheral nervous system (PNS) structural pathology translated into recovery and preservation of sensory deficit. FIG. 8B shows percentage of LAMP1 positive area in Fabry mice, as compared to Fabry mice treated with Variant 1 or Vehicle and normal mice. It was observed that Fabry mice treated with Variant 1 showed decrease in Lamp positive area compared to Fabry mice treated with vehicle. Additionally, the hot plate latency was observed at day 2 and day 12 in Fabry mice treated with Variant 1 and control (vehicle) and was compared to normal control (vehicle treated) mice (FIG. 8C). It was observed that the hot plate latency depicting sensory deficit in Fabry mice was restored with partial DRG substrate clearance. Furthermore, the DRG substrate burden, as evaluated using log of LAMP1 density, and hot plate latency showed correlation, while small fiber density did not show correlation with hot plate latency (FIG. 8D). This finding is believed to be consistent with reports on the mechanism of the PNS pathology in patients.

FIG. 8E is a DRG-MRI for mice to assess effect of gene therapy (GT) treatment on DRG volume in Fabry mice treated with vehicle and a tool gene therapy construct expressing a wild-type human α-galactosidase (GT), as compared to normal mice. FIG. 8F is a histogram of the area of DRG in gene therapy treated mice versus control (vehicle treated) mice or normal mice. It is observed that the gene therapy treated mice showed sustained decrease in DRG volume through week 24. It is anticipated that gene therapy will substantially improve the nerve physiology of human beings with Fabry disease. DRG volume is significantly increased in Fabry patients and is not affected by ERT treatment. Accordingly, DRG volume is a potential surrogate marker for monitoring treatment efficacy on Fabry PNS pathology.

### Example 8. Vacuolization and Lysosomal Burden in the Gastrointestinal Tract in a Fabry Mouse Model

This Example examined vacuolization and lysosomal burden in the gastrointestinal tract in a Fabry mouse model at 18 weeks after administration of the rAAV9-α-GAL gene therapy vector.

The smooth muscle tissue samples from the gastrointestinal tract were collected from G3Stg/GLAko mice treated with the rAAV9-α-GAL gene therapy vector. The samples from wild type mice, untreated G3Stg/GLAko mice and G3Stg/GLAko mice treated with a control vector (MY011) were also collected. The tissues were fixed and processed for staining. Histological assessments were performed on fixed tissues subjected to immunohistochemistry or hematoxylin and eosin (HE) staining. Anti-LAMP1 antibodies were used for immunohistochemistry staining.

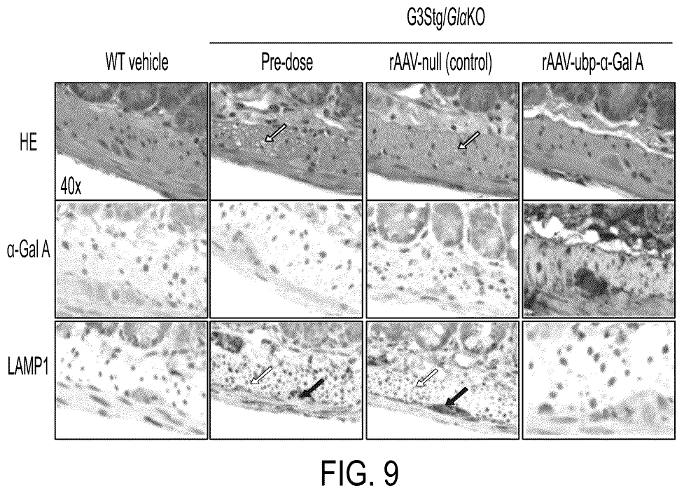

As shown in FIG. 9, the treatment with the rAAV-α-Gal gene therapy vector resulted in near-complete reversal of pathological vacuolization and significant reduction of lysosomal burden in the myenteric ganglion cells and transverse smooth muscles of the gastrointestinal tract. α-Gal A enzyme exposure was observed in mice treated with the rAAV9-α-Gal gene therapy vector. Vacuoles in the transverse smooth muscles of G3Stg/GlaKO duodenum were reversed after treating with the rAAV9-α-Gal gene therapy vector. LAMP1-positive staining in smooth muscles and myenteric ganglion cells was observed only in untreated and rAAV-null treated mice, but not in mice treated with the rAAV9-α-Gal gene therapy vector, suggesting reversal of lysosome burden in the gastrointestinal tract.

These immunohistochemistry results confirm high levels of enzyme exposure in different tissues and cells, including cardiomyocytes, podocytes, peripheral neurons and DRG. Together, these studies indicate the rAAV-α-Gal gene therapy vector can mediate the expression of an engineered α-GAL for halting further disease progression and even reversal of certain symptoms in a severe Fabry disease mouse model.

### Example 9. Comparison of Gene Therapy Using rAAV8-hα-GAL with Ubiquitous Promoter and Liver-Specific Promoter in GlaKO Mice

This Example evaluated the effects of gene therapy using rAAV8-hα-GAL (with liver-specific promoter) or rAAV9-hα-GAL (with ubiquitous promoter) vectors encoding wild-type alpha galactosidase.

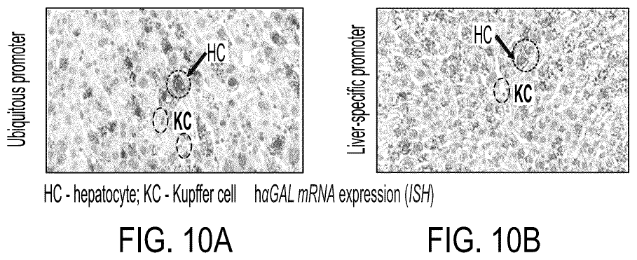

FIG. 10A and FIG. 10B show the immunohistochemistry results of GlaKO mice administered with a single IV dose of rAAV8-hα-GAL or rAAV9-hα-GAL with a liver specific promoter or ubiquitous promoter, encoding wildtype α-galactosidase at 2.5e11 vg/kg, in 12 weeks.

Table 7 summarizes the comparison between the ERT as compared to rAAV therapy in GlaKO mice.

TABLE 7
Comparison of ERT versus rAAV therapy

| | | rAAV-hα-GAL | rAAV-hα-GAL |
|---|---|---|---|
| | ERT | liver specific | ubiquitous |
| | | promoter | promoter |
| Serotype & | NA | rAAV with liver | rAAV with |
| promoter | | specific promoter | ubiquitous promoter |
| Dosing & | 1 mg/kg IV | 2.5E11 vg/kg IV | 2.5E11 vg/kg IV |
| Administration | injection | injection | injection |
| | 8× dose | | |
| Tissue Gb3 | Kidney: 53% | Kidney: 79% | Kidney: 87% |
| reduction | Heart: 73% | Heart: 98.5% | Heart: 99.5% |

It was observed that rAAV administration showed higher hα-GAL serum and tissue exposure, and better substrate clearance in Fabry mice. It was observed that Fabry mice (GlaKO mice) treated with AAV-hαGAL with both ubiquitous promoters and liver specific promoters showed better mRNA expression in the liver and will potentially provide better safety.

### Example 10. PK of Alpha Galactosidase Production and Substrate Reduction in Human Beings Using Translational Modelling

Target α-GAL levels are predicted to provide superior to ERT efficacy is achievable at a significantly low dose in human Fabry patients. This Example shows the translational modelling approach to show the alpha galactosidase production or Gb3 reduction in human beings as compared to ERT over 13 months.

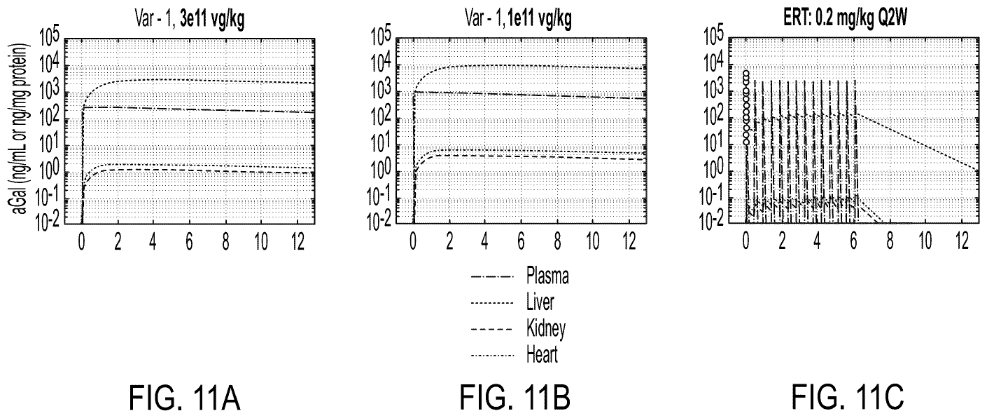

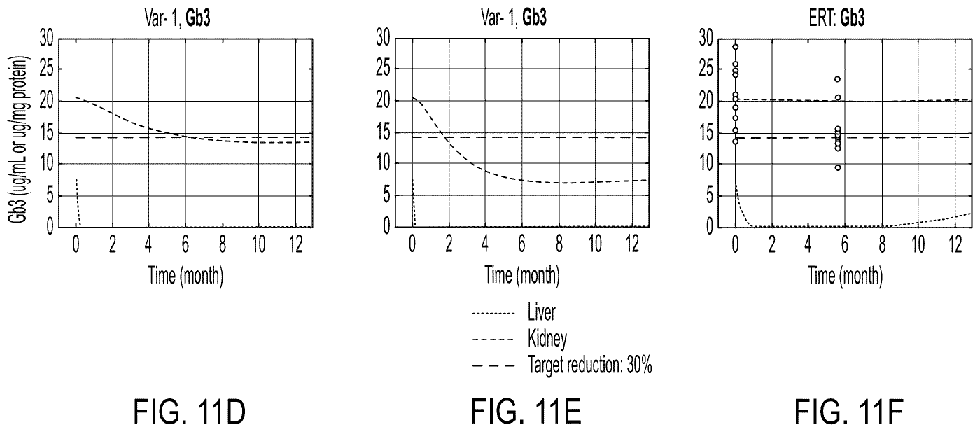

FIG. 11A-FIG. 11B show the alpha galactosidase production in various tissues projected at different dosages in 13 months. FIG. 11C shows the projected alpha galactosidase production in various tissues using ERT. FIG. 11D-FIG. 11E show the projected Gb3 reduction in various tissues at different dosages in 13 months. FIG. 11F shows the projected Gb3 reduction in various tissues using ERT. At least 30% Gb3 reduction in ERT resistant cell types at 6 months; could provide a meaningful change that could translate into functional improvements.

### Example 11. Minimal Efficacy Dose of Adeno-Associated Virus Vectors Expressing Human Alpha Galactosidase (α-GAL) Variants

The objective of this example was to evaluate the minimal effective dose of two recombinant adeno-associated virus vectors expressing human alpha galactosidase (α-GAL) variants when administered once intravenously (IV) in Fabry symptomatic mouse model (G3Stg/GLAko or HEMI;CAR) at 4 different doses and monitored for 4 weeks duration.

rAAV9-hα-GAL-variant 1 and rAAV9-hα-GAL-variant 2 were administered at 4 different doses: 2.5×10⁸, 2.5×10⁹, 2.5×10¹⁰, and 2.5×10¹¹ vg/kg. A null vector (rAAV9-null) was used as a control at 2.5×10¹¹ vg/kg did not produce circulated α-GAL activity or human protein detected.

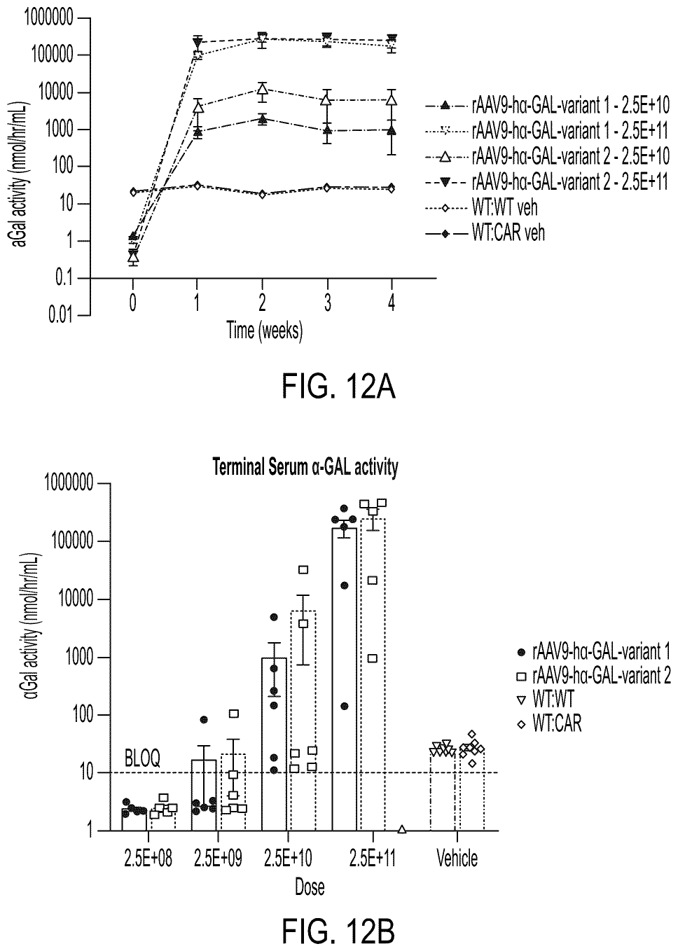

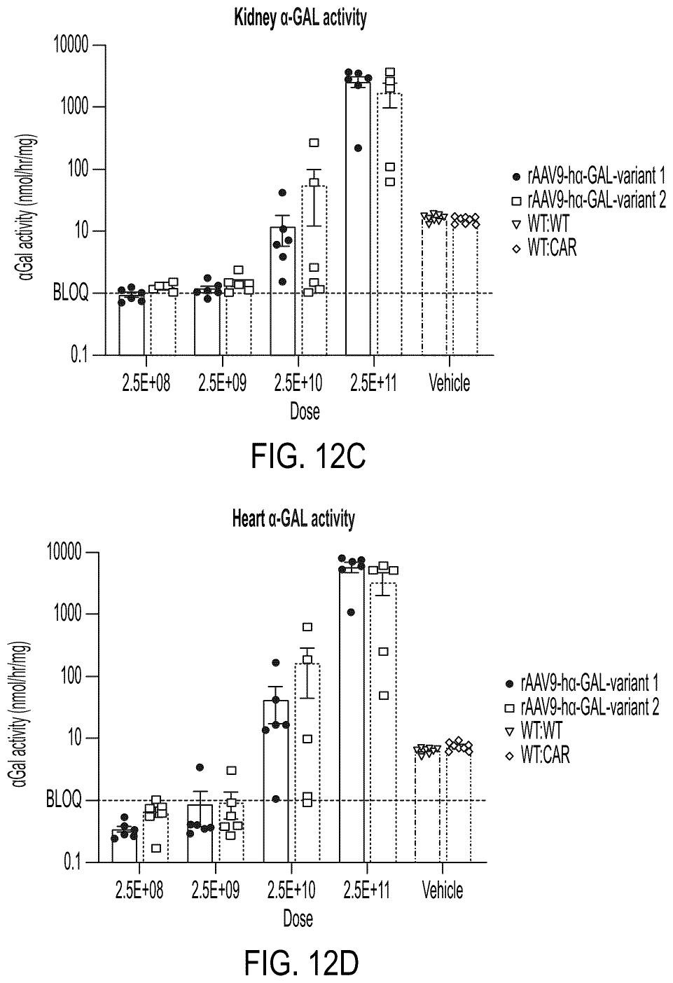

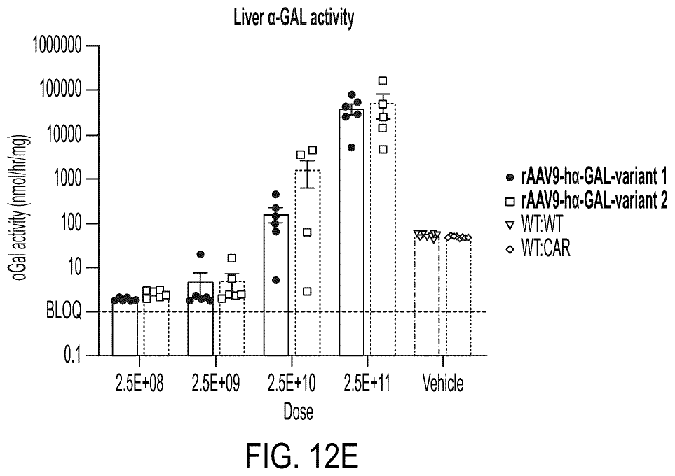

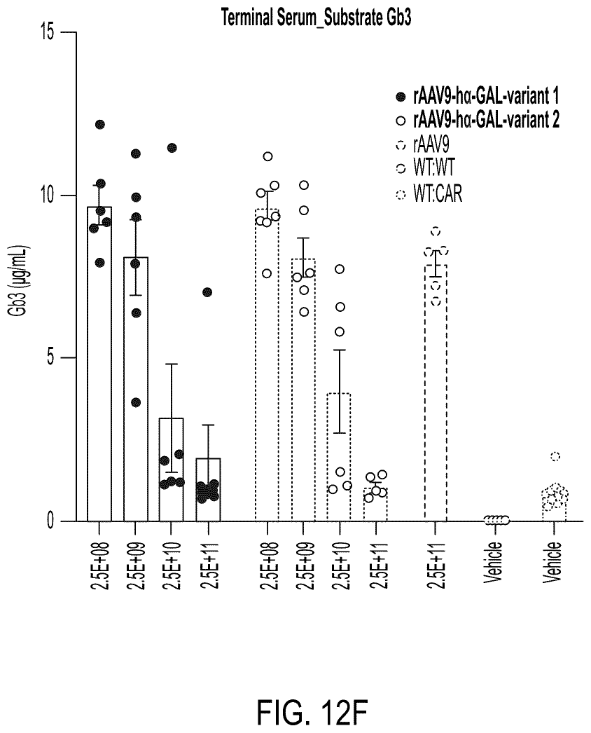

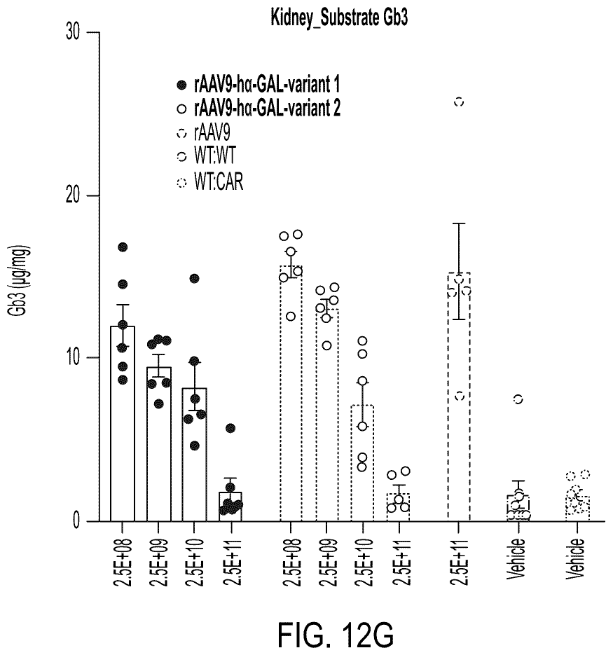

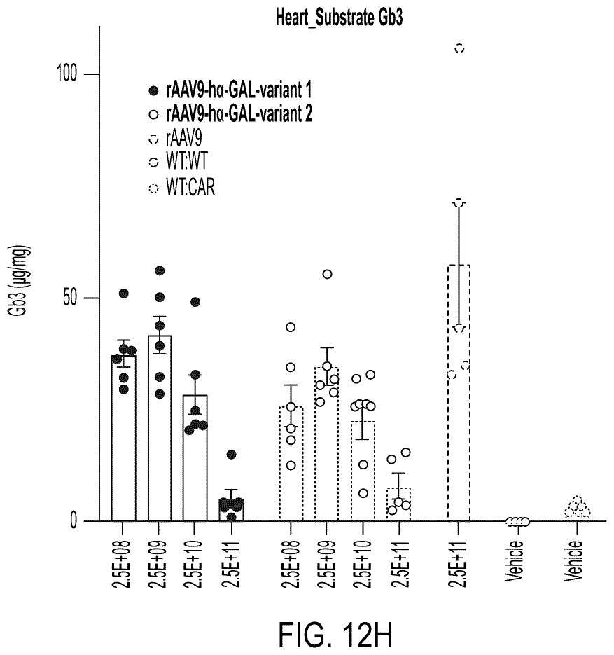

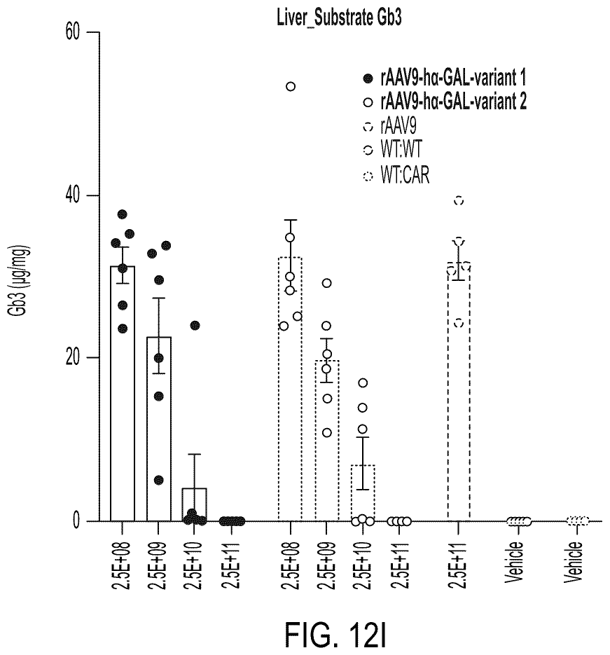

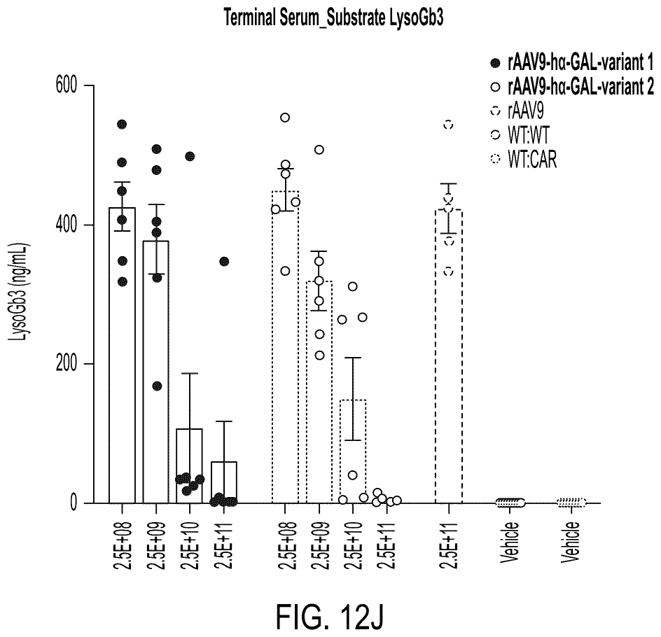

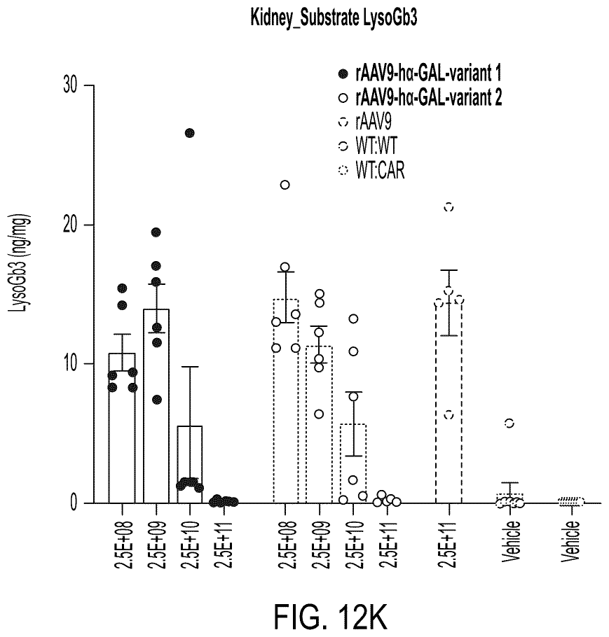

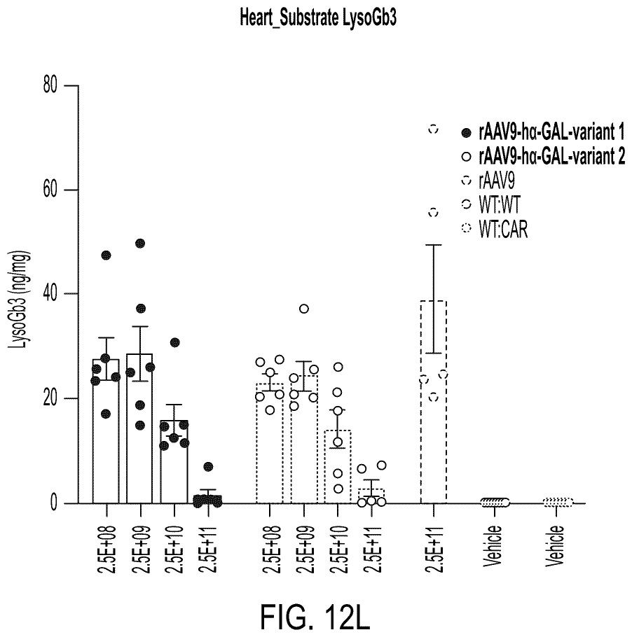

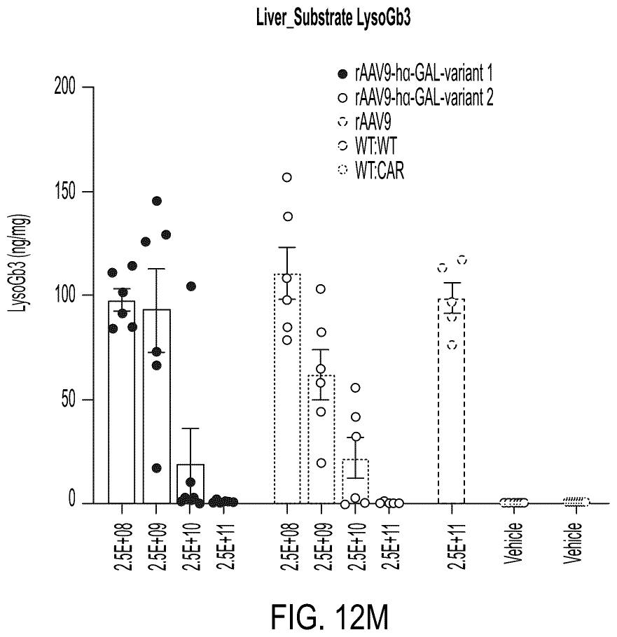

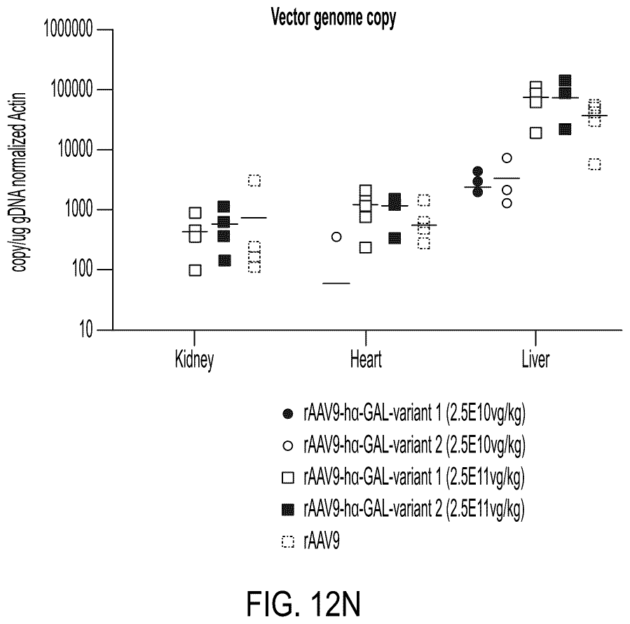

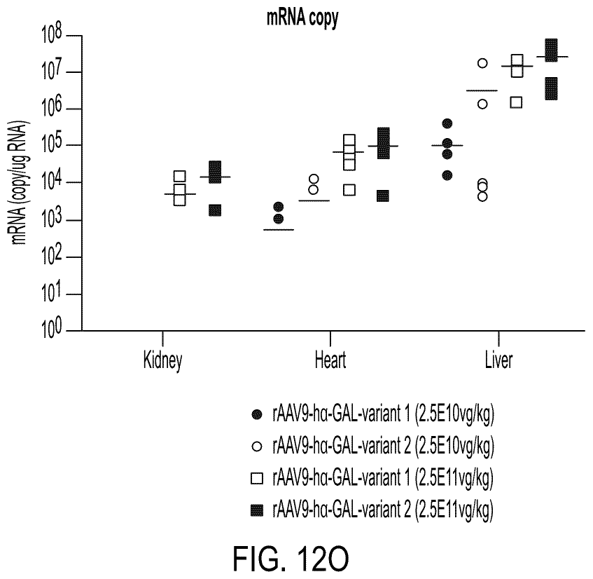

Mice treated with rAAV9-hα-GAL-variant 1 and rAAV9-hα-GAL-variant 2 showed dose-response increases in α-GAL activity in serum 1 week post injection and sustained through 4 weeks of study (FIG. 12A). Tables 8A and 8B show α-GAL activities in terminal serum and key tissues such as kidney, heart, and liver (also shown in FIG. 12B-FIG. 12E).

TABLE 8A
α-GAL Activity in serum and tissues

| | | Mean α-GAL activity | | | |
|---|---|---|---|---|---|
| | | (nmol/hr/mL of serum or mg protein) | | | |
| | Dose | Terminal | | | |
| Treatment | (vg/kg) | Serum | Kidney | Heart | Liver |
| Variant 1 | 2.5 × 10⁸ | 2.4 | 1.0 | 0.3 | 1.8 |
| Variant 1 | 2.5 × 10⁹ | 16 | 1.2 | 0.9 | 4.7 |
| Variant 1 | 2.5 × 10¹⁰ | 996 | 12 | 42 | 160 |
| Variant 1 | 2.5 × 10¹¹ | 174142 | 2563 | 5825 | 38990 |
| Variant 2 | 2.5 × 10⁸ | 2.1 | 1.3 | 0.6 | 2.5 |
| Variant 2 | 2.5 × 10⁹ | 21 | 1.5 | 0.9 | 5.1 |
| Variant 2 | 2.5 × 10¹⁰ | 6227 | 55 | 166 | 1986 |
| Variant 2 | 2.5 × 10¹¹ | 255928 | 1698 | 3318 | 51053 |
| rAAV9 | 2.5 × 10¹¹ | 0.0 | 0.9 | 0.6 | 3.4 |
| WT:WT | Vehicle | 25 | 16 | 6 | 51 |
| WT:CAR | Vehicle | 27 | 15 | 7 | 48 |

TABLE 8B
Fold α-GAL Activity in serum and tissues

| | | α-GAL Activity | | | |
|---|---|---|---|---|---|
| | | Fold α-GAL Activity | | | |
| | Dose | Terminal | | | |
| Treatment | (vg/kg) | Serum | Kidney | Heart | Liver |
| Variant 1 | 2.5 × 10⁸ | 0.0 | 0.1 | 0.1 | 0.0 |
| Variant 1 | 2.5 × 10⁹ | 1.0 | 0.1 | 0.1 | 0.1 |
| Variant 1 | 2.5 × 10¹⁰ | 40 | 0.7 | 6.8 | 3.1 |
| Variant 1 | 2.5 × 10¹¹ | 6938 | 159 | 932 | 760 |
| Variant 2 | 2.5 × 10⁸ | 0.0 | 0.1 | 0.1 | 0.0 |
| Variant 2 | 2.5 × 10⁹ | 1.0 | 0.1 | 0.1 | 0.1 |
| Variant 2 | 2.5 × 10¹⁰ | 248 | 3.4 | 26 | 39 |
| Variant 2 | 2.5 × 10¹¹ | 10196 | 106 | 531 | 995 |
| rAAV9 | 2.5 × 10¹¹ | 0.0 | 0.1 | 0.1 | 0.1 |
| WT:WT, | Vehicle | 1 | 1 | 1 | 1 |
| WT:CAR | | | | | |

High α-GAL activity was only detected at the top 2 doses of 2.5×10¹⁰ and 2.5×10¹¹ vg/kg. At 2.5×10¹¹ vg/kg, high α-GAL activity was achieved in terminal serum (more than 6,900- and 10,000-fold activity of WT) and in key target tissues (e.g., kidney >150- and >100-fold; heart >900- and >500-fold; liver >750- and ˜1,000-fold, respectively for rAAV9-hα-GAL-variant 1 and rAAV9-hα-GAL-variant 2; Table 8B) in a dose response manner.

Human α-GAL protein level was not detected in any samples of controls as expected. The protein level followed a dose-dependent increase in serum and evaluated tissues, reflecting the α-GAL activity each sample types and dose (Table 9). At lower 2 doses (2.5×10⁸, 2.5×10⁹ vg/kg) none of the variant test articles would be able to produce a detectable protein level in serum or tissues. Mice treated with rAAV9-hα-GAL-variant 2 at 2.5×10¹¹ vg/kg showed more α-GAL protein and activity in serum and liver than mice treated with rAAV9-hα-GAL-variant 1 at the same dose but resulted in less kidney and heart exposure, perhaps it's a results of different tissue uptake kinetics between 2 variants.

TABLE 9
α-GAL Concentration in serum and tissues

| | | α-GAL Concentration | | | |
|---|---|---|---|---|---|
| | | (ng/mL serum or mg of protein) | | | |
| | Dose | Terminal | | | |
| Treatment | (vg/kg) | Serum | Kidney | Heart | Liver |
| Variant 1 | 2.5 × 10⁸ | BLQ | BLQ | BLQ | BLQ |
| Variant 1 | 2.5 × 10⁹ | BLQ | BLQ | BLQ | BLQ |
| Variant 1 | 2.5 × 10¹⁰ | 295 | 26 | 36 | 188 |
| Variant 1 | 2.5 × 10¹¹ | 96673 | 3108 | 10884 | 51242 |
| Variant 2 | 2.5 × 10⁸ | BLQ | BLQ | BLQ | BLQ |
| Variant 2 | 2.5 × 10⁹ | BLQ | BLQ | BLQ | BLQ |
| Variant 2 | 2.5 × 10¹⁰ | 878 | 23 | 100 | 353 |
| Variant 2 | 2.5 × 10¹¹ | 147238 | 1636 | 6531 | 85132 |
| rAAV9 | 2.5 × 10¹¹ | BLQ | BLQ | BLQ | BLQ |
| WT:WT | Vehicle | BLQ | BLQ | BLQ | BLQ |
| WT:CAR | Vehicle | BLQ | BLQ | BLQ | BLQ |

α-GAL = alpha-galactosidase A; GLAKO = α-GAL-deficient.
Values are average α-GAL concentration (ng/mL serum or mg of tt protein).
BLQ = Below Limit of Quantification

### Gb3 and lysoGb3

Both Gb3 and lysoGb3 substrates accumulated in diseased control injected with a Null vector rAAV9 at 2.533 10¹¹ vg/kg compared to WT:WT control in terminal serum and key tissues. WT:CAR, mice that have a copy of G3S gene knock-in, showed more Gb3 substrates especially in heart and serum more than WT: WT but less than G3Stg/GLAko (Table 10, FIG. 12F-FIG. 12M).

Both Gb3 substrates were reduced in terminal serum and target tissues in a dose dependent manner when mice were treated with either rAAV9-Variant 1 or rAAV9-Variant 2, reaching near normal level at the highest dose of 2.5×10¹¹ vg/kg. This translated to >85% of tissue Gb3 reduction from the G3Stg/GLAko Null control. Near normalized of Gb3 substrates in liver (>75%) and terminal serum (>) in mice treated with 2.5×10¹⁰ vg/kg, but less efficacy in kidney and heart. At 2.5×10¹⁰ vg/kg dose, rAAV9-Variant 2 has a slightly better Gb3 reduction in kidney and heart (53%, 61% respectively) than rAAV9-Variant 1 (46%, 51% respectively).

TABLE 10
Percent Gb3 substrate reduction

| | | Gb3 Substrate Reduction | | | |
|---|---|---|---|---|---|
| | Dose | Terminal | | | |
| Treatment | (vg/kg) | Serum | Kidney | Heart | Liver |
| Variant 1 | 2.5 × 10⁸ | −23% | 21% | 35% | 2% |
| Variant 1 | 2.5 × 10⁹ | −2% | 38% | 28% | 29% |
| Variant 1 | 2.5 × 10¹⁰ | 60% | 46% | 51% | 87% |
| Variant 1 | 2.5 × 10¹¹ | 76% | 88% | 91% | 100% |
| Variant 2 | 2.5 × 10⁸ | −22% | −3% | 55% | −2% |
| Variant 2 | 2.5 × 10⁹ | −3% | 15% | 40% | 38% |
| Variant 2 | 2.5 × 10¹⁰ | 50% | 53% | 61% | 78% |
| Variant 2 | 2.5 × 10¹¹ | 87% | 88% | 86% | 100% |
| rAAV9 | 2.5 × 10¹¹ | 0% | 0% | 0% | 0% |

Gb3 = globotriaosylceramide-3; GLAKO = α-GAL-deficient.
Values are average % reduction over null control (rAAV9).

A similar dose-dependent trend of lysoGb3 substrates reduction was observed (Table 11 and FIG. 12J-FIG. 12M).

TABLE 11
Percent lysoGb3 substrate reduction

| | | LysoGb3 Substrate Reduction | | | |
|---|---|---|---|---|---|
| | Dose | Terminal | | | |
| Treatment | (vg/kg) | Serum | Kidney | Heart | Liver |
| Variant 1 | 2.5 × 10⁸ | −1% | 25% | 29% | 1% |
| Variant 1 | 2.5 × 10⁹ | 10% | 3% | 27% | 6% |
| Variant 1 | 2.5 × 10¹⁰ | 74% | 61% | 59% | 81% |
| Variant 1 | 2.5 × 10¹¹ | 86% | 99% | 96% | 99% |
| Variant 2 | 2.5 × 10⁸ | −6% | −3% | 41% | −12% |
| Variant 2 | 2.5 × 10⁹ | 24% | 21% | 38% | 37% |
| Variant 2 | 2.5 × 10¹⁰ | 65% | 60% | 64% | 78% |
| Variant 2 | 2.5 × 10¹¹ | 99% | 99% | 93% | 100% |
| rAAV9 | 2.5 × 10¹¹ | 0% | 0% | 0% | 0% |

Lyso Gb3 = lyso-globotriaosylceramide-3; GLAKO = α-GAL-deficient.
Values are average % reduction over null control (rAAV9−).

### Vector Genome and mRNA Copy Number

Vector genome (vg) and mRNA copy number were determined from kidney, heart and liver tissue. WT:WT and WT:CAR controls did not detect any vector genome copy as expected. The highest dose of 2.5×10¹¹ vg/kg treatment of all tissues resulted in detectable vg copy number and translated into a similar result for mRNA copy number (FIG. 12N-FIG. 12O). At a lower dose of 2.5×10¹⁰ vg/kg, only liver vg and mRNA can be detected.

It was observed that only top 2 doses (2.5×10¹⁰ and 2.5×10¹¹ vg/kg) showed significant exposure and substrate reduction efficacy in serum and evaluated tissues in a dose-dependent response.

### Example 12. Minimal Efficacy Dose of Adeno-Associated Virus Vectors Expressing Human Alpha Galactosidase (α-GAL) Variants

The objective of this example is to evaluate rAAV9-Variant 1 durability in GlaKO male mice for 8 months, with an interim sacrifice at 3 months post injection at 2 different doses (1e11 and 2.5e10 vg/kg). rAAV9-null was used as a negative control at the highest dose, 1e11 vg/kg, only.

GlaKO and WT control animals were injected with Variant 1 at 2 different doses (1e11 and 2.5e10 vg/kg) or injected with rAAV9 null control at the highest dose (1e11 vg/kg) through IV route of administration and evaluated for serum and tissue exposure, substrate reduction, urinary and blood chemistries, histopathological evaluation, and Echocardiogram.

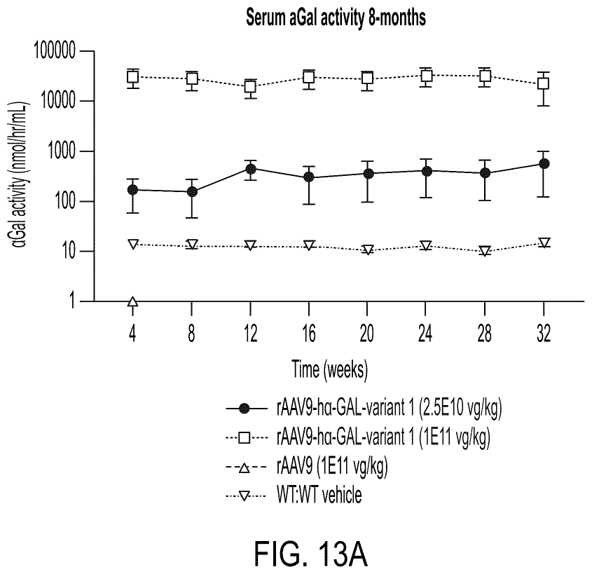

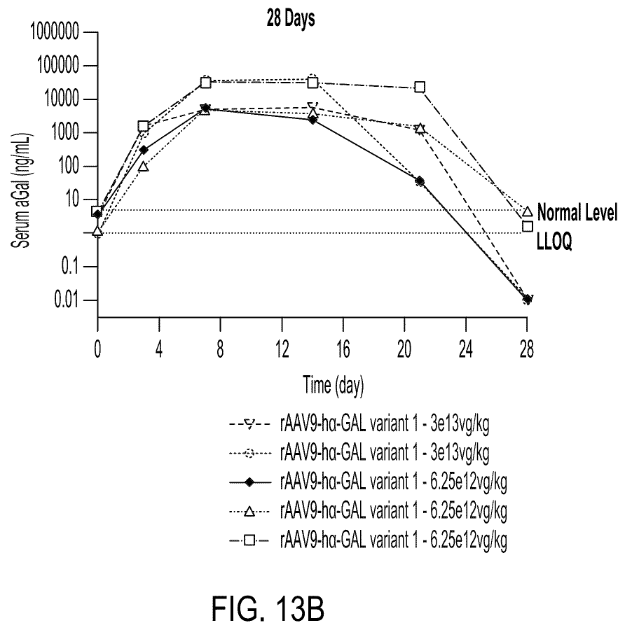

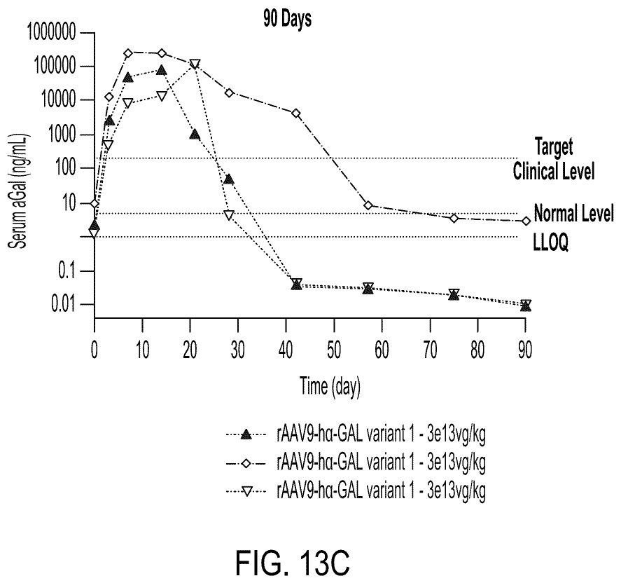

Data generated from this study would be serum and tissue α-GAL activity from during 8-months (Table 12) cohort. Serum α-GAL activity throughout 8 months were plotted in FIG. 13A.

TABLE 12
Serum and tissue alpha-Gal activity from 8-months coho|rt

| | | | Serum Activity (nmol/hr/mL serum) | | | | | | | | | | |
|---|---|---|---|---|---|---|---|---|---|---|---|---|---|
| Mouse | | | Week | Week | Week | Week | Week | Week | Week | Week | Tissue (nmol/hr/mg total protein) | | |
| strain | Treatment | Mouse | 4 | 8 | 12 | 16 | 20 | 24 | 28 | 32 | Liver | Kidney | Heart |
| WT:WT | Vehicle | E1 | 18.6 | 12.5 | 20.9 | 16.5 | 13.4 | 13.1 | 14.8 | 17.8 | 27.6 | 13.1 | No |
| | | | | | | | | | | | | | sample |
| | | E2 | 13.0 | 13.4 | No | No | No | No | No | No | No | No | No |
| | | | | | sample | sample | sample | sample | sample | sample | sample | sample | sample |
| | | E3 | 9.8 | 11.8 | 8.1 | 10.8 | 7.9 | 10.0 | No | No | No | No | No |
| | | | | | | | | | sample | sample | sample | sample | sample |
| | | E4 | 11.0 | 9.1 | 10.9 | 11.6 | 9.1 | 15.5 | 9.1 | 20.8 | 35.6 | 12.6 | No |
| | | | | | | | | | | | | | sample |
| | | E5 | 13.4 | 9.8 | 13.9 | 12.0 | 8.4 | 13.4 | 14.3 | 11.2 | 36.2 | 13.3 | BQL |
| | | E6 | 12.0 | 11.5 | 10.0 | 11.8 | 7.9 | 12.9 | 7.3 | 19.5 | 24.2 | 13.6 | BQL |
| | | E7 | 14.1 | 9.2 | 11.5 | 9.6 | 9.6 | 15.6 | 8.7 | 12.75 | 28.5 | 13.2 | BQL |
| | | E8 | 8.7 | 11.5 | 15.1 | 14.5 | 10.9 | 13.2 | 10.2 | 12.61 | 30.4 | BQL | BQL |
| | | E9 | 10.8 | 13.2 | 13.4 | 12.8 | 13.1 | 8.9 | 7.0 | 7.41 | 27.4 | 11.3 | BQL |
| | | E10 | 28.5 | 28.0 | 12.2 | 13.8 | 15.0 | 10.1 | 7.9 | No | No | No | No |
| | | | | | | | | | | sample | sample | sample | sample |
| GLA KO | rAAV9 | F1 | BQL | BQL | BQL | No | No | No | No | No | No | No | No |
| | | | | | | sample | sample | sample | sample | sample | sample | sample | sample |
| | | F2 | BQL | BQL | BQL | BQL | BQL | BQL | BQL | BQL | BQL | BQL | No |
| | | | | | | | | | | | | | sample |
| | | F3 | BQL | BQL | BQL | BQL | BQL | BQL | BQL | BQL | BQL | BQL | No |
| | | | | | | | | | | | | | sample |
| | | F4 | BQL | BQL | BQL | BQL | BQL | BQL | BQL | BQL | BQL | BQL | No |
| | | | | | | | | | | | | | sample |
| | | F5 | BQL | BQL | BQL | BQL | BQL | BQL | BQL | BQL | BQL | BQL | BQL |
| | | F6 | BQL | BQL | BQL | BQL | BQL | BQL | BQL | BQL | BQL | BQL | BQL |
| | | F7 | BQL | BQL | BQL | BQL | BQL | BQL | BQL | BQL | BQL | BQL | BQL |
| | | F8 | BQL | BQL | BQL | BQL | BQL | BQL | BQL | BQL | No | No | No |
| | | | | | | | | | | | sample | sample | sample |
| | | F9 | BQL | BQL | BQL | BQL | BQL | BQL | BQL | No | BQL | BQL | BQL |
| | | | | | | | | | | sample | | | |
| | | F10 | BQL | BQL | BQL | No | No | No | No | No | No | No | No |
| | | | | | | sample | sample | sample | sample | sample | sample | sample | sample |
| | rAAV9-hα- | G1 | 1129.5 | 2080.8 | 754.4 | 366.6 | 364.2 | 345.8 | 181.1 | 95.0 | 54.7 | BQL | No |
| | GAL-variant | | | | | | | | | | | | sample |
| | 1 | G2 | 286.5 | 234.9 | 51.5 | 84.7 | 99.6 | 210.7 | 111.3 | 248.8 | 140.9 | BQL | No |
| | 1.00E+11 | | | | | | | | | | | | sample |
| | | G3 | BQL | BQL | BQL | BQL | BQL | BQL | BQL | BQL | BQL | BQL | No |
| | | | | | | | | | | | | | sample |
| | | G4 | 83118.9 | 87297.7 | 50266.2 | 67341.2 | 68703.4 | 69424.4 | 72554.2 | 113478.7 | 7932.8 | 6055.8 | No |
| | | | | | | | | | | | | | sample |
| | | G5 | BQL | BQL | BQL | BQL | BQL | BQL | BQL | BQL | BQL | BQL | 36.3 |
| | | G6 | 10229.2 | 9165.6 | 9801.8 | 13050.4 | 8937.1 | 6752.6 | 6156.0 | 3677.7 | 1106.7 | 36.8 | 159.0 |
| | | G7 | 16603.6 | 13657.0 | 8354.3 | 19717.4 | 21988.2 | 22041.2 | 21964.3 | 34.8 | 4549.6 | 547.7 | 876.8 |
| | | G8 | 51960.4 | 54341.0 | 31727.1 | 42268.9 | 43257.1 | 37998.3 | 44671.4 | 57621.8 | 16689.6 | 1189.8 | 7045.2 |
| | | G9 | 680.3 | 1780.9 | 1595.1 | 223.2 | 140.5 | BQL | BQL | 3873.6 | BQL | BQL | 107.2 |
| | | G10 | 88179.9 | 62330.3 | 55927.7 | 94082.1 | 83395.1 | 99482.4 | 85855.5 | 5007.2 | 31173.7 | 1590.0 | 9431.3 |
| | rAAV9-hα- | H1 | 10.5 | 14.4 | 892.1 | BQL | BQL | BQL | BQL | BQL | BQL | BQL | No |
| | GAL-variant | | | | | | | | | | | | Sample |
| | 1 | H2 | 88.0 | 15.8 | BQL | BQL | BQL | BQL | BQL | BQL | BQL | BQL | No |
| | 2.50E+10 | | | | | | | | | | | | sample |
| | | H3 | 16.6 | 15.4 | BQL | BQL | BQL | BQL | BQL | BQL | BQL | BQL | No |
| | | | | | | | | | | | | | sample |
| | | H4 | 34.4 | 17.9 | 143.7 | BQL | BQL | BQL | BQL | BQL | BQL | BQL | No |
| | | | | | | | | | | | | | sample |
| | | H5 | 244.4 | 230.3 | 185.7 | 184.5 | 207.9 | 192.9 | 166.1 | 219.1 | 19.3 | BQL | 14.0 |
| | | H6 | 84.4 | 73.6 | 95.7 | 62.3 | BQL | 57.2 | 42.8 | 36.9 | BQL | BQL | 19.5 |
| | | H7 | 1150.7 | 1158.3 | 959.1 | 932.3 | 878.4 | 966.3 | 924.4 | 1413.4 | 50.7 | BQL | 16.0 |
| | | H8 | 38.3 | 32.4 | BQL | BQL | BQL | BQL | BQL | BQL | BQL | BQL | 15.1 |
| | | H9 | 14.5 | 15.3 | BQL | BQL | BQL | BQL | BQL | BQL | BQL | BQL | 16.2 |
| | | H10 | 17.1 | 14.9 | BQL | 24.4 | BQL | BQL | BQL | BQL | BQL | BQL | 18.0 |

Sustained α-GAL activity was observed in liver, kidney and heart throughout the test duration at both tested concentrations.

The recombinant adenovirus rAAV9-hα-GAL variant 1 was administered intravenously to non-human primates. It was challenging to source AAV9 seronegative animals. High transgene expression was observed in most treated animals despite high pre-existing Nabs (FIG. 13B, FIG. 13C). FIGS. 13B and FIG. 13C show serum alpha galactosidase concentrations over 28-90 days at different concentrations.

The results indicate that he level of α-Gal expression was lower in NHP than in mice but still reached >3000× of normal and >50× of target clinical level with a low dose of 6.25e12 vg/kg. The results in NHP support low rAAV9-hα-GAL variant 1 therapeutic dose within e12 vg range.

### Equivalents and Scope

Those skilled in the art will recognize or be able to ascertain using no more than routine experimentation, many equivalents to the specific embodiments of the invention described herein. The scope of the present invention is not intended to be limited to the above description, but rather is as set forth in the following claims:

1. A method of alleviating or ameliorating peripheral neuropathy in a subject diagnosed with Fabry disease comprising administering to the subject a composition comprising a recombinant adeno-associated viral vector (rAAV) that comprises a polynucleotide encoding α-GAL enzyme or a variant thereof, wherein the subject diagnosed with Fabry disease has or is developing one or more neuropathy associated symptoms.

2. The method of claim 1, wherein the peripheral neuropathy manifests as neuropathic pain, thermal hypoesthesia, hearing loss, other sensory deficiencies and/or gastrointestinal disturbances.

3. The method of claim 2, wherein the neuropathic pain is alleviated.

4. The method of claim 2, wherein the thermal hypoesthesia is alleviated.

5. A method of improving peripheral abnormalities in nerve fibers in a subject with Fabry disease comprising administering to the subject a composition comprising a recombinant adeno-associated viral vector (rAAV) that comprises a polynucleotide encoding α-GAL enzyme or a variant thereof.

6. The method of claim 5, wherein the nerve fibers are small nerve fibers.

7. The method of claim 6, wherein the improvement includes the preservation of small myelinated nerve fibers.

8. The method of claim 6, wherein the improvement includes the preservation of small unmyelinated nerve fibers.

9. The method of any one of claims 5-8, wherein the density of sensory nerve fibers is preserved.

10. The method of any one of the preceding claims, wherein the administration of the rAAV decreases the accumulation of globotriaosylceramide (GB3) in the peripheral nervous system as evidenced by reduction in LAMP1 levels in the DRG.

11. A method for reducing the accumulation of globotriaosylceramide (GB3) in the peripheral nervous system caused by α-galactosidase A deficiency comprising administering to a subject in need a composition comprising a recombinant adeno-associated viral vector (rAAV) that comprises a polynucleotide encoding α-GAL enzyme or a variant thereof.

12. The method of claim 11, wherein the accumulation of in the Dorsal root ganglion (DRG) is reduced as evidenced by reduction in LAMP1 levels in the DRG.

13. The method of claim 11, wherein the accumulation of in peripheral nerves is decreased as evidenced by reduction in LAMP1 levels in the nerves.

14. A method for treating neuropathy in a subject with Fabry disease, comprising
(a) determining the subject has or is developing one or more neuropathy associated symptoms;
(b) administering to the subject a therapeutically effective amount of a composition comprising a recombinant adeno-associated viral vector (rAAV) that comprises a polynucleotide encoding α-GAL enzyme or a variant thereof; and
(c) assessing said one or more neuropathy associated symptoms in the subject after the treatment.

15. The method of claim 14, wherein the one or more neuropathy associated symptoms include chronic burning pain, attacks of excruciating pain, paresthesias/dysesthesias, sensory loss, hypohidrosis/anhidrosis, Abdominal cramp, (post-prandial) diarrhea, bloating, nausea, and Tinnitus, and hearing loss.

16. The method of claim 14 or 15, wherein the assessment is performed 2 weeks, 5 weeks, 10 weeks, 18 weeks, or 6 months after the administration of the composition.

17. The method of any one of the preceding claims, wherein the rAAV vector is a rAAV vector packaged in a capsid with broad tissue tropism, the vector comprising:
a. a 5′ inverted terminal repeat (ITR);
b. a ubiquitous promoter;
c. the polynucleotide encoding α-GAL enzyme;
d. a poly A; and
e. a 3′ ITR.

18. The method of any one of claims 1-16, wherein the rAAV vector is a rAAV vector packaged in a capsid with broad tissue tropism, the vector comprising:
a. a 5′ inverted terminal repeat (ITR);
b. a liver specific promoter;
c. the polynucleotide encoding α-GAL enzyme;
d. a poly A; and
e. a 3′ ITR.

19. The method of any one of claims 1-16, wherein the rAAV vector is a rAAV vector packaged in a capsid with tropism specific to the nervous system, the vector comprising:
a. a 5′ inverted terminal repeat (ITR);
b. a ubiquitous promoter;
c. the polynucleotide encoding α-GAL enzyme;
d. a poly A; and
e. a 3′ ITR.

20. The method of any one of claims 17-19, wherein the vector further comprises a woodchuck hepatitis virus posttranscriptional regulatory element (WPRE).

21. The method of any one of claims 17-20, wherein the polynucleotide encodes α-GAL enzyme comprising an amino acid sequence selected from SEQ ID Nos: 2, 3, 6 and 7

22. The method of any one of claims 17-21, wherein the polynucleotide encoding α-GAL enzyme comprises a nucleotide sequence of SEQ ID Nos: 9, 10, 12 and 13.

23. The method of any one of claims 17-22, wherein the AAV capsid is a wide-tropism AAV capsid selected from an AAV1 capsid, AAV2 capsid, AAV3 capsid, AAV4 capsid, AAV5 capsid, AAV6 capsid, AAV7 capsid, AAV8 capsid, or AAV9 capsid.

24. The method of claim 23, wherein the wide-tropism AAV capsid is AAV9 capsid.

25. The method of claim 24, wherein the AAV9 capsid is naturally occurring or engineered.

26. The method of any one of claims 17-25, wherein the ubiquitous promoter comprises a chicken β actin (CBA) promoter, a EF-1α promoter, a PGK promoter, a UBC promoter, a LSE beta-glucuronidase (GUSB) promoter, a ubiquitous chromatin opening element (UCOE) promoter, a cyto-megalo-virus (CMV) enhancer, a chicken beta actin promoter, or one or more introns.

27. The method of claim 26, wherein the ubiquitous promoter comprising a cyto-megalo-virus (CMV) enhancer, chicken beta actin promoter, and a rabbit beta globin intron.

28. The method of claim 26, wherein the ubiquitous promoter comprises a shortened EF-1α promoter and one or more introns.

29. The method of any one of claims 20-28, wherein the WPRE sequence is modified.

30. The method of claim 29, wherein the WPRE sequence is WPRE mut6delATG.

31. The method of any one of claims 17-30, wherein the poly A is a bovine growth hormone (BGH) poly A.

32. The method of any one of the preceding claims, wherein the rAAV vector is administered by intravenous, subcutaneous, or transdermal administration.

33. The method of claim 32, wherein the rAAV is administered by intravenous administration.

34. The method of any one of the preceding claims, wherein following administration of the rAAV vector, the subject has detectable α-GAL in the serum for at least 5 weeks, 10 weeks, 15 weeks, 18 weeks, 20 weeks, 24 weeks, 26 weeks, 30 weeks, 40 weeks, 1 year, 5 years, 10 years or 15 years.

35. The method of claim 34, wherein the subject has detectable α-GAL in the serum for greater than 18 weeks.

36. The method of any one of the preceding claims, wherein the rAAV is administered to the subject at a dose ranging from about 1×10¹⁰ vg/kg (vector genomes/body weight) to about 1×10¹⁴ vg/kg (vector genomes/body weight).

37. The method of claim 36, wherein the rAAV is administered to the subject at a dose ranging from about 1.0×10¹⁰ vg/kg to 5.0×10¹³ vg/kg.

38. A method for alleviating or ameliorating a gastrointestinal symptom in a subject diagnosed with Fabry disease comprising administering to the subject a composition comprising a recombinant adeno-associated viral vector (rAAV) that comprises a polynucleotide encoding α-GAL enzyme or a variant thereof, wherein the subject diagnosed with Fabry disease has or is developing one or more gastrointestinal symptoms.

39. The method of claim 38, wherein the gastrointestinal symptoms include intestinal dysmotility, impaired autonomic function, vasculopathy and myopathy.

40. The method of claim 39, wherein administering the rAAV to the subject reverses vacuolization in the gastrointestinal tract.

41. The method of claim 9, wherein the intraepidermal nerve fiber density is preserved.

42. The method of claim 6, wherein the nerve fiber is dorsal nerve root and the vacuolization in the dorsal nerve root is reserved.

43. The method of any one of claims 38-40, wherein the rAAV vector is a rAAV vector packaged in a capsid with broad tissue tropism, the vector comprising:
a. a 5′ inverted terminal repeat (ITR);
b. a ubiquitous promoter;
c. the polynucleotide encoding α-GAL enzyme;
d. a poly A; and
e. a 3′ ITR.

44. The method of any one of claims 38-40, wherein the rAAV vector is a rAAV vector packaged in a capsid with broad tissue tropism, the vector comprising:
a. a 5′ inverted terminal repeat (ITR);
b. a liver specific promoter;
c. the polynucleotide encoding α-GAL enzyme;
d. a poly A; and
e. a 3′ ITR.

45. The method of any one of claims 38-40, wherein the rAAV vector is a rAAV vector packaged in a capsid with tropism specific to the nervous system, the vector comprising:
a. a 5′ inverted terminal repeat (ITR);
b. a ubiquitous promoter;
c. the polynucleotide encoding α-GAL enzyme;
d. a poly A; and
e. a 3′ ITR.

46. The method of any one of claims 43-45, wherein the vector further comprises a woodchuck hepatitis virus posttranscriptional regulatory element (WPRE).

47. The method of any one of claims 43-45, wherein the polynucleotide encodes α-GAL enzyme comprising an amino acid sequence selected from SEQ ID Nos: 2, 3, 6 and 7

48. The method of any one of claims 43-47, wherein the polynucleotide encoding α-GAL enzyme comprises a nucleotide sequence of SEQ ID Nos: 9, 10, 12 and 13.

49. The method of any one of claims 43-48, wherein the AAV capsid is a wide-tropism AAV capsid selected from an AAV1 capsid, AAV2 capsid, AAV3 capsid, AAV4 capsid, AAV5 capsid, AAV6 capsid, AAV7 capsid, AAV8 capsid, or AAV9 capsid.

50. The method of claim 49, wherein the wide-tropism AAV capsid is AAV9 capsid.

51. The method of claim 50, wherein the AAV9 capsid is naturally occurring or engineered.

52. The method of any one of claims 43-51, wherein the ubiquitous promoter comprises a chicken β actin (CBA) promoter, a EF-1α promoter, a PGK promoter, a UBC promoter, a LSE beta-glucuronidase (GUSB) promoter, a ubiquitous chromatin opening element (UCOE) promoter, a cyto-megalo-virus (CMV) enhancer, a chicken beta actin promoter, or one or more introns.

53. The method of claim 52, wherein the ubiquitous promoter comprising a cyto-megalo-virus (CMV) enhancer, chicken beta actin promoter, and a rabbit beta globin intron.

54. The method of claim 52, wherein the ubiquitous promoter comprises a shortened EF-1α promoter and one or more introns.

55. The method of any one of claims 46-54, wherein the WPRE sequence is modified.

56. The method of claim 55, wherein the WPRE sequence is WPRE mut6delATG.

57. The method of any one of claims 43-56, wherein the poly A is a bovine growth hormone (BGH) poly A.

58. The method of any one of claims 43-57, wherein the rAAV vector is administered by intravenous, subcutaneous, or transdermal administration.

59. The method of claim 58, wherein the rAAV is administered by intravenous administration.

60. The method of any one of claims 43-59, wherein the rAAV is administered to the subject at a dose ranging from about 1×10¹⁰ vg/kg (vector genomes/body weight) to about 1×10¹⁴ vg/kg (vector genomes/body weight).

61. The method of claim 60, wherein the rAAV is administered to the subject at a dose ranging from about 1.0×10¹⁰ vg/kg to 5.0×10¹³ vg/kg.
